(19) United States
(12) Patent Application Publication
(10) Pub. No.: US 2010/0029609 A1
(43) Pub. Date: Feb. 4, 2010

(54) BIARYL SULFONAMIDE DERIVATIVES

(76) Inventors: Frederic Berst, Altkirch (FR); Philipp Grosche, Inzlingen (DE); Philipp Janser, Basel (CH); Frederic Zecri, Bartenheim (FR); Birgit Bollbuck, Weil am Rhein (DE)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/440,143

(22) PCT Filed: Sep. 6, 2007
(86) PCT No.: PCT/EP07/59321

§ 371 Date: Mar. 5, 2009

(30) Foreign Application Priority Data
Sep. 8, 2006 (EP) 06120403.8

(51) Int. Cl. A61K 31/397; C07D 207/00; A61K 31/40; C07D 265/30; A61K 31/535; C07D 205/04; A61P 25/00

(52) U.S. Cl. 514/210.17; 548/532; 514/423; 544/159; 514/237.5; 548/953

(57) ABSTRACT

A compound of formula (I) or a pharmaceutically acceptable salt or prodrug ester thereof:

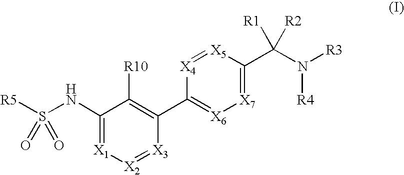

wherein the groups R1-R5, R10 and X₁-X₇ are as defined in the specification.

The present invention relates to biaryl sulfonamide compounds, to processes for their production, to their use as pharmaceuticals and to pharmaceutical compositions comprising them.

More particularly the present invention provides in a first aspect a compound of formula I or a pharmaceutically acceptable salt or a pharmaceutically-acceptable and -cleavable ester, or acid or amine addition salt thereof:

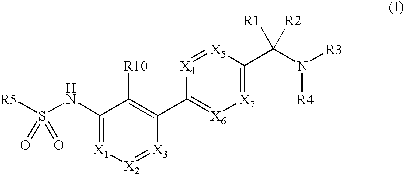

wherein

X₁, X₂, X₃, X₄, X₅, X₆ and X₇ are each independently selected from N or CR6, R6 in each case being independently selected from H, halo, cyano, OH or optionally substituted (C₁-C₆ alkyl, C₁-C₆ alkoxy, aryl C₁-C₆ alkoxy, heteroaryl C₁-C₆ alkoxy, C₁-C₆ alkylamine),

the optional substituents on R6 being selected from C₁-C₆ alkoxy, OH, halo, cyano, sulfonyl, C₁-C₆ alkyl, amino, mercapto, COOH;

R1 and R2 are each independently selected from H or C₁-C₆ alkyl, or taken together are O;

R3 is C₁-C₆ alkyl optionally substituted in any position by one or more substituents R3′,

R3′ being independently selected from COOR11, CON(R12)₂, hydroxyl, amino, aryl, heteroaryl, cycloalkyl, heterocycloalkyl, aryl C₁-C₆alkyl, heteroaryl C₁-C₆alkyl, C₁-C₆alkyl, C₁-C₆ alkoxy, halo, cyano, mercapto, and sulfonyl,

the optional substituents R3′ themselves being optionally substituted once or more by COOR11, CON(R12)₂, hydroxyl, amino, aryl, heteroaryl, cycloalkyl, heterocycloalkyl, aryl C₁-C₆ alkyl, heteroaryl C₁-C₆alkyl, C₁-C₆ alkyl, C₁-C₆ alkoxy, halo, cyano, mercapto, sulfonyl;

two R3′ may form together with the carbon atoms to which they are attached a 3-8 membered saturated or unsaturated carbocyclic ring optionally containing up to 2 ring members selected from CO, CHCOOR11, NR12, O, S, SO or SO₂;

wherein R11 is independently H, C₁-C₆ alkyl or benzyl; and R12 is independently H, OH, C₁-C₆ alkyl, benzyl, or acyl;

R4 is H, acyl or C₁-C₆ alkyl;

or R3 and R4 are linked together to form a 4, 5, 6 or 7 membered carbocyclic or heterocyclic ring which is optionally substituted by one or more groups R3′;

R5 is optionally substituted aryl or heteroaryl,

the optional substituents on R5 being one or more groups independently selected from halo, C₁-C₆ alkyl, NO₂, C₁-C₆ alkoxy, cyano, amino, sulfonyl, aryl, heteroaryl, mercapto, wherein the substituents on R5 are themselves optionally substituted by halo, NO₂, C₁-C₆ alkoxy, cyano, amino, sulfonyl, aryl or heteroaryl;

R10 is H or optionally substituted (C₁-C₆ alkyl, C₁-C₆ alkoxy, aryl C₁-C₆ alkoxy, heteroaryl C₁-C₆ alkoxy, C₁-C₆ alkylamine),

the optional substituents on R10 being selected from C₁-C₆ alkoxy, OH, halo, cyano, sulfonyl, C₁-C₆ alkyl, amino, mercapto, COOH.

The following significances are preferred independently, collectively or in any combination or sub-combination:
(i) X₁-X₇ are all CR6;
(ii) X₁-X₇ are all selected from CH, CCH₃ or COCH₃;
(iii) R1 and R2 taken together are O;
(iv) R1 and R2 are both H;
(v) R4 is H or methyl;
(vi) R4 is H;
(vii) R5 is optionally substituted aryl;
(viii) R5 is selected from optionally substituted phenyl, naphthyl, benzofuranyl, benzothienyl, thienyl, thiazolyl, pyrazolyl, imidazolyl;
(ix) R5 is optionally substituted phenyl;
(x) R5 is optionally substituted naphthyl;
(xi) R5 is phenyl having at least 2 substituents, at least one of which is halo and at least one of which is methyl;
(xiia) R6 is H, C₁-C₆ alkyl, C₁-C₆ alkoxy, CF₃, halo, OH;
(xiib) R6 is H, C₁-C₆ alkyl or C₁-C₆ alkoxy;
(xiic) R6 is H or C₁-C₆ alkyl;
(xiid) R6 is H or methyl;
(xiie) R6 is methyl and H in a ratio of 1:6, or 2:5;
(xiii) R10 is H or optionally substituted C₁-C₆ alkyl;
(xiv) R10 is H;
(xiva) R12 is independently H, or OH;
(xivb) R12 is independently H, or acyl;
(xivc) R12 is independently H, C₁-C₆ alkyl, or benzyl;
(xivd) R12 is independently H, C₁-C₆ alkyl, benzyl or acyl;

In a preferred embodiment the invention provides a compound of formula II or a pharmaceutically acceptable salt or a pharmaceutically-acceptable and -cleavable ester, or acid or amine addition salt thereof:

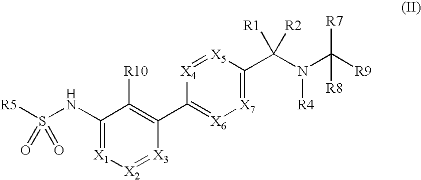

wherein X₁-X₇, R1, R2, R4, R5 and R10 are as defined with respect to formula I;

R7 is selected from H or optionally substituted C₁-C₆ alkyl, aryl, aryl C₁-C₆ alkyl, heteroaryl, heteroaryl C₁-C₆ alkyl,

the optional substituents on R7 being selected from OH, C₁-C₆ alkoxy and N(R12)₂; R12 being independently as defined above;

R8 is selected from H or C₁-C₆ alkyl;

or R7 and R8 form together with the carbon atoms to which they are attached a 3-8 membered saturated or unsaturated ring optionally containing up to 2 ring members selected from CO, CHCOOH, CHCOOR11, NR12, O, S, SO or SO₂;

R9 is COOR11, CON(R12)₂ or tetrazole.

In addition to the significances (i) to (xiv) defined above, the following significances are preferred independently, collectively or in any combination or sub-combination:

(xv) R7 is CH₂OH, (CH₂)₁₋₄N(R12)₂, (CH₂)₁₋₂N(R12)₂, isopropyl, ethyl, phenyl, benzyl or methyl;

(xvi) R7 is CH₂OH or CH₂N(R12)₂;

(xvii) R8 is H or methyl;

(xviii) R8 is H;

(xix) R9 is COOR11;

(xx) R11 is H, methyl or ethyl;

(xxia) R12 is H, methyl,ethyl, propyl, butyl or acetyl;

(xxib) R12 is H, methyl, C₁₋₆alkyl-CO or C₁₋₄alkoxy-CO;

(xxic) R12 is H, methyl, C₁₋₄alkyl-CO or acetyl (CH₃CO);

(xxid) R12 is H, benzyloxycarbonyl or t-butoxycarbonyl.

In a preferred embodiment the invention provides a compound of formula III or a pharmaceutically acceptable salt or a pharmaceutically-acceptable and -cleavable ester, or acid or amine addition salt thereof:

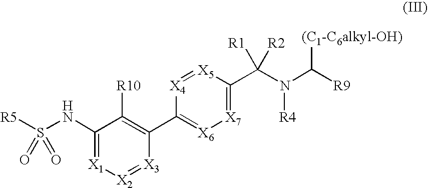

wherein X₁-X₇, R1, R2, R4, R5, R9 and R10 are as defined with respect to formula (I).

The above defined significances (i)-(xxi) apply also to the compounds of formula (III), (IIIa). (IIIb), and (IIIc).

In another preferred embodiment the invention provides a compound of formula (IIIa) or a pharmaceutically acceptable salt or a pharmaceutically-acceptable and -cleavable ester, or acid or amine addition salt thereof;

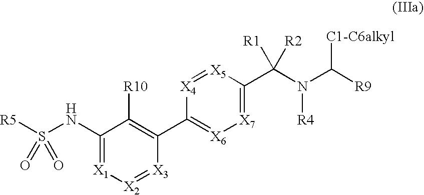

wherein X₁-X₇, R1, R2, R4, R5, R9 and R10 are as defined above.

In another preferred embodiment the invention provides a compound of formula (IIIb) or a pharmaceutically acceptable salt or a pharmaceutically-acceptable and -cleavable ester, or acid or amine addition salt thereof;

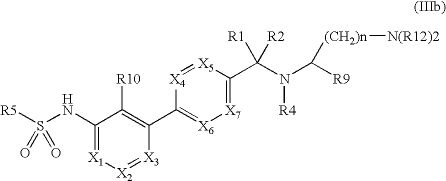

wherein X₁-X₇, R1, R2, R4, R5, R9, R10 and R12 are as defined above, and

wherein n is 1, 2, 3 or 4, preferably 1, 2 or 4, more preferably 1 or 2.

In another preferred embodiment the invention provides a compound of formula (IIIc) or a pharmaceutically acceptable salt or a pharmaceutically-acceptable and -cleavable ester, or acid or amine addition salt thereof;

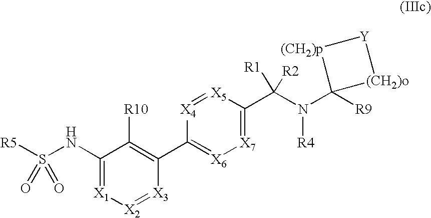

wherein X₁-X₇, R1, R2, R4, R5, R9 and R10 are as defined above,

o and p is an integer and is independently selected from 0, 1, 2, 3, 4 or 5 with the proviso that the sum of o+p is from 1 to 5, more preferably o+p is from 1 to 4; and Y is CH₂, CO, CHCOOH, CHCOOR11, NR12, O, S, SO or SO₂.

The compounds of the invention may exist in free form or in salt form, e.g. addition salts with e.g. organic or inorganic acids, for example, hydrochloric acid or acetic acid, or salts obtainable when R3 comprises COOH, with a base, e.g. alkali salts such as sodium or potassium, or unsubstituted or substituted ammonium salts, e.g. N-methyl-D-glucamine or D-glucamine.

It will be appreciated that the compounds of the invention may exist in the form of optical isomers, racemates or diastereoisomers. It is to be understood that the present invention embraces all enantiomers and conformers and their mixtures. Similar considerations apply in relation to starting materials exhibiting asymmetric carbon atoms as mentioned above.

By a pharmaceutically-acceptable and -cleavable ester or a physiologically hydrolysable derivative of a compound of formula I is meant a compound which is hydrolysable under physiological conditions to yield a compound of formula I and a by-product which is itself physiologically acceptable, e.g. an ester which is hydrolyzed to yield a compound of formula I and a non-toxic alcohol at the desired dosage levels.

For the avoidance of doubt, the terms listed below are to be understood to have the following meaning throughout the present description and claims:

The term “lower”, when referring to organic radicals or compounds means a compound or radical which may be branched or unbranched with up to and including 7 carbon atoms.

An alkyl may be branched, unbranched or cyclic. C₁-C₆ alkyl represents, for example: methyl, ethyl, propyl, butyl, isopropyl, isobutyl, tertiary butyl or 2,2-dimethylpropyl. In accordance to the foregoing, a cycloalkyl represents a cyclic hydrocarbon containing from 3 to 12 ring atoms preferably from 3 to 6 ring atoms. Cycloalkyl represents, for example: cyclopropyl, cyclobutyl, cyclopentyl, or cyclohexyl. The cycloalkyl may optionally be substituted.

An alkoxy group may be branched or unbranched. C₁-C₆ alkoxy represents, for example: methoxy, ethoxy, propoxy, butoxy, isopropoxy, isobutoxy or tertiary butoxy. Alkoxy includes cycloalkyloxy and cycloalkyl-alkyloxy.

An alkene, alkenyl or alkenoxy group is branched or unbranched and contains 2 to 7 carbon atoms, preferably 2 to 4 carbon atoms and contains at least one carbon-carbon double bond. Alkene, alkenyl or alkenoxy represents for example vinyl, prop-1-enyl, allyl, butenyl, isopropenyl or isobutenyl and the oxy equivalents thereof.

An alkyne or alkynyl group is branched or unbranched and contains 2 to 7 carbon atoms, preferably 1 to 4 carbon atoms and contains at least one carbon-carbon triple bond. Lower alkyne or lower alkynyl or lower alkenyloxy represents for example ethynyl or propynyl.

In the present application, oxygen containing substituents, e.g. alkoxy, alkenyloxy, alkynyloxy, carbonyl, etc. encompass their sulphur containing homologues, e.g. thioalkyl, alkyl-thioalkyl, thioalkenyl, alkenyl-thioalkyl, thioalkynyl, thiocarbonyl, sulphone, sulphoxide etc.

Halo or halogen represents chloro, fluoro, bromo or iodo. Preferably halo or halogen represents chloro or fluoro.

As used herein acyl is a radical R_{d}CO wherein R_{d} is H, C₁₋₆alkyl, C₃₋₆cycloalkyl, C₃₋₆cycloalkyloxy, C₁₋₆alkoxy, phenyl, phenyloxy, benzyl or benzyloxy, preferably acyl is C₁₋₆alkyl-CO, C₁₋₆alkoxy-CO, benzyloxy-CO or benzyl-CO, more preferably C₁₋₆alkyl-CO or C₁₋₄alkoxy-CO, particularly C₁₋₄alkyl-CO, C₁₋₄alkoxy-CO, t-butoxycarbonyl or acetyl (CH₃CO).

Aryl represents carbocyclic aryl or biaryl.

Carbocyclic aryl is an aromatic cyclic hydrocarbon containing from 6 to 18 ring atoms. It can be monocyclic, bicyclic or tricyclic, for example naphthyl, phenyl, or phenyl mono-, di- or trisubstituted by one, two or three substituents.

Heterocyclic aryl or heteroaryl is an aromatic monocyclic or bicyclic hydrocarbon containing from 5 to 18 ring atoms one or more of which are heteroatoms selected from O, N or S. Preferably there are one to three heteroatoms. Heterocyclic aryl represents, for example: pyridyl, indolyl, quinoxalinyl, quinolinyl, isoquinolinyl, benzothienyl, benzofuranyl, benzthiophenyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, oxadiazolyl, benzimidazolyl, benzthiazolyl, benzoxazolyl, Heterocyclic aryl also includes such substituted radicals.

Heterocycloalkyl represents a mono-, di- or tricyclic hydrocarbon which may be saturated or unsaturated and which contains one or more, preferably one to three heteroatoms selected from O, N or S. Preferably it contains between three and 18 ring atoms, more preferably between 3 and 8 ring atoms. Heterocycloalkyl represents for example morpholinyl, piperazinyl, piperidinyl, imidazolidinyl, pyrrolidinyl, pyrazolidinyl. The term heterocycloalkyl is intended also to include bridged heterocycloalkyl groups such as 8-aza-bicyclo[3.2.1]oct-8-yl or 2,6-diaza-tricyclo[3.3.1.1*3,7*]dec-1-yl.

Pharmaceutically acceptable salts include acid addition salts with conventional acids, for example mineral acids, e.g. hydrochloric acid, sulfuric or phosphoric acid, or organic acids, for example aliphatic or aromatic carboxylic or sulfonic acids, e.g. acetic, trifluoroacetic, propionic, succinic, glycolic, lactic, malic, tartaric, citric, ascorbic, maleic, fumaric, hydroxylmaleic, pyruvic, pamoic, methanesulfonic, toluenesulfonic, naphthalenesulfonic, sulfanilic or cyclohexylsulfamic acid; also amino acids, such as arginine and lysine. For compounds of the invention having acidic groups, for example a free carboxy group, pharmaceutically acceptable salts also represent metal or ammonium salts, such as alkali metal or alkaline earth metal salts, e.g. sodium, potassium, magnesium or calcium salts, as well as ammonium salts, which are formed with ammonia or suitable organic amines, e.g. N-methyl-D-glucamine or D-glucamine.

The agents of the invention which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding agents of the invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

Preferred compounds of formula (I) are:

(S)-3-Methyl-2-{[3′-(2,4,5-trichloro-benzenesulfonylamino)-biphenyl-4-carbonyl]-amino}-butyric acid,

(S)-2-{[3′-(3,4-Dichloro-benzenesulfonylamino)-biphenyl-4-carbonyl]-amino}-3-methyl-butyric acid,

(S)-3-Methyl-2-{[3′-(naphthalene-2-sulfonylamino)-biphenyl-4-carbonyl]-amino}-butyric acid,

{[3′-(4-Chloro-benzenesulfonylamino)-biphenyl-4-carbonyl]-amino}-acetic acid,

(S)-2-{[3′-(5-Chloro-naphthalene-2-sulfonylamino)-biphenyl-4-carbonyl]-amino}-3-methyl-butyric acid,

(S)-2-{[3′-(4-Chloro-3-methyl-benzenesulfonylamino)-biphenyl-4-carbonyl]-amino}-3-methyl-butyric acid,

(S)-2-{[3′-(2,4-Dimethyl-benzenesulfonylamino)-biphenyl-4-carbonyl]-amino}-3-methyl-butyric acid,

(S)-2-{[3′-(2,4-Dichloro-5-methyl-benzenesulfonylamino)-biphenyl-4-carbonyl]-amino}-3-methyl-butyric acid,

(S)-2-{[3′-(2,5-Dichloro-3,6-dimethyl-benzenesulfonylamino)-biphenyl-4-carbonyl]-amino}-3-methyl-butyric acid,

(S)-2-{[3′-(4-Chloro-3-trifluoromethyl-benzenesulfonylamino)-biphenyl-4-carbonyl]-amino}-3-methyl-butyric acid,

(S)-3-Methyl-2-{[3′-(2,4,6-trimethyl-benzenesulfonylamino)-biphenyl-4-carbonyl]-amino}-butyric acid,

(S)-2-{[3′-(2, 3-Dichloro-benzenesulfonylamino)-biphenyl-4-carbonyl]-amino}-3-methyl-butyric acid,

(S)-2-{[3′-(3-Chloro-2-methyl-benzenesulfonylamino)-biphenyl-4-carbonyl]-amino}-3-methyl-butyric acid,

(S)-3-Methyl-2-{[3′-(2-methyl-5-nitro-benzenesulfonylamino)-biphenyl-4-carbonyl]-amino}-butyric acid,

(S)-2-{[3′-(4-Methoxy-2,3,6-trimethyl-benzenesulfonylamino)-biphenyl-4-carbonyl]-amino}-3-methyl-butyric acid,

(S)-2-{[3′-(3,5-Dichloro-benzenesulfonylamino)-biphenyl-4-carbonyl]-amino}-3-methyl-butyric acid,

(S)-2-{[3′-(2,4-Dichloro-benzenesulfonylamino)-biphenyl-4-carbonyl]-amino}-3-methyl-butyric acid,

(S)-3-Methyl-2-[(3′-pentamethylbenzenesulfonylamino-biphenyl-4-carbonyl)-amino]-butyric acid,

(S)-3-Methyl-2-{[3′-(2,3,5,6-tetramethyl-benzenesulfonylamino)-biphenyl-4-carbonyl]-amino}-butyric acid,

(S)-2-{[3′-(2,5-Dimethyl-benzenesulfonylamino)-biphenyl-4-carbonyl]-amino}-3-methyl-butyric acid,

(S)-2-{[3′-(4-Chloro-2,5-dimethyl-benzenesulfonylamino)-biphenyl-4-carbonyl]-amino}-3-methyl-pentanoic acid,

(S)-2-{[3′-(4-Chloro-2,5-dimethyl-benzenesulfonylamino)-biphenyl-4-carbonyl]-amino}-3-methyl-butyric acid,

{[3′-(4-Chloro-2,5-dimethyl-benzenesulfonylamino)-biphenyl-4-carbonyl]-amino}-acetic acid,

{[3′-(4-Chloro-2,5-dimethyl-benzenesulfonylamino)-biphenyl-4-carbonyl]-amino}-acetatemethyl-((2S,3R,4R,5R)-2,3,4,5,6-pentahydroxy-hexyl)-ammonium,

(R)-2-{[3′-(4-Chloro-2,5-dimethyl-benzenesulfonylamino)-biphenyl-4-carbonyl]-amino}-3-methyl-butyric acid,

(S)-2-{[3′-(4-Chloro-2,5-dimethyl-benzenesulfonylamino)-biphenyl-4-carbonyl]-amino}-propionic acid,

(S)-2-{[3′-(4-Chloro-2,5-dimethyl-benzenesulfonylamino)-biphenyl-4-carbonyl]-amino}-3-phenyl-propionic acid,

(S)-1-[3′-(4-Chloro-2,5-dimethyl-benzenesulfonylamino)-biphenyl-4-carbonyl]-pyrrolidine-2-carboxylic acid,

(S)-2-{[3′-(4-Chloro-2,5-dimethyl-benzenesulfonylamino)-biphenyl-4-carbonyl]-amino}-3-hydroxy-propionic acid,

(S)-2-{[3′-(4-Chloro-2,5-dimethyl-benzenesulfonylamino)-biphenyl-4-carbonyl]-amino}-3-hydroxy-propionate methyl-((2S,3R,4R,5R)-2,3,4,5,6-pentahydroxy-hexyl)-ammonium,

{[3′-(4-Chloro-2,5-dimethyl-benzenesulfonylamino)-biphenyl-4-carbonyl]-methyl-amino}-acetic acid,

3-{[3′-(4-Chloro-2,5-dimethyl-benzenesulfonylamino)-biphenyl-4-carbonyl]-amino}-propionic acid,

(S)-3-{[3′-(4-Chloro-2,5-dimethyl-benzenesulfonylamino)-biphenyl-4-carbonyl]-amino}-butyric acid,

(S)-2-{[3′-(4-Chloro-2,5-dimethyl-benzenesulfonylamino)-biphenyl-4-carbonyl]-amino}-butyric acid,

(R)-3-{[3′-(4-Chloro-2,5-dimethyl-benzenesulfonylamino)-biphenyl-4-carbonyl]-amino}-4-methyl-pentanoic acid,

2-{[3′-(4-Chloro-2,5-dimethyl-benzenesulfonylamino)-biphenyl-4-carbonyl]-amino}-2-methyl-propionic acid,

(S)-3-{[3′-(4-Chloro-2,5-dimethyl-benzenesulfonylamino)-biphenyl-4-carbonyl]-amino}-4-phenyl-butyric acid,

(R)-3-{[3′-(4-Chloro-2,5-dimethyl-benzenesulfonylamino)-biphenyl-4-carbonyl]-amino}-3-phenyl-propionic acid,

3′-(4-Chloro-2,5-dimethyl-benzenesulfonylamino)-biphenyl-4-carboxylic acid (3-methoxy-propyl)-amide,

3′-(4-Chloro-2,5-dimethyl-benzenesulfonylamino)-biphenyl-4-carboxylic acid ((S)-1-carbamoyl-2-methyl-propyl)-amide,

3′-(4-Chloro-2,5-dimethyl-benzenesulfonylamino)-biphenyl-4-carboxylic acid ((S)-2-methyl-1-methylcarbamoyl-propyl)-amide,

3′-(4-Chloro-2,5-dimethyl-benzenesulfonylamino)-biphenyl-4-carboxylic acid ((S)-1-dimethylcarbamoyl-2-methyl-propyl)-amide,

{[3′-(4-Chloro-2,5-dimethyl-benzenesulfonylamino)-2′-methyl-biphenyl-4-carbonyl]-amino}-acetic acid,

(S)-2-{[3′-(4-Chloro-2,5-dimethyl-benzenesulfonylamino)-2′-methyl-biphenyl-4-carbonyl]-amino}-3-hydroxy-propionic acid,

{[3′-(4-Chloro-2,5-dimethyl-benzenesulfonylamino)-2-methyl-biphenyl-4-carbonyl]-amino}-acetic acid,

(S)-2-{[3′-(4-Chloro-2,5-dimethyl-benzenesulfonylamino)-2-methyl-biphenyl-4-carbonyl]-amino}-3-hydroxy-propionic acid,

(S)-2-{[3′-(4-Chloro-2,5-dimethyl-benzenesulfonylamino)-3-methyl-biphenyl-4-carbonyl]-amino}-3-hydroxy-propionic acid,

(R)-2-{[3′-(4-Chloro-2,5-dimethyl-benzenesulfonylamino)-3-methyl-biphenyl-4-carbonyl]-amino}-3-hydroxy-propionic acid,

(2S,3R)-2-{[3′-(4-Chloro-2,5-dimethyl-benzenesulfonylamino)-3-methyl-biphenyl-4-carbonyl]-amino}-3-hydroxy-butyric acid,

(S)-3-tert-Butoxy-2-{[3′-(4-chloro-2,5-dimethyl-benzenesulfonylamino)-3-methyl-biphenyl-4-carbonyl]-amino}-propionic acid,

3-{[3′-(4-Chloro-2,5-dimethyl-benzenesulfonylamino)-3-methyl-biphenyl-4-carbonyl]-amino}-azetidine-1,3-dicarboxylic acid 1-tert-butyl ester 3-ethyl ester,

3-{[3′-(4-Chloro-2,5-dimethyl-benzenesulfonylamino)-3-methyl-biphenyl-4-carbonyl]-amino}-azetidine-1,3-dicarboxylic acid mono-tert-butyl ester,

3-{[3′-(4-Chloro-2,5-dimethyl-benzenesulfonylamino)-3-methyl-biphenyl-4-carbonyl]-amino}-azetidine-3-carboxylic acid,

3-{[3′-(4-Chloro-2,5-dimethyl-benzenesulfonylamino)-3-methyl-biphenyl-4-carbonyl]-amino}-azetidine-3-carboxylic acid methyl ester,

3-{[3′-(4-Chloro-2,5-dimethyl-benzenesulfonylamino)-3-methyl-biphenyl-4-carbonyl]-amino}-azetidine-3-carboxylic acid ethyl ester,

3-{[3′-(4-Chloro-2,5-dimethyl-benzenesulfonylamino)-3-methyl-biphenyl-4-carbonyl]-amino}-1-methyl-azetidine-3-carboxylic acid ethyl ester,

3-{[3′-(4-Chloro-2,5-dimethyl-benzenesulfonylamino)-3-methyl-biphenyl-4-carbonyl]-amino}-1-methyl-azetidine-3-carboxylic acid,

1-Acetyl-3-{[3′-(4-chloro-2,5-dimethyl-benzenesulfonylamino)-3-methyl-biphenyl-4-carbonyl]-amino}-azetidine-3-carboxylic acid,

1-{[3′-(4-Chloro-2,5-dimethyl-benzenesulfonylamino)-3-methyl-biphenyl-4-carbonyl]-amino}-cyclopropanecarboxylic acid,

1-[3′-(4-Chloro-2,5-dimethyl-benzenesulfonylamino)-3-methyl-biphenyl-4-carbonyl]-azetidine-3-carboxylic acid,

(2S,3S)-2-{[3′-(4-Chloro-2,5-dimethyl-benzenesulfonylamino)-3-methyl-biphenyl-4-carbonyl]-amino}-3-hydroxy-butyric acid,

(S)-2-{[3′-(4-Chloro-2,5-dimethyl-benzenesulfonylamino)-3-methyl-biphenyl-4-carbonyl]-amino}-3-methoxy-propionic acid,

(S)-2-{[3′-(4-Chloro-2,5-dimethyl-benzenesulfonylamino)-3-methyl-biphenyl-4-carbonyl]-amino}-3-hydroxy-3-methyl-butyric acid,

(S)-2-{[3′-(4-Chloro-2,5-dimethyl-benzenesulfonylamino)-3-methyl-biphenyl-4-carbonyl]-amino}-butyric acid,

(S)-2-{[3′-(4-Chloro-2,5-dimethyl-benzenesulfonylamino)-3-methyl-biphenyl-4-carbonyl]-amino}-propionic acid,

{[3′-(4-Chloro-2,5-dimethyl-benzenesulfonylamino)-3-methyl-biphenyl-4-carbonyl]-amino}-acetic acid,

3′-(4-Chloro-2,5-dimethyl-benzenesulfonylamino)-3-methyl-biphenyl-4-carboxylic acid cyanomethyl-amide,

3′-(4-Chloro-2,5-dimethyl-benzenesulfonylamino)-3-methyl-biphenyl-4-carboxylic acid (1H-tetrazol-5-ylmethyl)-amide,

3′-(4-Chloro-2,5-dimethyl-benzenesulfonylamino)-3-methyl-biphenyl-4-carboxylic acid (2-hydroxy-2-methyl-propyl)-amide,

{[5′-(4-Chloro-2,5-dimethyl-benzenesulfonylamino)-2′-methyl-biphenyl-4-carbonyl]-amino}-acetic acid,

(S)-2-{[5′-(4-Chloro-2,5-dimethyl-benzenesulfonylamino)-2′-methyl-biphenyl-4-carbonyl]-amino}-3-hydroxy-propionic acid,

{[3′-(4-Chloro-2,5-dimethyl-benzenesulfonylamino)-3-methoxy-biphenyl-4-carbonyl]-amino}-acetic acid,

(S)-2-{[3′-(4-Chloro-2,5-dimethyl-benzenesulfonylamino)-3-methoxy-biphenyl-4-carbonyl]-amino}-3-hydroxy-propionic acid,

(S)-2-({5-[3-(4-Chloro-2,5-dimethyl-benzenesulfonylamino)-phenyl]-pyrazine-2-carbonyl}-amino)-3-hydroxy-propionic acid,

(S)-2-{[3′-(4-Chloro-2,5-dimethyl-benzenesulfonylamino)-3-isobutoxy-biphenyl-4-carbonyl]-amino}-3-hydroxy-propionic acid,

(S)-2-{[3′-(4-Chloro-2,5-dimethyl-benzenesulfonylamino)-3-(2-methoxy-ethoxy)-biphenyl-4-carbonyl]-amino}-3-hydroxy-propionic acid,

(S)-2-{[3′-(4-Chloro-2,5-dimethyl-benzenesulfonylamino)-3-propoxy-biphenyl-4-carbonyl]-amino}-3-hydroxy-propionic acid,

(S)-2-{[3′-(4-Chloro-2,5-dimethyl-benzenesulfonylamino)-3-(pyridin-3-ylmethoxy)-biphenyl-4-carbonyl]-amino}-3-hydroxy-propionic acid,

{4-[5-(4-Chloro-2,5-dimethyl-benzenesulfonylamino)-pyridin-3-yl]-benzoylamino}-acetic acid,

(S)-2-{4-[5-(4-Chloro-2,5-dimethyl-benzenesulfonylamino)-pyridin-3-yl]-benzoylamino}-3-hydroxy-propionic acid,

(S)-2-({5-[3-(4-Chloro-2,5-dimethyl-benzenesulfonylamino)-phenyl]-pyrazine-2-carbonyl}-amino)-3-hydroxy-propionic acid,

3′-(4-Chloro-2,5-dimethyl-benzenesulfonylamino)-biphenyl-4-carboxylic acid (2-hydroxy-ethyl)-amide,

2-{[3′-(4-Chloro-2,5-dimethyl-benzenesulfonylamino)-biphenyl-4-carbonyl]-amino}-3-hydroxy-propionic acid methyl ester,

3′-(4-Chloro-2,5-dimethyl-benzenesulfonylamino)-biphenyl-4-carboxylic acid (2-hydroxy-1-hydroxymethyl-1-methyl-ethyl)-amide,

3′-(4-Chloro-2,5-dimethyl-benzenesulfonylamino)-biphenyl-4-carboxylic acid (2-hydroxy-1-hydroxymethyl-ethyl)-amide,

2-{[3′-(4-Chloro-2,5-dimethyl-benzenesulfonylamino)-biphenyl-4-carbonyl]-amino}-3-hydroxy-2-methyl-propionic acid,

(S)-2-{[3′-(4-Chloro-2,5-dimethyl-benzenesulfonylamino)-biphenyl-4-carbonyl]-methyl-amino}-3-hydroxy-propionic acid,

(R)-2-{[3′-(4-Chloro-2,5-dimethyl-benzenesulfonylamino)-biphenyl-4-carbonyl]-methyl-amino}-3-hydroxy-propionic acid,

3′-(4-Chloro-2,5-dimethyl-benzenesulfonylamino)-biphenyl-4-carboxylic acid cyanomethyl-amide,

3′-(4-Chloro-2,5-dimethyl-benzenesulfonylamino)-biphenyl-4-carboxylic acid (1H-tetrazol-5-ylmethyl)-amide,

3′-(4-Chloro-2,5-dimethyl-benzenesulfonylamino)-biphenyl-4-carboxylic acid (3,3,3-trifluoro-2-hydroxy-propyl)-amide,

3′-(4-Chloro-2,5-dimethyl-benzenesulfonylamino)-biphenyl-4-carboxylic acid (2-fluoro-ethyl)-amide,

3′-(4-Chloro-2,5-dimethyl-benzenesulfonylamino)-biphenyl-4-carboxylic acid (2,2-difluoro-ethyl)-amide,

3′-(4-Chloro-2,5-dimethyl-benzenesulfonylamino)-biphenyl-4-carboxylic acid (2,2,2-trifluoro-ethyl)-amide,

3′-(4-Chloro-2,5-dimethyl-benzenesulfonylamino)-biphenyl-4-carboxylic acid (2-hydroxy-2-methyl-propyl)-amide,

3′-(4-Chloro-2,5-dimethyl-benzenesulfonylamino)-biphenyl-4-carboxylic acid (2-methoxy-1-methyl-ethyl)-amide,

3′-(4-Chloro-2,5-dimethyl-benzenesulfonylamino)-biphenyl-4-carboxylic acid ((S)-2-methoxy-1-methyl-ethyl)-amide,

3′-(4-Chloro-2,5-dimethyl-benzenesulfonylamino)-biphenyl-4-carboxylic acid (2-methoxy-ethyl)-amide,

3′-(4-Chloro-2,5-dimethyl-benzenesulfonylamino)-biphenyl-4-carboxylic acid (2-amino-2-methyl-propyl)-amide,

4-[3′-(4-Chloro-2,5-dimethyl-benzenesulfonylamino)-biphenyl-4-carbonyl]-piperazine-2-carboxylic acid,

(S)-2-{[3′-(Benzofuran-2-sulfonylamino)-biphenyl-4-carbonyl]-amino}-3-hydroxy-propionic acid,

(S)-2-{[3′-(Benzo[b]thiophene-3-sulfonylamino)-biphenyl-4-carbonyl]-amino}-3-hydroxy-propionic acid,

(S)-3-Hydroxy-2-{[3′-(thiophene-2-sulfonylamino)-biphenyl-4-carbonyl]-amino}-propionic acid,

(S)-2-{[3′-(2,4-Dimethyl-thiazole-5-sulfonylamino)-biphenyl-4-carbonyl]-amino}-3-hydroxy-propionic acid,

(S)-2-{[3′-(5-Chloro-1,3-dimethyl-1H-pyrazole-4-sulfonylamino)-biphenyl-4-carbonyl]-amino}-3-hydroxy-propionic acid,

(S)-2-{[3′-(1,2-Dimethyl-1H-imidazole-4-sulfonylamino)-biphenyl-4-carbonyl]-amino}-3-hydroxy-propionic acid,

(S)-3-Hydroxy-2-{[3′-(1,3,5-trimethyl-1H-pyrazole-4-sulfonylamino)-biphenyl-4-carbonyl]-amino}-propionic acid,

(S)-2-{[3′-(4,5-Dichloro-thiophene-2-sulfonylamino)-biphenyl-4-carbonyl]-amino}-3-hydroxy-propionic acid,

(S)-3-Hydroxy-2-{[3′-(thiophene-3-sulfonylamino)-biphenyl-4-carbonyl]-amino}-propionic acid,

(R)-2-{[3′-(4-Chloro-2,5-dimethyl-benzenesulfonylamino)-3,5-dimethyl-biphenyl-4-carbonyl]-amino}-3-hydroxy-propionic acid methyl ester,

(S)-2-{[3′-(4-Chloro-2,5-dimethyl-benzenesulfonylamino)-3,5-dimethyl-biphenyl-4-carbonyl]-amino}-3-hydroxy-propionic acid ethyl ester,

(S)-2-{[3′-(4-Chloro-2,5-dimethyl-benzenesulfonylamino)-3,5-dimethyl-biphenyl-4-carbonyl]-amino}-propionic acid methyl ester,

(S)-2-{[3′-(4-Chloro-2,5-dimethyl-benzenesulfonylamino)-3,5-dimethyl-biphenyl-4-carbonyl]-amino}-propionic acid ethyl ester,

(S)-2-{[3′-(4-Chloro-2,5-dimethyl-benzenesulfonylamino)-3,5-dimethyl-biphenyl-4-carbonyl]-amino}-3-hydroxy-propionic acid methyl ester,

(R)-2-{[3′-(4-Chloro-2,5-dimethyl-benzenesulfonylamino)-3,5-dimethyl-biphenyl-4-carbonyl]-amino}-propionic acid methyl ester,

(S)-2-{[3′-(4-Chloro-2,5-dimethyl-benzenesulfonylamino)-3,5-dimethyl-biphenyl-4-carbonyl]-amino}-butyric acid tert-butyl ester,

(S)-2-{[3′-(4-Chloro-2,5-dimethyl-benzenesulfonylamino)-3,5-dimethyl-biphenyl-4-carbonyl]-amino}-3-methoxy-propionic acid methyl ester,

{[3′-(4-Chloro-2,5-dimethyl-benzenesulfonylamino)-3,5-dimethyl-biphenyl-4-carbonyl]-amino}-acetic acid ethyl ester,

(S)-2-{[3′-(4-Chloro-2,5-dimethyl-benzenesulfonylamino)-3,5-dimethyl-biphenyl-4-carbonyl]-methyl-amino}-3-hydroxy-propionic acid methyl ester,

(S)-2-{[3′-(4-Chloro-2,5-dimethyl-benzenesulfonylamino)-3,5-dimethyl-biphenyl-4-carbonyl]-methyl-amino}-propionic acid methyl ester,

2-{[3′-(4-Chloro-2,5-dimethyl-benzenesulfonylamino)-3,5-dimethyl-biphenyl-4-carbonyl]-amino}-2-methyl-propionic acid methyl ester,

(S)-3-tert-Butoxycarbonylamino-2-{[3′-(4-chloro-2,5-dimethyl-benzenesulfonylamino)-3,5-dimethyl-biphenyl-4-carbonyl]-amino}-propionic acid methyl ester,

(R)-3-tert-Butoxycarbonylamino-2-{[3′-(4-chloro-2,5-dimethyl-benzenesulfonylamino)-3,5-dimethyl-biphenyl-4-carbonyl]-amino}-propionic acid methyl ester,

(S)-3-Amino-2-{[3′-(4-chloro-2,5-dimethyl-benzenesulfonylamino)-3,5-dimethyl-biphenyl-4-carbonyl]-amino}-propionic acid methyl ester hydrochloride,

(R)-3-Amino-2-{[3′-(4-chloro-2,5-dimethyl-benzenesulfonylamino)-3,5-dimethyl-biphenyl-4-carbonyl]-amino}-propionic acid methyl ester hydrochloride,

3-{[3′-(4-Chloro-2,5-dimethyl-benzenesulfonylamino)-3,5-dimethyl-biphenyl-4-carbonyl]-amino}-azetidine-1,3-dicarboxylic acid 1-tert-butyl ester 3-ethyl ester,

4-{[3′-(4-Chloro-2,5-dimethyl-benzenesulfonylamino)-3,5-dimethyl-biphenyl-4-carbonyl]-amino}-1-methyl-piperidine-4-carboxylic acid methyl ester,

4-{[3′-(4-Chloro-2,5-dimethyl-benzenesulfonylamino)-3,5-dimethyl-biphenyl-4-carbonyl]-amino}-tetrahydro-pyran-4-carboxylic acid ethyl ester,

1-{[3′-(4-Chloro-2,5-dimethyl-benzenesulfonylamino)-3,5-dimethyl-biphenyl-4-carbonyl]-amino}-cyclobutanecarboxylic acid ethyl ester,

1-{[3′-(4-Chloro-2,5-dimethyl-benzenesulfonylamino)-3,5-dimethyl-biphenyl-4-carbonyl]-amino}-cyclopropanecarboxylic acid ethyl ester,

3-{[3′-(4-Chloro-2,5-dimethyl-benzenesulfonylamino)-3,5-dimethyl-biphenyl-4-carbonyl]-amino}-azetidine-3-carboxylic acid methyl ester,

3-{[3′-(4-Chloro-2,5-dimethyl-benzenesulfonylamino)-3,5-dimethyl-biphenyl-4-carbonyl]-amino}-1-methyl-azetidine-3-carboxylic acid methyl ester,

(S)-2-{[3′-(4-Chloro-2,5-dimethyl-benzenesulfonylamino)-3,5-dimethyl-biphenyl-4-carbonyl]-amino}-3-hydroxy-propionic acid,

(S)-2-{[3′-(4-Chloro-2,5-dimethyl-benzenesulfonylamino)-3,5-dimethyl-biphenyl-4-carbonyl]-amino}-propionic acid,

(R)-2-{[3′-(4-Chloro-2,5-dimethyl-benzenesulfonylamino)-3,5-dimethyl-biphenyl-4-carbonyl]-amino}-3-hydroxy-propionic acid,

(R)-2-{[3′-(4-Chloro-2,5-dimethyl-benzenesulfonylamino)-3,5-dimethyl-biphenyl-4-carbonyl]-amino}-propionic acid,

(S)-2-{[3′-(4-Chloro-2,5-dimethyl-benzenesulfonylamino)-3,5-dimethyl-biphenyl-4-carbonyl]-amino}-butyric acid,

(S)-2-{[3′-(4-Chloro-2,5-dimethyl-benzenesulfonylamino)-3,5-dimethyl-biphenyl-4-carbonyl]-amino}-3-methoxy-propionic acid,

{[3′-(4-Chloro-2,5-dimethyl-benzenesulfonylamino)-3,5-dimethyl-biphenyl-4-carbonyl]-amino}-acetic acid,

(S)-2-{[3′-(4-Chloro-2,5-dimethyl-benzenesulfonylamino)-3,5-dimethyl-biphenyl-4-carbonyl]-methyl-amino}-3-hydroxy-propionic acid,

(S)-2-{[3′-(4-Chloro-2,5-dimethyl-benzenesulfonylamino)-3,5-dimethyl-biphenyl-4-carbonyl]-methyl-amino}-propionic acid,

(S)-3-tert-Butoxycarbonylamino-2-{[3′-(4-chloro-2,5-dimethyl-benzenesulfonylamino)-3,5-dimethyl-biphenyl-4-carbonyl]-amino}-propionic acid,

(R)-3-tert-Butoxycarbonylamino-2-{[3′-(4-chloro-2,5-dimethyl-benzenesulfonylamino)-3,5-dimethyl-biphenyl-4-carbonyl]-amino}-propionic acid,

(S)-3-Amino-2-{[3′-(4-chloro-2,5-dimethyl-benzenesulfonylamino)-3,5-dimethyl-biphenyl-4-carbonyl]-amino}-propionic acid,

(R)-3-Amino-2-{[3′-(4-chloro-2,5-dimethyl-benzenesulfonylamino)-3,5-dimethyl-biphenyl-4-carbonyl]-amino}-propionic acid,

3-{[3′-(4-Chloro-2,5-dimethyl-benzenesulfonylamino)-3,5-dimethyl-biphenyl-4-carbonyl]-amino}-azetidine-1,3-dicarboxylic acid mono-tert-butyl ester,

3-{[3′-(4-Chloro-2,5-dimethyl-benzenesulfonylamino)-3,5-dimethyl-biphenyl-4-carbonyl]-amino}-azetidine-3-carboxylic acid,

4-{[3′-(4-Chloro-2,5-dimethyl-benzenesulfonylamino)-3,5-dimethyl-biphenyl-4-carbonyl]-amino}-tetrahydro-pyran-4-carboxylic acid,

1-{[3′-(4-Chloro-2,5-dimethyl-benzenesulfonylamino)-3,5-dimethyl-biphenyl-4-carbonyl]-amino}-cyclobutanecarboxylic acid,

2-{[3′-(4-Chloro-2,5-dimethyl-benzenesulfonylamino)-3,5-dimethyl-biphenyl-4-carbonyl]-amino}-2-methyl-propionic acid,

1-{[3′-(4-Chloro-2,5-dimethyl-benzenesulfonylamino)-3,5-dimethyl-biphenyl-4-carbonyl]-amino}-cyclopropanecarboxylic acid,

3-{[3′-(4-Chloro-2,5-dimethyl-benzenesulfonylamino)-3,5-dimethyl-biphenyl-4-carbonyl]-amino}-1-methyl-azetidine-3-carboxylic acid,

3′-(4-Chloro-2,5-dimethyl-benzenesulfonylamino)-3,5-dimethyl-biphenyl-4-carboxylic acid ((S)-1-carbamoyl-ethyl)-amide,

3′-(4-Chloro-2,5-dimethyl-benzenesulfonylamino)-3,5-dimethyl-biphenyl-4-carboxylic acid ((S)-1-methylcarbamoyl-ethyl)-amide,

3′-(4-Chloro-2,5-dimethyl-benzenesulfonylamino)-3,5-dimethyl-biphenyl-4-carboxylic acid ((S)-1-carbamoyl-2-hydroxy-ethyl)-amide,

(S)-2-{[3′-(4-Chloro-2,5-dimethyl-benzenesulfonylamino)-3-ethyl-biphenyl-4-carbonyl]-amino}-propionic acid,

4-{[3′-(4-Chloro-2,5-dimethyl-benzenesulfonylamino)-3,5-dimethyl-biphenyl-4-carbonyl]-amino}-1-methyl-piperidine-4-carboxylic acid,

(S)-2-{[3′-(4-Chloro-2,5-dimethyl-benzenesulfonylamino)-biphenyl-4-ylmethyl]-amino}-3-hydroxy-propionic acid,

(R)-2-{[3′-(4-Chloro-2,5-dimethyl-benzenesulfonylamino)-biphenyl-4-ylmethyl]-amino}-3-hydroxy-propionic acid,

(S)-2-{[3′-(4-Chloro-2,5-dimethyl-benzenesulfonylamino)-3-methyl-biphenyl-4-ylmethyl]-amino}-3-hydroxy-propionic acid,

(R)-2-{[3′-(4-Chloro-2,5-dimethyl-benzenesulfonylamino)-3-methyl-biphenyl-4-ylmethyl]-amino}-3-hydroxy-propionic acid,

(S)-2-{1-[3′-(4-Chloro-2,5-dimethyl-benzenesulfonylamino)-biphenyl-4-yl]-ethylamino}-3-hydroxy-propionic acid,

(S)-2-{1-[3′-(4-Chloro-2,5-dimethyl-benzenesulfonylamino)-biphenyl-4-yl]-pentylamino}-3-hydroxy-propionic acid,

(S)-2-{[3′-(4-Chloro-2,5-dimethyl-benzenesulfonylamino)-3,5-dimethyl-biphenyl-4-ylmethyl]-amino}-propionic acid,

(R)-2-{[3′-(4-Chloro-2,5-dimethyl-benzenesulfonylamino)-3,5-dimethyl-biphenyl-4-ylmethyl]-amino}-propionic acid,

(S)-2-{[3′-(4-Chloro-2,5-dimethyl-benzenesulfonylamino)-3,5-dimethyl-biphenyl-4-ylmethyl]-methyl-amino}-propionic acid,

(R)-2-{[3′-(4-Chloro-2,5-dimethyl-benzenesulfonylamino)-3,5-dimethyl-biphenyl-4-ylmethyl]-amino}-propionic acid,

1-[3′-(4-Chloro-2,5-dimethyl-benzenesulfonylamino)-biphenyl-4-ylmethyl]-azetidine-3-carboxylic acid,

1-[3′-(4-Chloro-2,5-dimethyl-benzenesulfonylamino)-3-methyl-biphenyl-4-ylmethyl]-azetidine-3-carboxylic acid,

4-[3′-(4-Chloro-2,5-dimethyl-benzenesulfonylamino)-biphenyl-4-ylmethyl]-morpholine-3-carboxylic acid,

(2S,3S)-1-[3′-(4-Chloro-2,5-dimethyl-benzenesulfonylamino)-biphenyl-4-ylmethyl]-3-hydroxy-pyrrolidine-2-carboxylic acid,

(2S,4R)-1-[3′-(4-Chloro-2,5-dimethyl-benzenesulfonylamino)-biphenyl-4-ylmethyl]-4-hydroxy-pyrrolidine-2-carboxylic acid.

The compounds of the invention in free form or in pharmaceutically acceptable salt or ester form, in particular the compounds of formula I and/or a pharmaceutically acceptable salt thereof, exhibit valuable pharmacological properties, e.g. as S1P receptor modulators, especially S1P1 modulators, in particular S1P1 receptor antagonists, and are therefore indicated for therapy, especially those described in more detail hereinbelow.

Accordingly, the invention in a second aspect provides a compound as described above or a pharmaceutically-acceptable and -cleavable ester, or acid or amine addition salt thereof for use as a pharmaceutical.

The invention in a third aspect provides the use of a compound as described above or a pharmaceutically-acceptable and -cleavable ester, or acid addition salt thereof in the manufacture of a medicament for the treatment of a disease or disorder mediated by lymphocytes interactions.

The invention in a fourth aspect provides the use of a compound as described above or a pharmaceutically-acceptable and -cleavable ester, or acid addition salt thereof for the treatment of a disease or disorder mediated by lymphocytes interactions.

The invention in a fifth aspect provides a method of treatment of a disease or disorder mediated by lymphocytes interactions, e.g. as described hereinbelow, comprising administering an effective amount of a compound as described above or a pharmaceutically-acceptable and -cleavable ester, or acid addition salt thereof to a patient in need of such treatment.

The invention in a sixth aspect provides a pharmaceutical composition comprising a compound as described above or a pharmaceutically-acceptable and -cleavable ester, or acid addition salt thereof in association with a pharmaceutically acceptable excipient, diluent or carrier.

In a seventh aspect the invention provides a process for preparing a compound of formula (I) in free or salt form, comprising
a) For compounds of formula (I) wherein R1 and R2 taken together are O, the step of coupling a carboxylic acid of formula (IV) with an optionally protected amine of formula (V) or a salt thereof using standard coupling reagents, e.g. TBTU or HATU, and a base, e.g. Hünig's base or triethyl amine, followed by an optional deprotection step:

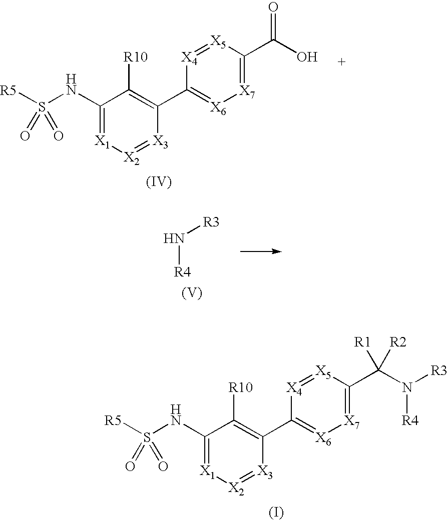

b) For compounds of formula (I) wherein R1 and R2 are both H, the step of reacting an aldehyde of formula (VI) with an optionally protected amine of formula (V) or a salt thereof under standard reductive amination conditions using standard reducing agents e.g. sodium triacetoxyborohydride or sodium cyanoborohydride, followed by an optional deprotection step:

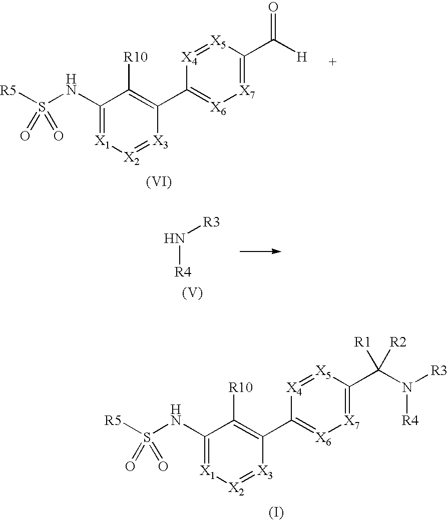

c) For compounds of formula (I) wherein one of R1 or R2 is alkyl, or R1 and R2 taken together are O, the step of reacting an optionally protected aniline of formula (VII) with a sulfonyl chloride of formula (VIII) in the presence of a base, e.g. pyridine or triethyl amine, followed by an optional deprotection step:

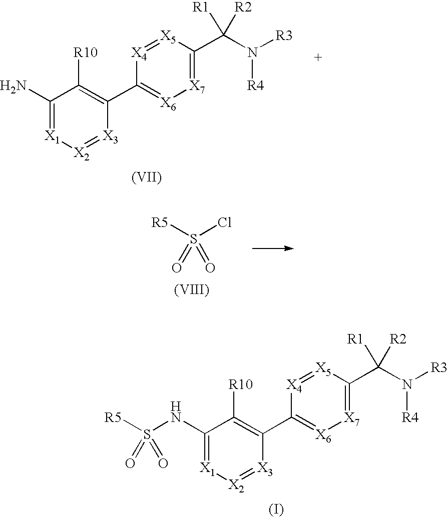

d) For compounds of formula (I) wherein one optional substituent on R3 is COOH, the step of reacting a polymer-bound aniline of formula (IX) with a sulfonyl chloride of formula (VIII) in the presence of a base, e.g. pyridine or DMAP, followed by acidic cleavage from the polymer:

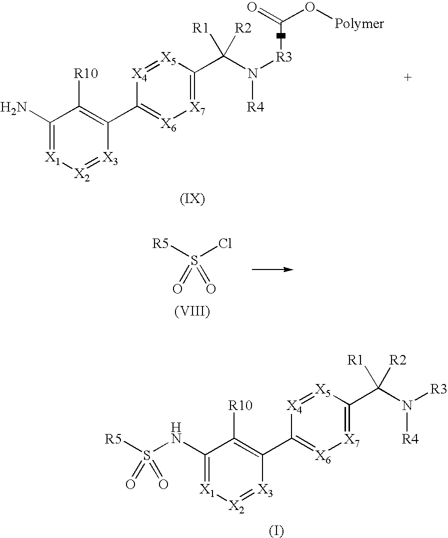

The compounds of formula (I) in free form may be converted into salt forms in conventional manner and vice-versa.

The compounds of the invention can be recovered from the reaction mixture and purified in conventional manner. Isomers, such as enantiomers, may be obtained in conventional manner, e.g. by fractional crystallization typically using chiral auxiliaries or optionally by separation involving chiral phases or by asymmetric synthesis from corresponding asymmetrically substituted, e.g. optically active starting materials.

In an eighth aspect the invention provides a combination of a compound as described above and an active agent selected from: an immunosuppressive or immunomodulating agent, anti-inflammatory agent, chemotherapeutic agent, calcineurin inhibitor, mTOR inhibitor, corticosteroid; PKC inhibitor, JAK3 kinase inhibitor, immunosuppressive monoclonal antibody, adhesion molecule inhibitor, or an anti-infectious agent.

The following Examples are illustrative of the invention:

### EXPERIMENTAL SECTION

### ABBREVIATIONS

AcOH: Acetic acid

BOC: t-Butyloxycarbonyl

DCE: Dichloroethane

DCM: Dichloromethane

DIPEA: Ethyl-diisopropyl-amine, Hünig's base, DIEA

DMAP: Dimethyl-pyridin-4-yl-amine

DMA: N,N-Dimethyl-acetamide

DME: 1,2-Dimethoxy-ethane

DMF: N,N-Dimethyl formamide

EDC (3-Dimethylamino-propyl)-ethyl-carbodiimide hydrochloride

Ether: Ethoxy-ethane

EtOAc: Acetic acid ethyl ester

EtOH: Ethanol

Fmoc: (9H-Fluoren-9-yl)-methoxycarbonyl

HATU: O-(7-Azabenzotriazol-1-yl)-N,N,N′N′-tetramethyluronium hexafluorophosphate

HOBt Benzotriazol-1-ol

LAH: Lithium aluminumhydride

MeOH: Methanol

Pd/C: Palladium on carbon

TBTU: O-(1H-Benzotriazol-1-yl)-N,N,N′,N′-tetramethyluronium tetrafluoroborate

TFA: Trifluoro-acetic acid

THF: Tetrahydrofuran

rt: Retention time

1H-NMR spectra are recorded on a Varian Gemini 400 MHz NMR spectrometer. Significant peaks are tabulated in the order: multiplicity (s, singlet; d, doublet; t, triplet; q, quartet; m, multiplet; br, broad) and number of protons. Electron Spray Ionization (ESI) mass spectra are recorded on a Hewlett Packard 5989A mass spectrometer. Mass spectrometry results are reported as the ratio of mass over charge. The following HPLC methods are used to purify and characterize the products.

Method A (Preparative): Method507509: Preparative HPLC Waters preparative HPLC instrument. Column: Waters Atlantis™ dC18, 100×30 mm 5 μm, reverse phase. Eluent A: water, 0.1% trifluoroacetic acid; B: acetonitrile. Flow rate: 30 ml/min. Detection: Photodiode Array Detector. Method: 5% B in A isocratic over 1.0 min, then gradient 5-100% B in A over 14 min, then isocratic 100% B in A over 1.5 min.

Method B: Method507.102

Waters 2795 Alliance HT instrument. Column: XTerra MS C18, 50×4.6 mm, 5 μm, reverse phase. Eluent A: water, 0.1% trifluoroacetic acid; B: acetonitrile, 0.1% trifluoroacetic acid. Flow rate: 2 ml/min. Detection: Photodiode Array Detector, Micromass ZQ, ELSD. Method: gradient 5-100% B in A over 8 min.

Method C: Method507.102Short

Waters 2795 Alliance HT instrument. Column: SunFire C18 20×4.6 mm, 3.5 μm, reverse phase. Eluent A: water, 0.1% trifluoroacetic acid; B: acetonitrile, 0.1% trifluoroacetic acid. Flow rate: 3 ml/min. Detection: Photodiode Array Detector, Micromass ZQ, ELSD. Method: gradient 5-100% B in A over 4 min.

Method D: Method507.701:

Waters 2795 Alliance HT instrument. Column: Macherey-Nagel C-18, Nucleosil, 70×4.6 mm, 3 μm, reverse phase. Eluent A: water, 0.05% trifluoroacetic acid; B: acetonitrile, 0.05% trifluoroacetic acid. Flow rate: 1.4 ml/min. Detection: Photodiode Array Detector, Mass spectrometer. Method: gradient 5-95% B in A over 8 min.

Method E: Standard-4.5 min-215 nm:

Merck Hitachi LaChrom instrument. Column: Interchim Modulo Cart QS Uptisphere 3 μm ODB, 50×4.6 mm, reverse phase. Eluent A: water, 0.1% trifluoroacetic acid; B: acetonitrile, 0.1% trifluoroacetic acid. Flow rate: 1.8 ml/min. Detection: UV (215 nm). Method: 5% B in A isocratic over 0.5 min, then gradient 10-95% B in A over 2 min, then isocratic 95% B in A over 1.4 min.

All reagents, starting materials and intermediates utilized in these examples are available from commercial sources or are readily prepared by methods known to those skilled in the art.

### Synthesis of Benzamide Derivatives

Agents of the invention may be prepared on solid support or in solution or by a combination of both techniques.

Synthesis on Solid Support

An illustrative example for a reaction sequence on solid support is shown in Reaction Scheme 1 below. The protected (e.g. FMOC) amino acid is conveniently attached through its carboxyl group to the solid support. Cleavage of the protecting group, amidation with a protected biaryl acid, cleavage of the protecting group, sulfonamidation with a sulfonyl chloride and, finally, acidic cleavage from the resin yields the desired products which may be further modified by standard chemical transformations in solution.

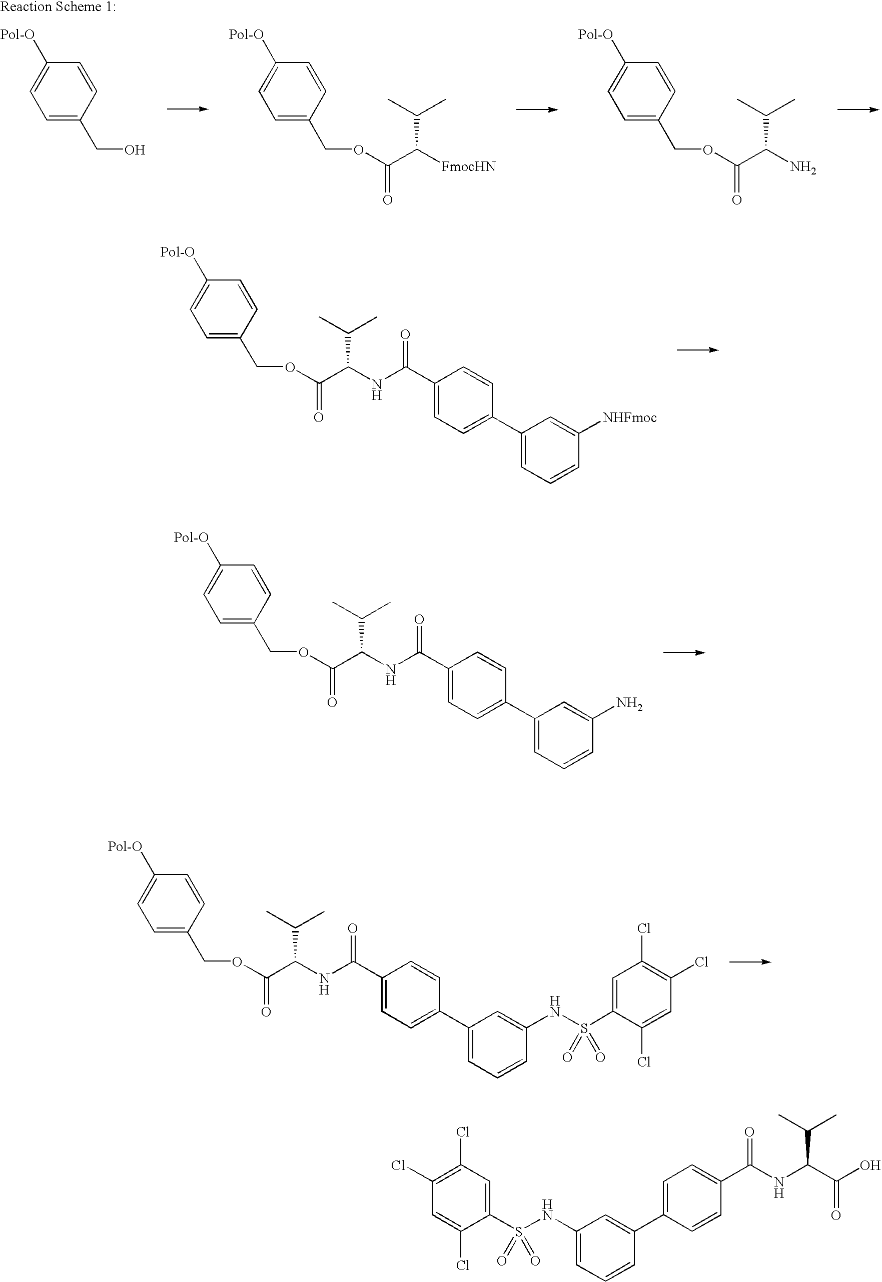

### EXAMPLE 1

### (S)-3-Methyl-2-{[3′-(2,4,5-trichloro-benzenesulfonylamino)-biphenyl-4-carbonyl]-amino}-butyric acid

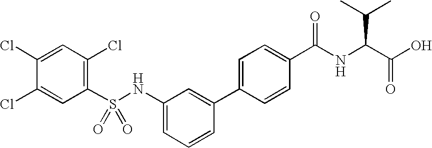

### (1) (S)-2-(9H-Fluoren-9-ylmethoxycarbonylamino)-3-methyl-butyric acid 4-methoxy-benzylpolystyryl ester (1)

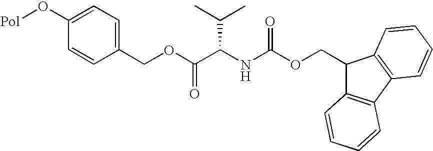

To a suspension of Wang Resin^{ref} ( 5.0 g, loading 1.8 mmol/g, 9.0 mmol) is added a solution of N-L-Fmoc-valine (9.2 g, 27.0 mmol) in 1/1 DMA/THF (42 ml). The resulting slurry is shaken for 20 minutes at room temperature on an orbital shaker before the addition of 2,6-dichlorobenzoyl chloride (1.87 ml, 27.0 mmol) and pyridine (3.23 ml, 45.0 mmol). Stirring is resumed for 18 hours. After that time, the title resin 1 is drained and washed successively with DMA, MeOH, and DCM and dried under vacuum.

### (2) (S)-2-Amino-3-methyl-butyric acid 4-benzyloxy-polystyryl ester (2)

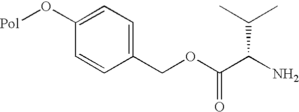

The resin 1 obtained in step 1 (9.0 mmol) is suspended in a mixture of piperidine and DMA (1/4, 42 ml) and shaken on an orbital shaker for 20 minutes before draining and washing with the above solution. This procedure is repeated one additional time before washing successively with DMA, MeOH, and DCM. The title resin 2 is then dried under vacuum.

### (3) (S)-2-{[3′-(9H-Fluoren-9-ylmethoxycarbonylamino)-biphenyl-4-carbonyl]-amino}-3-methyl-butyric acid 4-benzyloxy-polystyryl ester (3)

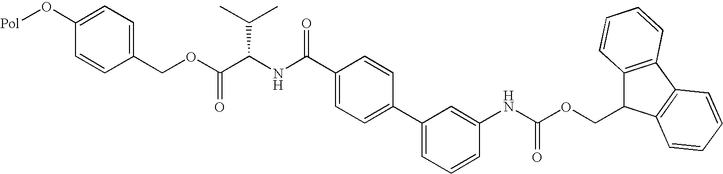

The resin 2 (3.6 mmol of bound species) obtained in step 2 is treated with a preformed solution of HATU (4.2 g, 10.8 mmol), DIPEA (3.77 ml, 21.6 mmol) and 3′-(9H-Fluoren-9-ylmethoxycarbonylamino)-biphenyl-4-carboxylic acid (4.75 g, 10.8 mmol) in NMP (36 ml) for 2 hours at 60° C. After that time, the resin is drained and washed successively with DMA, MeOH, and DCM to give the title resin 3.

### (4) (S)-2-[(3′-Amino-biphenyl-4-carbonyl)-amino]-3-methyl-pentanoic acid 4-benzyloxy-polystyryl ester (4)

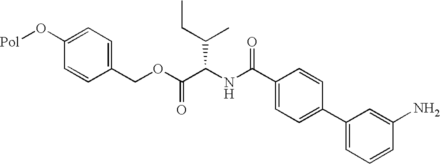

The resin 3 obtained in step 3 (3.6 mmol of bound species) is suspended in a mixture of piperidine and DMA (1/4, 36 ml) and shaken on an orbital shaker for 20 minutes before draining and washing with the above solution. This procedure is repeated one additional time before washing successively with DMA, MeOH, and DCM. The title resin 4 is then dried under vacuum.

### (5) (S)-3-Methyl-2-{[3′-(2,4,5-trichloro-benzenesulfonylamino)-biphenyl-4-carbonyl]-amino}-butyric acid 4-benzyloxy-polystyryl ester (5)

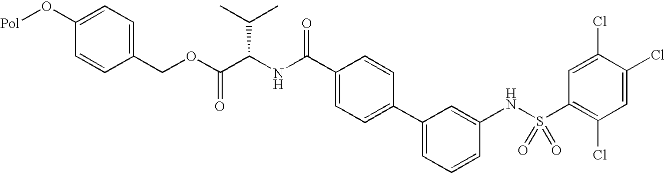

The resin 4 (0.18 mmol of bound species) obtained in step 4 is treated with a preformed solution of pyridine (516 μl, 7.20 mmol), DMAP (20.2 mg, 0.16 mmol), and 2,4,5-trichlorobenzenesulfonyl chloride (509 mg, 1.8 mmol) in DCE (2 ml) and shaken for one hour at room temperature on an orbital shaker. The resin is then washed successively with DMA, MeOH, and DCM and thoroughly dried under vacuum to give the title resin 5.

### (6) (S)-3-Methyl-2-{[3′-(2,4,5-trichloro-benzenesulfonylamino)-biphenyl-4-carbonyl]-amino}-butyric acid

The resin 5 (0.18 mmol of bound species) is treated with a 1/1 mixture of TFA and DCM (2 ml) for one hour at room temperature. The resin is drained and washed with DCM (3 times 2 ml). The combined organic phases are then concentrated, taken up in a minimum of methanol and submitted to purification by AP-RP-HPLC (Method A). The product-containing fractions are lyophilized to give the title compound Example 1 as a white powder. HPLC rt=6.32 min (Method D), MS (ESI): 554-557 [M+H]⁺.

1H-NMR (DMSO-d6): δ (ppm) 12.58 (br s, 1H), 10.99 (br s, 1H), 8.46 (d, 1H), 8.22 (s, 1H), 8.09 (s, 1H), 7.96 (d, 2H), 7.59 (d, 2H), 7.37 (m, 3H), 7.13 (m, 1H), 4.30 (m, 1H), 2.21 (m, 1H), 0.99 (m, 6H).

### EXAMPLE 2

### (S)-2-{[3′-(3,4-Dichloro-benzenesulfonylamino)-biphenyl-4-carbonyl]-amino}-3-methyl-butyric acid

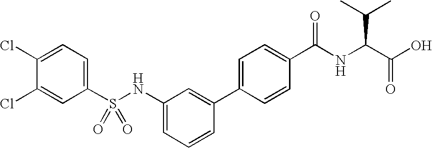

This compound is synthesized using the same synthetic sequence as Example 1 using 3,4-dichlorobenzenesulfonyl chloride instead of 2,4,5-trichlorobenzenesulfonyl chloride in step 5. HPLC rt=4.93 min (Method B), MS (ESI): 520-522 [M+H]⁺.

1H-NMR (DMSO-d6): δ (ppm) 12.17 (br s, 1H), 11.02 (br s, 1H), 8.44 (d, 1H), 7.96 (m, 3H), 7.84 (d, 1H), 7.71 (d, 1H), 7.61 (d, 2H), 7.37 (m, 3H), 7.13 (m, 1H), 4.30 (m, 1H), 2.21 (m, 1H), 0.98 (m, 6H).

### EXAMPLE 3

### (S)-3-Methyl-2-{[3′-(naphthalene-2-sulfonylamino)-biphenyl-4-carbonyl]-amino}-butyric acid

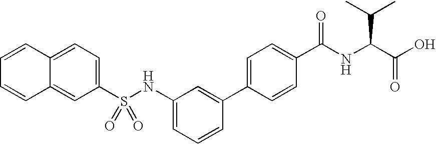

This compound is synthesized using the same synthetic sequence as Example 1 using naphthalene-2-sulfonyl chloride instead of 2,4,5-trichlorobenzenesulfonyl chloride in step 5. HPLC rt=5.84 min (Method D), MS (ESI): 503 [M+H]⁺.

1H-NMR (DMSO-d6): δ (ppm) 12.57 (br s, 1H), 10.54 (br s, 1H), 8.49 (s, 1H), 8.42 (d, 1H), 8.14 (d, 1H), 7.98 (d, 1H), 7.92 (d, 2H), 7.75-7.10 (m, 10H), 4.29 (m, 1H), 2.20 (m, 1H), 0.98 (m, 6H).

### EXAMPLE 4

### {[3′-(4-Chloro-benzenesulfonylamino)-biphenyl-4-carbonyl]-amino}-acetic acid

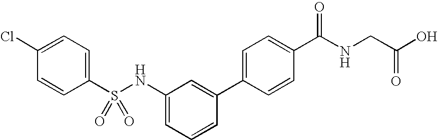

This compound is synthesized using the same synthetic sequence as Example 1 using 4-chlorobenzenesulfonyl chloride instead of 2,4,5-trichlorobenzenesulfonyl chloride in step 5. HPLC rt=3.28 min (Method E), MS (ESI): 445-447 [M+H]⁺.

1H-NMR (DMSO-d6): δ (ppm) 10.49 (s, 1H), 8.87 (t, 1H), 7.94 (d, 2H), 7.78 (d, 2H), 7.62 (m, 4H), 7.38 (m, 3H), 7.11 (d, 1H), 3.94 (d, 2H).

### EXAMPLE 5

### (S)-2-{[3′-(5-Chloro-naphthalene-2-sulfonylamino)-biphenyl-4-carbonyl]-amino}-3-methyl-butyric acid

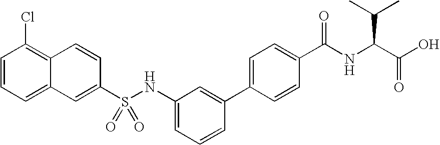

This compound is synthesized using the same synthetic sequence as Example 1 using 5-Chloro-naphthalene-2-sulfonyl chloride instead of 2,4,5-trichlorobenzenesulfonyl chloride in step 5. HPLC rt=4.96 min (Method B), MS (ESI): 536-538 [M+H]⁺.

1H-NMR (DMSO-d6): δ (ppm) 12.56 (br s, 1H), 10.62 (s, 1H), 8.60 (s, 1H), 7.44 (d, 1H), 8.34 (d, 1H), 8.18 (d, 1H), 7.98-7.80 (m, 4H), 7.62 (t, 1H), 7.55 (d, 2H), 7.42 (s, 1H), 7.13-7.33 (m, 3H), 4.30 (m, 1H), 2.20 (m, 1H), 0.98 (t, 6H).

### EXAMPLE 6

### (S)-2-{[3′-(4-Chloro-3-methyl-benzenesulfonylamino)-biphenyl-4-carbonyl]-amino}-3-methyl-butyric acid

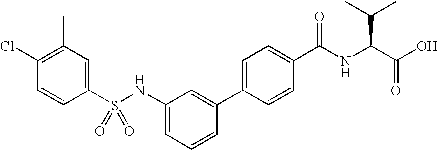

This compound is synthesized using the same synthetic sequence as Example 1 using 4-Chloro-3-methylbenzenesulfonyl chloride instead of 2,4,5-trichlorobenzenesulfonyl chloride in step 5. HPLC rt=4.70 min (Method B), MS (ESI): 501-503 [M+H]⁺.

1H-NMR (DMSO-d6): δ (ppm) 12.60 (br s, 1H), 10.46 (s, 1H), 8.45 (d, 1H), 7.96 (d, 2H), 7.79 (s, 1H), 7.61 (m, 2H), 7.38-7.10 (m, 4H), 4.31 (m, 1H), 2.36 (s, 3H), 2.20 (m, 1H), 0.99 (t, 6H).

### EXAMPLE 7

### (S)-2-{[3′-(2,4-Dimethyl-benzenesulfonylamino)-biphenyl-4-carbonyl]-amino}-3-methyl-butyric acid

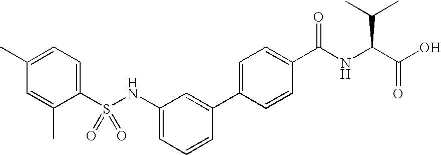

This compound is synthesized using the same synthetic sequence as Example 1 using 2,4-dimethylbenzenesulfonyl chloride instead of 2,4,5-trichlorobenzenesulfonyl chloride in step 5. HPLC rt=4.51 min (Method B), MS (ESI): 481 [M+H]⁺.

1H-NMR (DMSO-d6): δ (ppm) 12.59 (br s, 1H), 10.48 (s, 1H), 8.44 (d, 1H), 7.95 (d, 2H), 7.83 (d, 1H), 7.56 (d, 2H), 7.40-7.05 (m, 6H), 4.30 (m, 1H), 2.57 (s, 3H), 2.29 (s, 3H), 2.20 (m, 1H), 0.99 (t, 6H).

### EXAMPLE 8

### (S)-2-{[3′-(2,4-Dichloro-5-methyl-benzenesulfonylamino)-biphenyl-4-carbonyl]-amino}-3-methyl-butyric acid

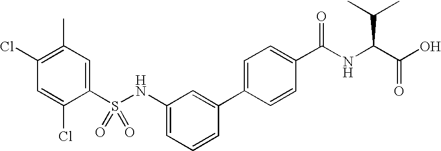

This compound is synthesized using the same synthetic sequence as Example 1 using 2,4-dichloro-5-methylbenzenesulfonyl chloride instead of 2,4,5-trichlorobenzenesulfonyl chloride in step 5. HPLC rt=4.93 min (Method B), MS (ESI): 534-536 [M+H]⁺.

1H-NMR (DMSO-d6): δ (ppm) 12.60 (br s, 1H), 10.80 (s, 1H), 8.45 (d, 1H), 8.12 (s, 1H), 7.96 (d, 2H), 7.79 (s, 1H), 7.57 (d, 2H), 7.39-7.04 (m, 4H), 4.31 (m, 1H), 2.38 (s, 3H), 2.21 (m, 1H), 0.99 (t, 6H).

### EXAMPLE 9

### (S)-2-{[3′-(2,5-Dichloro-3,6-dimethyl-benzenesulfonylamino)-biphenyl-4-carbonyl]-amino}-3-methyl-butyric acid

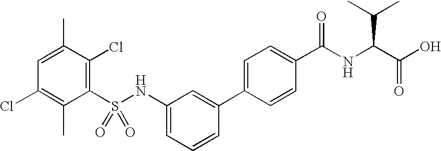

This compound is synthesized using the same synthetic sequence as Example 1 using 2,5-dichloro-3,6-dimethylbenzenesulfonyl chloride instead of 2,4,5-trichlorobenzenesulfonyl chloride in step 5. HPLC rt=5.10 min (Method B), MS (ESI): 549-551 [M+H]⁺.

1H-NMR (DMSO-d6): δ (ppm) 12.58 (br s, 1H), 10.81 (s, 1H), 8.46 (d, 1H), 7.96 (d, 1H), 7.78 (s, 1H), 7.53 (d, 2H), 7.36-7.04 (m, 4H), 4.30 (m, 1H), 2.73 (s, 3H), 2.32 (s, 3H), 2.20 (m, 1H), 0.99 (t, 6H).

### EXAMPLE 10

### (S)-2-{[3′-(4-Chloro-3-trifluoromethyl-benzenesulfonylamino)-biphenyl-4-carbonyl]-amino}-3-methyl-butyric acid

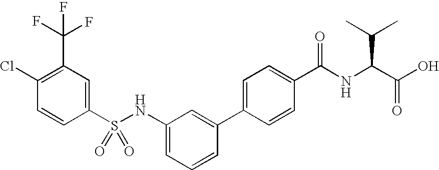

This compound is synthesized using the same synthetic sequence as Example 1 using 4-Chloro-3-trifluoromethyl-benzenesulfonyl chloride instead of 2,4,5-trichlorobenzenesulfonyl chloride in step 5. HPLC rt=4.93 min (Method B), MS (ESI): 554-557 [M+H]⁺.

1H-NMR (DMSO-d6): δ (ppm) 12.58 (br s, 1H), 10.60 (s, 1H), 8.46 (d, 1H), 8.10 (s, 1H), 8.05-7.90 (m, 4H), 7.59 (d, 2H), 7.37-7.45 (m, 3H), 7.12 (d, 1H), 4.30 (m, 1H), 2.21 (m, 1H), 0.99 (t, 6H).

### EXAMPLE 11

### (S)-3-Methyl-2-{[3′-(2,4,6-trimethyl-benzenesulfonylamino)-biphenyl-4-carbonyl]-amino}-butyric acid

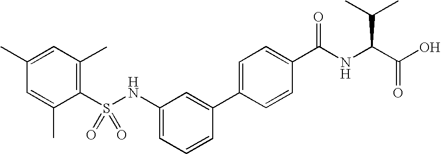

This compound is synthesized using the same synthetic sequence as Example 1 using 2,4,6-trimethylbenzenesulfonyl chloride instead of 2,4,5-trichlorobenzenesulfonyl chloride in step 5. HPLC rt=5.97 min (Method D), MS (ESI): 495 [M+H]⁺.

### EXAMPLE 12

### (S)-2-{[3′-(2,3-Dichloro-benzenesulfonylamino)-biphenyl-4-carbonyl]-amino}-3-methyl-butyric acid

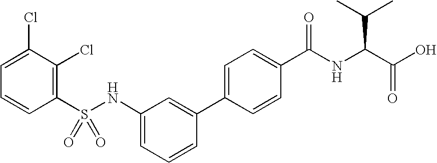

This compound is synthesized using the same synthetic sequence as Example 1 using 2,3-dichlorobenzenesulfonyl chloride instead of 2,4,5-trichlorobenzenesulfonyl chloride in step 5. HPLC rt=5.97 min (Method D), MS (ESI): 495 [M+H]⁺.

### EXAMPLE 13

### (S)-2-{[3′-(3-Chloro-2-methyl-benzenesulfonylamino)-biphenyl-4-carbonyl]-amino}-3-methyl-butyric acid

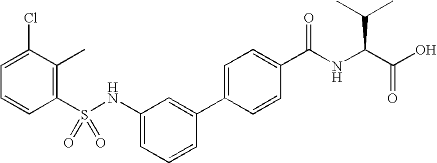

This compound is synthesized using the same synthetic sequence as Example 1 using 3-Chloro-2-methyl benzenesulfonyl chloride instead of 2,4,5-trichlorobenzenesulfonyl chloride in step 5. HPLC rt=5.93 min (Method D), MS (ESI): 500-502 [M+H]⁺.

### EXAMPLE 14

### (S)-3-Methyl-2-{[3′-(2-methyl-5-nitro-benzenesulfonylamino)-biphenyl-4-carbonyl]-amino}-butyric acid

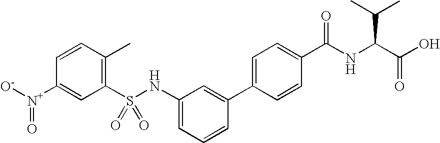

This compound is synthesized using the same synthetic sequence as Example 1 using 2-methyl-5-nitro-benzenesulfonyl chloride instead of 2,4,5-trichlorobenzenesulfonyl chloride in step 5. HPLC rt=5.62 min (Method D), MS (ESI): 512 [M+H]⁺.

### EXAMPLE 15

### (S)-2-{[3′-(4-Methoxy-2,3,6-trimethyl-benzenesulfonylamino)-biphenyl-4-carbonyl]-amino}-3-methyl-butyric acid

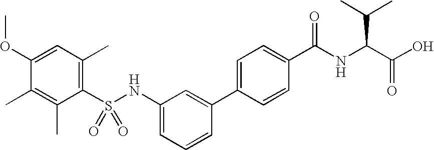

This compound is synthesized using the same synthetic sequence as Example 1 using 4-Methoxy-2,3,6-trimethylbenzenesulfonyl chloride instead of 2,4,5-trichlorobenzenesulfonyl chloride in step 5. HPLC rt=4.71 min (Method B), MS (ESI): 525 [M+H]⁺.

### EXAMPLE 16

### (S)-2-{[3′-(3,5-Dichloro-benzenesulfonylamino)-biphenyl-4-carbonyl]-amino}-3-methyl-butyric acid

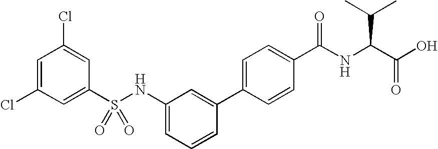

This compound is synthesized using the same synthetic sequence as Example 1 using 3,5-Dichloro-benzenebenzenesulfonyl chloride instead of 2,4,5-trichlorobenzenesulfonyl chloride in step 5. HPLC rt=4.88 min (Method B), MS (ESI): 520-522 [M+H]⁺.

### EXAMPLE 17

### (S)-2-{[3′-(2,4-Dichloro-benzenesulfonylamino)-biphenyl-4-carbonyl]-amino}-3-methyl-butyric acid

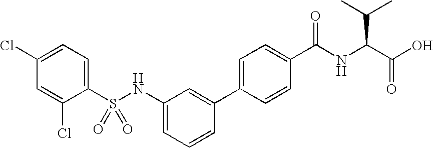

This compound is synthesized using the same synthetic sequence as Example 1 using 2,4-Dichloro-benzenebenzenesulfonyl chloride instead of 2,4,5-trichlorobenzenesulfonyl chloride in step 5. HPLC rt=4.68 min (Method B), MS (ESI): 520-522 [M+H]⁺.

### EXAMPLE 18

### (S)-3-Methyl-2-[(3′-pentamethylbenzenesulfonylamino-biphenyl-4-carbonyl)-amino]-butyric acid

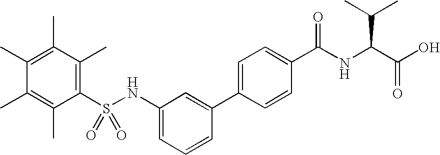

This compound is synthesized using the same synthetic sequence as Example 1 using pentamethylbenzenebenzenesulfonyl chloride instead of 2,4,5-trichlorobenzenesulfonyl chloride in step 5. HPLC rt=4.92 min (Method B), MS (ESI): 523 [M+H]⁺.

### EXAMPLE 19

### (S)-3-Methyl-2-{[3′-(2,3,5,6-tetramethyl-benzenesulfonylamino)-biphenyl-4-carbonyl]-amino}-butyric acid

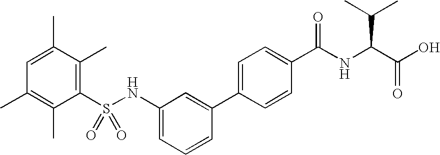

This compound is synthesized using the same synthetic sequence as Example 1 using 2,3,5,6-tetramethylbenzenebenzenesulfonyl chloride instead of 2,4,5-trichlorobenzenesulfonyl chloride in step 5. HPLC rt=4.82 min (Method B), MS (ESI): 509 [M+H]⁺.

### EXAMPLE 20

### (S)-2-{[3′-(2,5-Dimethyl-benzenesulfonylamino)-biphenyl-4-carbonyl]-amino}-3-methyl-butyric acid

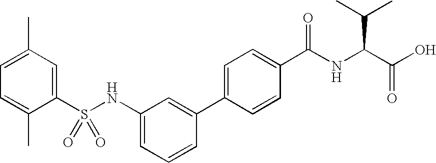

This compound is synthesized using the same synthetic sequence as Example 1 using 2,5-Dimethylbenzenebenzenesulfonyl chloride instead of 2,4,5-trichlorobenzenesulfonyl chloride in step 5. HPLC rt=4.50 min (Method B), MS (ESI): 481 [M+H]⁺.

### EXAMPLE 21

### (S)-2-{[3′-(4-Chloro-2,5-dimethyl-benzenesulfonylamino)-biphenyl-4-carbonyl]-amino}-3-methyl-pentanoic acid

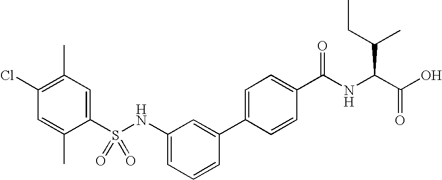

This compound is synthesized using the same synthetic sequence as Example 1 using N-Fmoc-L-isoleucine instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=5.30 min (Method B), MS (ESI): 529-531 [M+H]⁺.

1H-NMR (DMSO-d6): δ (ppm) 12.57 (br s, 1H), 10.59 (s, 1H), 8.47 (d, 1H), 7.94 (m, 3H), 7.56 (d, 2H), 7.47 (s, 1H), 7.34-7.08 (m, 4H), 4.35 (m, 1H), 2.55 (s, 3H), 2.35 (s, 3H), 1.97 (m, 1H), 1.53 (m, 1H), 1.29 (m, 1H), 0.95 (d, 3H), 0.89 (t, 3H).

### EXAMPLE 22

### (S)-2-{[3′-(4-Chloro-2,5-dimethyl-benzenesulfonylamino)-biphenyl-4-carbonyl]-amino}-3-methyl-butyric acid

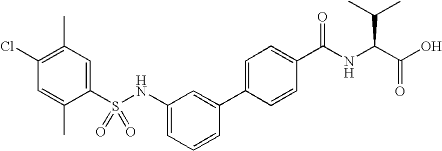

This compound is synthesized using the same synthetic sequence as Example 1 using 4-Chloro-2,5-dimethyl-benzenesulfonyl chloride instead of 2,4,5-trichlorobenzenesulfonyl chloride in step 5. HPLC rt=6.17 min (Method D), MS (ESI): 515-517 [M+H]⁺.

### EXAMPLE 23a

### {[3′-(4-Chloro-2,5-dimethyl-benzenesulfonylamino)-biphenyl-4-carbonyl]-amino}-acetic acid

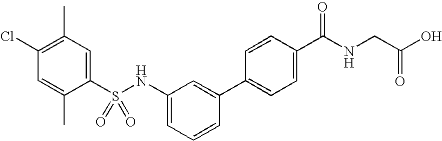

This compound is synthesized using the same synthetic sequence as Example 1 using N-Fmoc-glycine instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=4.48 min (Method B), MS (ESI): 472-474 [M+H]⁺.

### EXAMPLE 23b

### {[3′-(4-Chloro-2,5-dimethyl-benzenesulfonylamino)-biphenyl-4-carbonyl]-amino}-acetatemethyl-((2S,3R,4R,5R)-2,3,4,5,6-pentahydroxy-hexyl)-ammonium

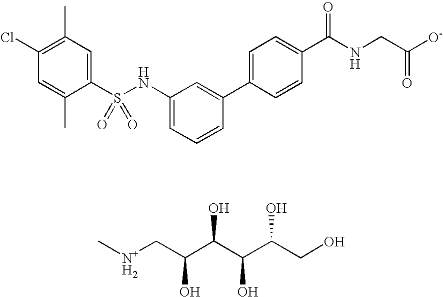

A solution of Example 23a (100 mg, 0.211 mmol) in 2.5 ml of MeOH is mixed with a solution of (2R,3R,4R,5S)-6-Methylamino-hexane-1,2,3,4,5-pentaol (N-methyl-D-glucamine, 41.3 mg, 0.211 mmol) in 2.5 ml of MeOH. The clear solution is filtered and evaporated to dryness to give a white foam. This is triturated with ether, filtered off and dried to give the title compound as white powder.

MS (ESI): 471-473 [M−H]⁻.

### EXAMPLE 24

### (R)-2-{[3′-(4-Chloro-2,5-dimethyl-benzenesulfonylamino)-biphenyl-4-carbonyl]-amino}-3-methyl-butyric acid

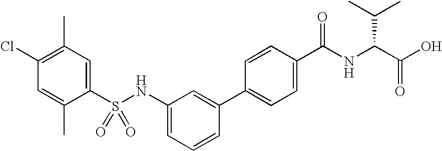

This compound is synthesized using the same synthetic sequence as Example 1 using N-Fmoc-D-valine instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=5.12 min (Method B), MS (ESI): 515-517 [M+H]⁺.

### EXAMPLE 25

### (S)-2-{[3′-(4-Chloro-2,5-dimethyl-benzenesulfonylamino)-biphenyl-4-carbonyl]-amino}-propionic acid

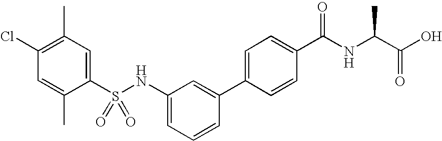

This compound is synthesized using the same synthetic sequence as Example 1 using N-Fmoc-L-alanine instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=3.51 min (Method E), MS (ESI): 487-489 [M+H]⁺.

### EXAMPLE 26

### (S)-2-{[3′-(4-Chloro-2,5-dimethyl-benzenesulfonylamino)-biphenyl-4-carbonyl]-amino}-3-phenyl-propionic acid

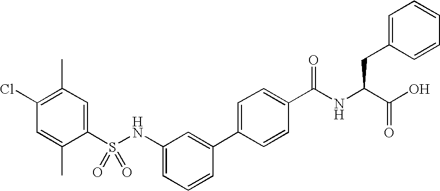

This compound is synthesized using the same synthetic sequence as Example 1 using N-Fmoc-L-phenylalanine instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=5.28 min (Method B), MS (ESI): 563-565 [M+H]⁺.

1H-NMR (DMSO-d6): δ (ppm) 12.76 (br s, 1H), 10.58 (s, 1H), 8.72 (d, 1H), 7.95 (s, 1H), 7.86 (d, 2H), 7.53 (d, 2H), 7.47 (s, 1H), 7.32-7.04 (m, 9H), 4.63 (m, 1H), 3.20 (m, 1H), 3.09 (m, 1H), 2.54 (s, 3H), 2.35 (s, 3H).

### EXAMPLE 27

### (S)-1-[3′-(4-Chloro-2,5-dimethyl-benzenesulfonylamino)-biphenyl-4-carbonyl]-pyrrolidine-2-carboxylic acid

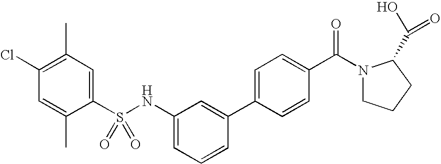

This compound is synthesized using the same synthetic sequence as Example 1 using N-Fmoc-L-proline instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=4.70 min (Method B), MS (ESI): 513-515 [M+H]⁺.

1H-NMR (DMSO-d6): δ (ppm) 12.55 (br s, 1H), 10.59 (s, 1H), 7.95 (s, 1H), 7.60 (d, 2H), 7.55 (d, 2H), 7.47 (s, 1H), 7.32-7.04 (m, 4H), 4.41 (m, 1H), 3.55 (m, 2H), 2.54 (s, 3H), 2.35 (s, 3H), 2.25 (m, 1H), 1.91 (m, 3H).

### EXAMPLE 28a

### (S)-2-{[3′-(4-Chloro-2,5-dimethyl-benzenesulfonylamino)-biphenyl-4-carbonyl]-amino}-3-hydroxy-propionic acid

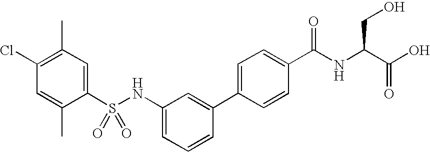

This compound is synthesized using the same synthetic sequence as Example 1 using N-Fmoc-O-^{t}Bu-L-serine instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=3.44 min (Method E), MS (ESI): 503-505 [M+H]⁺.

1H-NMR (DMSO-d6): δ (ppm) 12.72 (br s, 1H), 10.62 (s, 1H), 8.49 (d, 1H), 7.95 (m, 3H), 7.58 (d, 2H), 7.47 (s, 1H), 7.34 (m, 3H), 7.04 (m, 1H), 4.50 (m, 1H), 3.80 (m, 2H), 2.54 (s, 3H), 2.36 (s, 3H).

### EXAMPLE 28b

### (S)-2-{[3′-(4-Chloro-2,5-dimethyl-benzenesulfonylamino)-biphenyl-4-carbonyl]-amino}-3-hydroxy-propionate methyl-((2S,3R,4R,5R)-2,3,4,5,6-pentahydroxy-hexyl)-ammonium

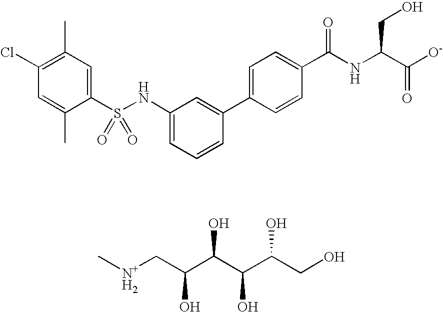

A solution of Example 28a (1 g, 2 mmol) in 20 ml of MeOH is mixed with a solution of (2R,3R,4R,5S)-6-Methylamino-hexane-1,2,3,4,5-pentaol (N-methyl-D-glucamine, 388 mg, 2 mmol) in 40 ml of MeOH. The clear solution is filtered and evaporated to dryness to give the title compound as white foam. This is triturated with ether, filtered off and dried to give the title compound as white powder.

### EXAMPLE 29

### {[3′-(4-Chloro-2,5-dimethyl-benzenesulfonylamino)-biphenyl-4-carbonyl]-methyl-amino}-acetic acid

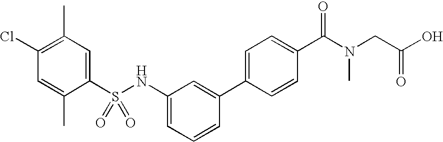

This compound is synthesized using the same synthetic sequence as Example 1 using N-Fmoc-L-sarcosine instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=3.61 min (Method E), MS (ESI): 487-489 [M+H]⁺.

1H-NMR (DMSO-d6): δ (ppm) 12.82 (br s, 1H), 10.59 (s, 1H), 7.94 (s, 1H), 7.80-7.32 (m, 9H), 7.07 (m, 1H), 4.16 (s, 2H), 2.99 (s, 3H), 2.54 (s, 3H), 2.34 (s, 3H).

### EXAMPLE 30

### 3-{[3′-(4-Chloro-2,5-dimethyl-benzenesulfonylamino)-biphenyl-4-carbonyl]-amino}-propionic acid

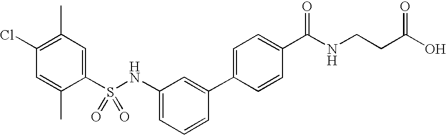

This compound is synthesized using the same synthetic sequence as Example 1 using 3-(9H-Fluoren-9-ylmethoxycarbonylamino)-propionic acid instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=4.37 min (Method B), MS (ESI): 486-488 [M+H]⁺.

1H-NMR (DMSO-d6): δ (ppm) 12.17 (br s, 1H), 10.59 (s, 1H), 8.57 (t, 1H), 7.95 (s, 1H), 7.90 (d, 2H), 7.54 (d, 2H), 7.47 (s, 1H), 7.32-7.06 (m, 3H), 3.47 (m, 2H), 2.54 (s, 3H), 2.53 (m, 2H), 2.32 (s, 3H).

### EXAMPLE 31

### (S)-3-{[3′-(4-Chloro-2,5-dimethyl-benzenesulfonylamino)-biphenyl-4-carbonyl]-amino}-butyric acid

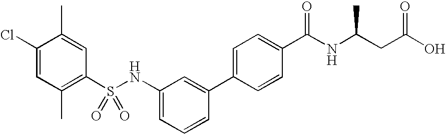

This compound is synthesized using the same synthetic sequence as Example 1 using (S)-3-(9H-Fluoren-9-ylmethoxycarbonylamino)-butyric acid instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=4.52 min (Method B), MS (ESI): 500-502 [M+H]⁺.

### EXAMPLE 32

### (S)-2-{[3′-(4-Chloro-2,5-dimethyl-benzenesulfonylamino)-biphenyl-4-carbonyl]-amino}-butyric acid

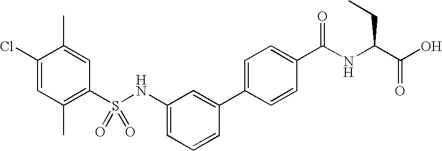

This compound is synthesized using the same synthetic sequence as Example 1 using (S)-2-(9H-Fluoren-9-ylmethoxycarbonylamino)-butyric acid instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=4.72 min (Method B), MS (ESI): 500-502 [M+H]⁺.

1H-NMR (DMSO-d6): δ (ppm) 12.53 (br s, 1H), 10.60 (s, 1H), 8.60 (d, 1H), 7.96 (m, 3H), 7.56 (d, 2H), 7.47 (s, 1H), 7.34-7.08 (m, 4H), 4.31 (m, 1H), 2.55 (s, 3H), 2.36 (s, 3H), 1.58 (m, 2H), 0.97 (t, 3H).

### EXAMPLE 33

### (R)-3-{[3′-(4-Chloro-2,5-dimethyl-benzenesulfonylamino)-biphenyl-4-carbonyl]-amino}-4-methyl-pentanoic acid

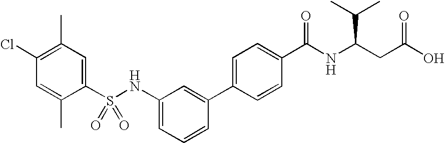

This compound is synthesized using the same synthetic sequence as Example 1 using (R)-3-(9H-Fluoren-9-ylmethoxycarbonylamino)-4-methyl-pentanoic acid instead of N-Fmoc-L-valine in step 1 and 4-Chloro-2,5-dimethyl-benzenesulfonyl chloride instead of 2,4,5-trichlorobenzenesulfonyl chloride in step 5. HPLC rt=4.89 min (Method B), MS (ESI): 529-531 [M+H]⁺.

1H-NMR (DMSO-d6): δ (ppm) 12.04 (br s, 1H), 10.59 (s, 1H), 8.20 (d, 1H), 7.95 (s, 1H), 7.88 (d, 2H), 7.54 (d, 2H), 7.47 (s, 1H), 7.33-7.07 (m, 4H), 4.22 (m, 1H), 2.54 (s, 3H), 2.48 (m, 2H), 2.35 (s, 3H), 1.86 (m, 1H), 0.90 (t, 6H).

### EXAMPLE 34

### 2-{[3′-(4-Chloro-2,5-dimethyl-benzenesulfonylamino)-biphenyl-4-carbonyl]-amino}-2-methyl-propionic acid

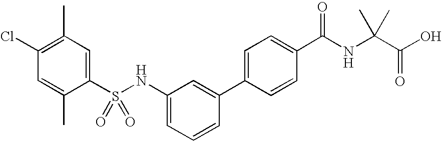

### (1) 2-(9H-Fluoren-9-ylmethoxycarbonylamino)-2-methyl-propionic acid (2-chloro-phenyl)-(4-polystyryl-phenyl)-phenyl-methyl ester (6)

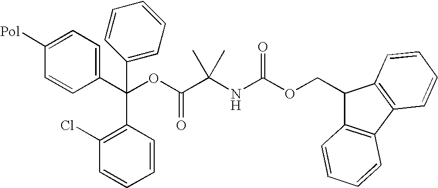

To a suspension of 2-chlorotrityl chloride resin^{ref} (150 mg, loading 1.05 mmol/g, 0.16 mmol) is added a preformed solution of 2-(9H-Fluoren-9-ylmethoxycarbonylamino)-2-methyl-propionic acid (155 mg, 0.47 mmol) and DIPEA (165 μl, 0.96 mmol) in DCM (1.6 ml). The resulting slurry is shaken for 18 hours at room temperature on an orbital shaker. After that time, the title resin 6 is drained and washed successively with DMA, MeOH, and DCM and dried under vacuum.

### (2) 2-{[3′-(4-Chloro-2,5-dimethyl-benzenesulfonylamino)-biphenyl-4-carbonyl]-amino}-2-methyl-propionic acid

The resin 6 is treated as described in steps 2 to 5 of Example 1, but using 4-chloro-2,5-dimethyl-benzenesulfonyl chloride instead of 2,4,5-trichlorobenzenesulfonyl chloride in step 5. The resulting resin is treated with a 1/1 mixture of TFA and DCM (2 ml) for one hour at room temperature, drained and washed with DCM (3 times 2 ml). The combined organic phases are then concentrated, taken up in a minimum of methanol and submitted to purification by AP-RP-HPLC (Method A). The product-containing fractions are lyophilized to give the title compound Example 34 as a white powder. HPLC rt=4.63 min (Method B), MS (ESI): 501-503 [M+H]⁺.

1H-NMR (DMSO-d6): δ (ppm) 12.15 (br s, 1H), 10.59 (s, 1H), 8.47 (s, 1H), 7.95 (s, 1H), 7.92 (d, 2H), 7.55 (d, 2H), 7.47 (s, 1H), 7.33-7.04 (m, 4H), 2.54 (s, 3H), 2.35 (s, 3H), 1.47 (s, 6H).

### EXAMPLE 35

### (S)-3-{[3′-(4-Chloro-2,5-dimethyl-benzenesulfonylamino)-biphenyl-4-carbonyl]-amino}-4-phenyl-butyric acid

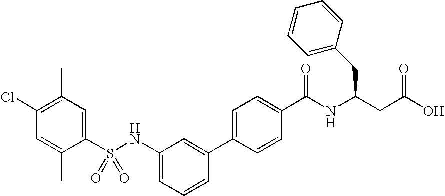

This compound is synthesized using the same synthetic sequence as Example 34 using (S)-3-(9H-Fluoren-9-ylmethoxycarbonylamino)-4-phenyl-butyric acid instead of 2-(9H-Fluoren-9-ylmethoxycarbonylamino)-2-methyl-propionic acid in step 1. HPLC rt=5.11 min (Method B), MS (ESI): 577-579 [M+H]⁺.

1H-NMR (DMSO-d6): δ (ppm) 12.12 (br s, 1H), 10.70 (br s, 1H), 8.39 (d, 1H), 7.95 (s, 1H), 7.82 (d, 2H), 7.52 (d, 2H), 7.47 (s, 1H), 7.40-7.05 (m, 9H), 4.50 (m, 1H), 2.88 (m, 2H), 2.54 (s, 3H), 2.52 (m, 2H), 2.35 (s, 3H).

### EXAMPLE 36

### (R)-3-{[3′-(4-Chloro-2,5-dimethyl-benzenesulfonylamino)-biphenyl-4-carbonyl]-amino}-3-phenyl-propionic acid

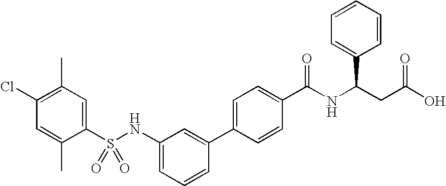

This compound is synthesized using the same synthetic sequence as Example 34 using (S)-3-(9H-Fluoren-9-ylmethoxycarbonylamino)-3-phenyl-propanoic acid instead of 2-(9H-Fluoren-9-ylmethoxycarbonylamino)-2-methyl-propionic acid in step 1. HPLC rt=5.00 min (Method B), MS (ESI): 563-565 [M+H]⁺.

1H-NMR (DMSO-d6): δ (ppm) 12.23 (br s, 1H), 10.61 (br s, 1H), 8.93 (d, 1H), 7.95 (m, 3H), 7.56 (d, 2H), 7.46 (s, 1H), 7.41-7.05 (m, 9H), 5.45 (m, 1H), 2.92 (m, 1H), 2.79 (m, 1H), 2.54 (s, 3H), 2.35 (s, 3H).

### EXAMPLE 37

### 3′-(4-Chloro-2,5-dimethyl-benzenesulfonylamino)-biphenyl-4-carboxylic acid (3-methoxy-propyl)-amide

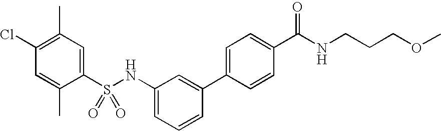

### (1) (4-polystyryloxy-2,6-dimethoxy-benzyl)-(3-methoxy-propyl)-amine (7)

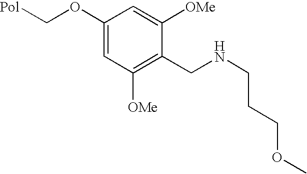

Commercially available 2-(3,5-dimethoxy-4-formylphenoxy)ethoxymethyl polystyrene (25 g, 1 mmol/g, 25 mmol) is washed 4 times with a 10/3 mixture of DCE and trimethoxy-methane (150 ml). The resin is then suspended in the above 10/3 mixture of DCE and TRIMETHOXY-METHANE (150 ml) again and treated with 1-amino-3-methoxy-propane (11.1 g, 125 mmol). The resulting slurry is shaken on an orbital shaker at room temperature for 16 hours before the resin is drained and washed successively with DMA, THF and DCM. A preformed solution of MeOH (5.1 ml, 125 mmol), AcOH (7.2 ml, 125 mmol) and borane-pyridine complex (125 mmol) in DCM is then added to the resin and shaking is resumed for 4 hours at room temperature. The resin is then finally drained, washed successively with DMA, AcOH/DMA (1/19), DMA, THF/H₂O (9/1), THF, DCM, MeOH, THF, MeOH. The title resin 7 is finally thoroughly dried under vacuum to a constant weight.

### (2) {4′-[(4-polystyryloxy-2,6-dimethoxy-benzyl)-(3-methoxy-propyl)-carbamoyl]-biphenyl-3-yl}-carbamic acid 9H-fluoren-9-ylmethyl ester (8)

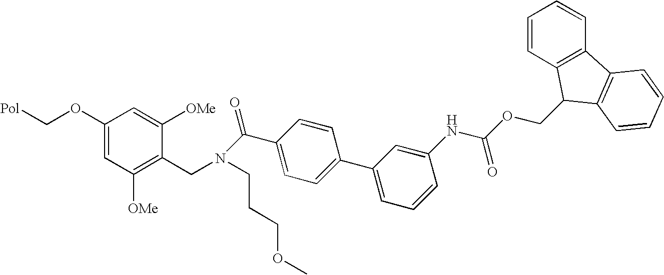

This step is carried out in the same manner as for step 3 of Example 1.

### (3) 3′-Amino-biphenyl-4-carboxylic acid (4-polystyryloxy-2,6-dimethoxy-benzyl)-(3-methoxy-propyl)-amide (9)

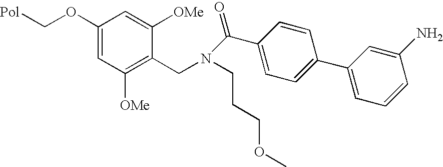

This step is carried out in the same manner as for step 4 of Example 1.

### (4) 3′-(4-Chloro-2,5-dimethyl-benzenesulfonylamino)-biphenyl-4-carboxylic acid (4-ethoxy-2,6-dimethoxy-benzyl)-(3-methoxy-propyl)-amide (10)

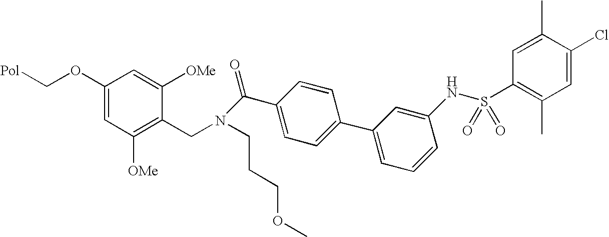

This step is carried out in the same manner as for step 5 of Example 1.

### (5) 3′-(4-Chloro-2,5-dimethyl-benzenesulfonylamino)-biphenyl-4-carboxylic acid (3-methoxy-propyl)-amide

The resin 10 from step 4 (0.12 mmol of bound species) is treated with a 1/4 mixture of TFA and DCM (2 ml) for one hour at room temperature. The resin is drained and washed with DCM (3 times 2 ml). The combined organic phases are then concentrated, taken up in a minimum of methanol and submitted to purification by AP-RP-HPLC (Method A). The product-containing fractions are lyophilized to give the title compound Example 37 as a white powder. HPLC rt=6.33 min (Method D), MS (ESI): 487-489 [M+H]⁺.

### EXAMPLE 38

### 3′-(4-Chloro-2,5-dimethyl-benzenesulfonylamino)-biphenyl-4-carboxylic acid ((S)-1-carbamoyl-2-methyl-propyl)-amide

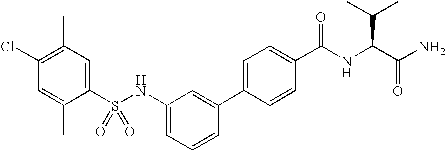

A solution of Example 22 (15 mg, 0.027 mmol) in DMF (180 μl) is treated with triethylamine (19 μl, 0.135 mmol) and HATU (11.5 mg, 0.029 mmol). The resulting solution is stirred at room temperature for five minutes before the addition of a solution of ammonia in MeOH (7M, 50 μl, 0.350 mmol). Stirring is then resumed for one hour before purification by AP-RP-HPLC (Method A). The product-containing fractions are lyophilized to give the title compound Example 38 as a white powder. HPLC rt=4.79 min (Method D), MS (ESI): 536-538 [M+Na]⁺.

### EXAMPLE 39

### 3′-(4-Chloro-2,5-dimethyl-benzenesulfonylamino)-biphenyl-4-carboxylic acid ((S)-2-methyl-1-methylcarbamoyl-propyl)-amide

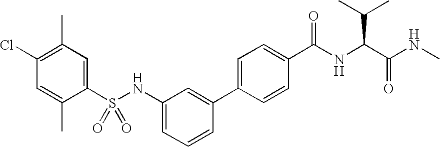

The synthesis is analogous to that of compound Example 38 using methylamine instead of ammonia. HPLC rt=4.98 min (Method B), MS (ESI): 528-530 [M+H]⁺.

1H-NMR (DMSO-d6): δ (ppm) 10.62 (br s, 1H), 8.30 (d, 1H), 7.95 (m, 3H), 7.54 (d, 2H), 7.47 (s, 1H), 7.33 (m, 3H), 7.07 (br m, 1H), 4.24 (m, 1H), 2.61 (d, 3H), 2.54 (s, 3H), 2.36 (s, 3H), 2.11 (m, 1H), 0.91 (m, 6H).

### EXAMPLE 40

### 3′-(4-Chloro-2,5-dimethyl-benzenesulfonylamino)-biphenyl-4-carboxylic acid ((S)-1-dimethylcarbamoyl-2-methyl-propyl)-amide

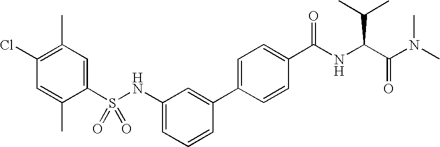

The synthesis is analogous to that of compound Example 38 using dimethylamine instead of ammonia. HPLC rt=5.32 min (Method B), MS (ESI): 542-544 [M+H]⁺.

### Synthesis in Solution

Agents of the invention may also be prepared in solution by a reaction sequence involving Suzuki coupling of boronic acids with corresponding aryl halides, sulfonamidation with appropriate sulfonyl chlorides, ester cleavage and amide coupling, optionally followed by a deprotection step, as shown in reaction scheme 2a below:

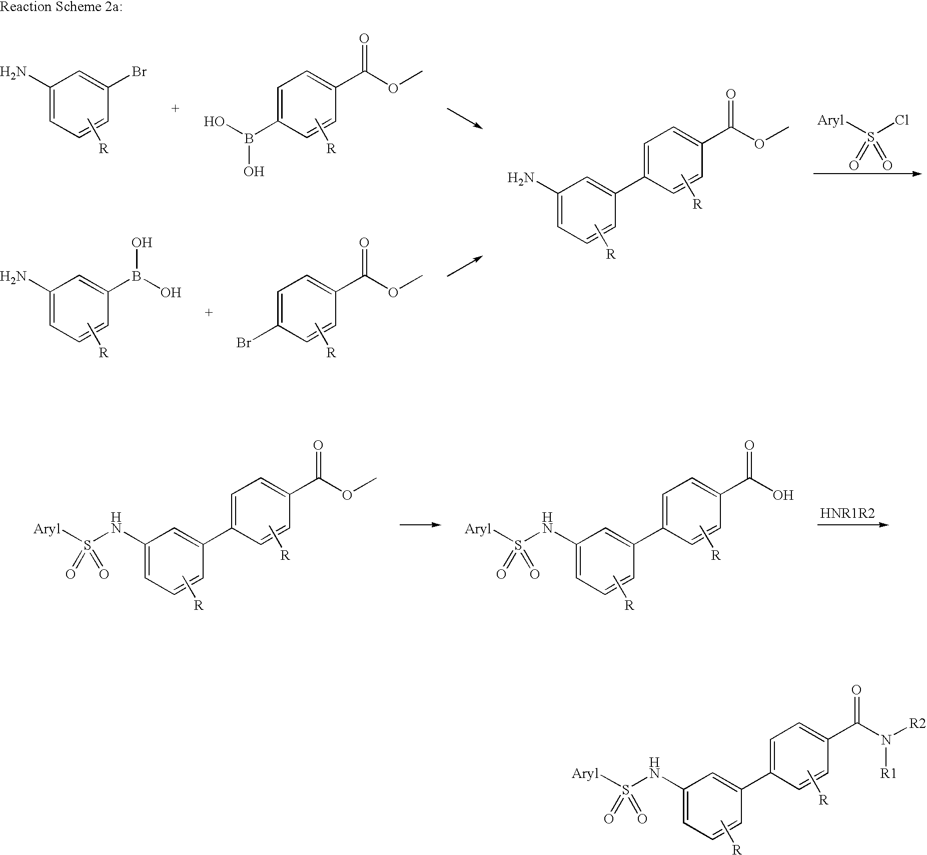

### EXAMPLE 41

### {[3′-(4-Chloro-2,5-dimethyl-benzenesulfonylamino)-2′-methyl-biphenyl-4-carbonyl]-amino}-acetic acid

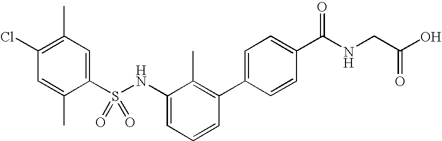

### (1) 3′-Amino-2′-methyl-biphenyl-4-carboxylic acid methyl ester (11)

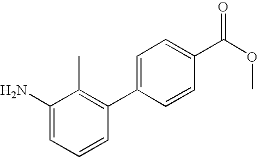

A mixture of 3-Bromo-2-methyl-phenylamine (100 mg, 0.54 mmol), (4-methoxycarbonylphenyl)-boronic acid (106 mg, 0.59 mmol), a 2M aqueous solution of sodium carbonate (1.30 ml, 2.60 mmol) and tetrakis-triphenylphosphinopalladium (31 mg, 0.027 mmol) in DME (2.60 ml) is heated to 150° C. under microwave irradiation for 17 minutes. The reaction mixture is then diluted with EtOAc and filtered over Florisil®. The organic layer is decanted and concentrated to a thick oil which is purified by flash chromatography on silica gel using a gradient of hexane and EtOAc containing 1% of concentrated NH₄OH (from 10% polar solvent to 100% polar solvent). After concentration of the product-containing fractions, the title compound 11 is obtained as a thick oil.

### (2) 3′-(4-Chloro-2,5-dimethyl-benzenesulfonylamino)-2′-methyl-biphenyl-4-carboxylic acid methyl ester (12)

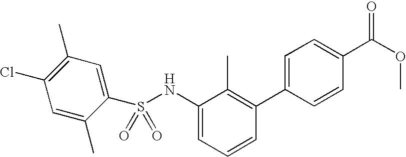

To a solution of 11 (80 mg, 0.33 mmol) in DCE (0.90 ml) at 0° C. is added dropwise a preformed solution of 4-Chloro-2,5-dimethyl-benzenesulfonyl chloride (79 mg, 0.33 mmol) and pyridine (63 μl, 0.65 mmol) in DCE (1.00 ml). The resulting mixture is stirred at 0° C. for 2 hours before dilution with EtOAc (10 ml). The medium is washed three times with 1N aqueous hydrochloric acid solution (10 ml), one time with brine (10 ml), dried over Na₂SO₄, and concentrated to a brown solid. The crude material is purified by flash chromatography on silica gel using a gradient of hexane and EtOAc containing 1% of concentrated NH₄OH (from 10% polar solvent to 100% polar solvent). After concentration of the product-containing fractions, the title compound 12 is obtained as a white powder.

### (3) 3′-(4-Chloro-2,5-dimethyl-benzenesulfonylamino)-2′-methyl-biphenyl-4-carboxylic acid (13)

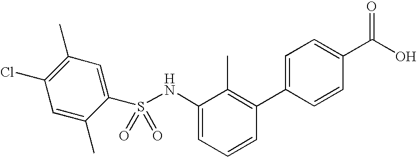

Compound 12 (80.0 mg, 0.18 mmol) is dissolved in a 1/1 mixture of THF and water (1 ml) and treated with lithium hydroxide hydrate (7.5 mg, 0.18 mmol). The resulting mixture is then stirred at room temperature for 16 hours before careful evaporation of methanol under reduced pressure. The resulting aqueous phase is diluted with water (5 ml) and extracted two times with ethyl acetate (5 ml). The aqueous phase is then acidified to pH 1 with 0.1 N aqueous hydrochloric acid solution and extracted three times with EtOAc (5 ml). The combined organic extracts are dried over Na2SO4 and evaporated to yield 13 as a brown powder.

### (4) {[3′-(4-Chloro-2,5-dimethyl-benzenesulfonylamino)-2′-methyl-biphenyl-4-carbonyl]-amino}-acetic acid

The acid 13 (15 mg, 0.035 mmol) and glycine tert-butyl ester (6.9 mg, 0.052 mmol) are dissolved in DMA (300 μl) and treated with HATU (20.0 mg, 0.052 mmol) and DIPEA (18.3 μl, 0.105 mmol). After stirring for 18 hours at rt, the mixture is diluted with methanol and submitted to preparative HPLC purification (Method A). The product-containing fractions are combined, evaporated to dryness and treated with a 1/1 mixture of TFA in DCM for 2 hours at room temperature. The solvents are then removed under reduced pressure, the crude is taken up in tert-butanol and lyophilized to the title compound Example 41, obtained as a white powder. HPLC rt=4.49 min (Method B), MS (ESI): 486-488 [M+H]⁺.

1H-NMR (DMSO-d6): δ (ppm) 12.42 (br s, 1H), 9.77 (br s, 1H), 8.82 (t, 1H), 7.90 (d, 2H), 7.65 (s, 1H), 7.51 (s, 1H), 7.32 (d, 2H), 7.16 (t, 2H), 7.07 (d, 1H), 6.87 (d, 1H), 3,93 (d, 2H), 2.49 (s, 3H), 2.31 (s, 3H), 1.99 (s, 3H).

### EXAMPLE 42

### (S)-2-{[3′-(4-Chloro-2,5-dimethyl-benzenesulfonylamino)-2′-methyl-biphenyl-4-carbonyl]-amino}-3-hydroxy-propionic acid

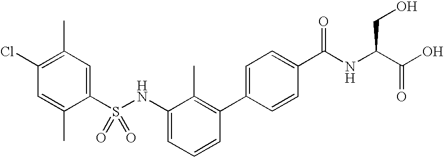

The synthesis of this compound is accomplished analogously to the synthesis of Example 41, using (S)-2-Amino-3-tert-butoxy-propionic acid tert-butyl ester instead of glycine tert-butyl ester in step 4. HPLC rt=5.49 min (Method D), MS (ESI): 517-519 [M+H]⁺.

1H-NMR (DMSO-d6): δ (ppm) 7.93-7.89 (m, 3H), 7.71 (s, 1H), 7.43 (s, 1H), 7.30 (m, 2H), 7.16 (t, 1H), 7.07 (d, 1H), 6.99 (d, 1H), 4.35 (m, 1H), 3,83 (m, 1H), 3.72 (m, 1H), 2.50 (s, 3H), 2.33 (s, 3H), 2.02 (s, 3H).

### EXAMPLE 43

### {[3′-(4-Chloro-2,5-dimethyl-benzenesulfonylamino)-2-methyl-biphenyl-4-carbonyl]-amino}-acetic acid

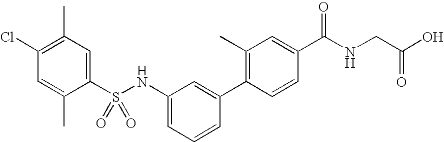

The synthesis of this compound is accomplished analogously to the synthesis of Example 41, using 3-aminophenylboronic acid and 4-Bromo-3-methyl-benzoic acid methyl ester in step 1. HPLC rt=4.43 min (Method B), MS (ESI): 486-488 [M+H]⁺.

1H-NMR (DMSO-d6): δ (ppm) 7.95 (m, 1H), 7.71 (s, 1H), 7.65 (d, 2H), 7.36 (s, 1H), 7.26 (t, 1H), 7.12 (d, 1H), 7.08 (d, 1H), 6.99 (br s, 1H), 6.92 (d, 1H), 3.76 (m, 2H), 2.55 (s, 3H), 2.31 (s, 3H), 2.15 (s, 3H).

### EXAMPLE 44

### (S)-2-{[3′-(4-Chloro-2,5-dimethyl-benzenesulfonylamino)-2-methyl-biphenyl-4-carbonyl]-amino}-3-hydroxy-propionic acid

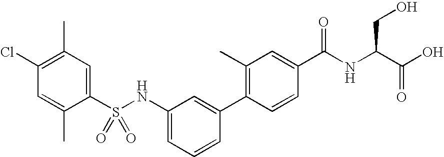

The synthesis of this compound is accomplished analogously to the synthesis of Example 43, using (S)-2-Amino-3-tert-butoxy-propionic acid tert-butyl ester instead of glycine tert-butyl ester. HPLC rt=5.42 min (Method D), MS (ESI): 516-518 [M+H]⁺.

1H-NMR (DMSO-d6): δ (ppm) 10.51 (br s, 1H), 8.34 (d, 1H), 7.95 (m,1H), 7.84-7.71 (m, 3H), 7.47 (s, 1H), 7.31 (t, 1H), 7.14-6.98 (m, 3H), 4.45 (m, 1H), 3.78 (m, 2H), 2.53 (s, 3H), 2.32 (s, 3H), 2.11 (s, 3H).

### EXAMPLE 45

### (S)-2-{[3′-(4-Chloro-2,5-dimethyl-benzenesulfonylamino)-3-methyl-biphenyl-4-carbonyl]-amino}-3-hydroxy-propionic acid

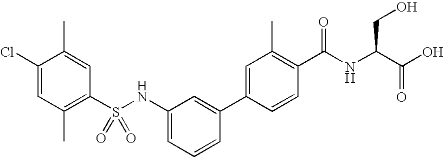

### Beispiel 1: (1) 3′-Amino-3-methyl-biphenyl-4-carboxylic acid methyl ester (14)

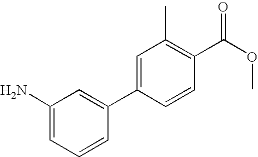

To a mixture of 4-bromo-2-methyl-benzoic acid methyl ester (500 mg, 2.183 mmol) and tetrakis-triphenylphosphinopalladium (126 mg, 0.109 mmol) in DME (12 ml) and aqueous sodium bicarbonate solution (10%, 12.8 ml, 15.28 mmol) is added (3-aminophenyl)-boronic acid monohydrate (338 mg, 2.183 mmol). The mixture is heated to 100° C. for 15 minutes. Another portion of (3-aminophenyl)-boronic acid monohydrate (169 mg, 1.09 mmol) is added and stirring continued for 1 hour. The solvents are then evaporated and the residue is dissolved in EtOAc (50 ml) and washed with saturated sodium bicarbonate solution and brine. The organic layer is dried over sodium sulphate, filtered and evaporated. The crude product is purified by chromatography on silica gel (hexane/EtOAc from 2% to 10%) to give the title compound 14 as a beige powder.

1H-NMR (CDCl3): δ (ppm) 7.97 (d, 1H), 7.44 (s, 1H), 7.43 (d, 1H), 7.25 (t, 1H), 7.03 (d, 1H), 6.95 (br s, 1H), 6.74 (d, 1H), 3.91 (s, 3H), 2.66 (s, 3H).

### (2) 3′-(4-Chloro-2,5-dimethyl-benzenesulfonylamino)-3-methyl-biphenyl-4-carboxylic acid methyl ester (15)

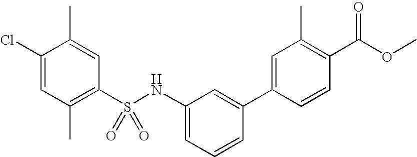

To a solution of the aniline 14 (339 mg, 1.405 mmol) and 4-chloro-2,5-dimethyl-benzenesulfonyl chloride (336 mg, 1.405 mmol) in DCE (14 ml) at 0° C. is added triethylamine (393 μl, 2.81 mmol). The resulting mixture is stirred at 0° C. for 2 hours before dilution with EtOAc (50 ml). The medium is washed twice with 2N—HCl (25 ml), once with brine (25 ml), dried over sodium sulphate and evaporated. The crude product is purified by chromatography on silica gel (hexane/EtOAc from 2% to 10%). After concentration of the product-containing fractions, the title compound 15 is obtained as a white powder.

1H-NMR (CDCl3): δ (ppm) 7.97 (d, 1H), 7.88 (s, 1H), 7.27-7.37 (m, 5H), 7.22 (m, 1H), 7.01 (td, 1H), 3.92 (s, 3H), 2.65 (s, 3H), 2.58 (s, 3H), 2.36 (s, 3H).

### (3) 3′-(4-Chloro-2,5-dimethyl-benzenesulfonylamino)-3-methyl-biphenyl-4-carboxylic acid (16)

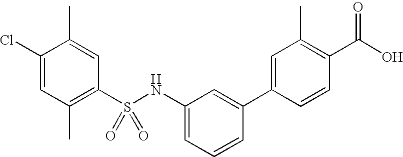

The ester 15 (223 mg, 0.501 mmol) is dissolved in a 1/1/1 mixture of THF, water and ethanol (5 ml) and treated with solid KOH (112 mg, 2.004 mmol). The resulting mixture is then heated under reflux for 2 hours before evaporation of the organic solvents under reduced pressure. The resulting aqueous phase is diluted with water (10 ml) and extracted once with ether (20 ml). The aqueous phase is then acidified to pH 1 with 2N—HCl and extracted three times with EtOAc (20 ml). The combined organic extracts are dried over sodium sulphate and evaporated to yield 16 as a beige powder.

1H-NMR (CDCl3): δ (ppm) 8.12 (d, 1H), 7.99 (s, 1H), 7.22-7.4 (m, 5H), 7.03 (td, 1H), 6.32 (s, 1H), 2.71 (s, 3H), 2.6 (s, 3H), 2.45 (s, 3H), 2.05 (s, 3H).

### (4) (S)-3-tert-Butoxy-2-{[3′-(4-chloro-2,5-dimethyl-benzenesulfonylamino)-3-methyl-biphenyl-4-carbonyl]-amino}-propionic acid tert-butyl ester (17)

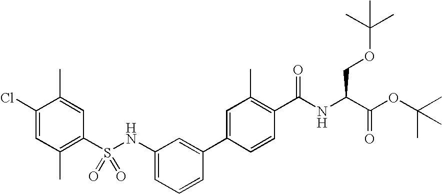

The acid 16 (210 mg, 0.488 mmol), (S)-2-Amino-3-tert-butoxy-propionic acid tert-butyl ester (159 mg, 0.732 mmol) and DIPEA (336 μl, 1.952 mmol) are dissolved in DMF (5 ml) and treated with TBTU (162 mg, 0.488 mmol). After stirring for 2 hours at room temperature, the mixture is evaporated under high vacuum. The crude product is purified by chromatography on silica gel (hexane/EtOAc from 1% to 10%). After concentration of the product-containing fractions, the title compound 17 is obtained as a white powder.

### (5) (S)-3-tert-Butoxy-2-{[3′-(4-chloro-2,5-dimethyl-benzenesulfonylamino)-3-methyl-biphenyl-4-carbonyl]-amino}-propionic acid tert-butyl ester

The ester 17 (170 mg, 0.27 mmol) is dissolved in DCM (3 ml) and treated with TFA (3 ml). After stirring for 2 hours at room temperature the solution is evaporated to dryness. The residue is dissolved in EtOAc (20 ml) and extracted with 2N—NaOH (10 ml). The aqueous layer is then acidified with concentrated HCl and extracted three times with EtOAc (30 ml). The combined organic layers are dried over sodium sulphate and evaporated. The crude product is purified by chromatography on silica gel (hexane/EtOAc from 2% to 100%). After concentration of the product-containing fractions, the title compound Example 45 is obtained as a white powder.

MS (ESI): 515-517 [M−H]⁻, 1H-NMR (DMSO-d6): δ (ppm) 12.6 (br s, 1H), 10.55 (br s, 1H), 8.27 (d, 1H), 7.94 (s, 1H), 7.48 (s, 1H), 7.45 (d, 2H), 7.25-7.35 (m, 5H), 7.05 (m, 1H), 4.44 (m, 1H), 3.77 (d, 2H), 2.54 (s, 3H), 2.42 (s, 3H), 2.35 (s, 3H).

### EXAMPLE 46

### (R)-2-{[3′-(4-Chloro-2,5-dimethyl-benzenesulfonylamino)-3-methyl-biphenyl-4-carbonyl]-amino}-3-hydroxy-propionic acid

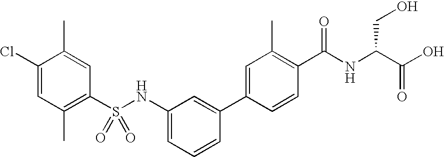

The synthesis of this compound is accomplished analogously to the synthesis of Example 45, using (R)-2-Amino-3-tert-butoxy-propionic acid tert-butyl ester in step 4.

MS (ESI): 515-517 [M−H]⁻, 1H-NMR (DMSO-d6): δ (ppm) 12.6 (br s, 1H), 10.55 (br s, 1H), 8.27 (d, 1H), 7.94 (s, 1H), 7.48 (s, 1H), 7.45 (d, 2H), 7.25-7.35 (m, 5H), 7.05 (m, 1H), 4.44 (m, 1H), 3.77 (d, 2H), 2.54 (s, 3H), 2.42 (s, 3H), 2.35 (s, 3H).

### EXAMPLE 47

### (2S,3R)-2-{[3′-(4-Chloro-2,5-dimethyl-benzenesulfonylamino)-3-methyl-biphenyl-4-carbonyl]-amino}-3-hydroxy-butyric acid

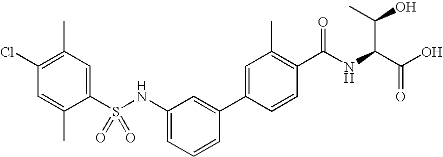

The synthesis of this compound is accomplished analogously to the synthesis of Example 45, using (2S,3R)-2-Amino-3-hydroxy-butyric acid tert-butyl ester hydrochloride in step 4.

MS (ESI): 531-533 [M+H]⁺, 1H-NMR (DMSO-d6): δ (ppm) 12.6 (br s, 1H), 10.56 (s, 1H), 7.94 (m, 2H), 7.44-7.48 (m, 2H), 7.26-7.38 (m, 5H), 7.06 (m, 1H), 4.73 (m, 1H), 4.4 (dd, 1H), 4.19 (m, 1H), 2.54 (s, 3H), 2.42 (s, 3H), 2.35 (s, 3H), 1.18 (d, 3H).

### EXAMPLE 48

### (S)-3-tert-Butoxy-2-{[3′-(4-chloro-2,5-dimethyl-benzenesulfonylamino)-3-methyl-biphenyl-4-carbonyl]-amino}-propionic acid

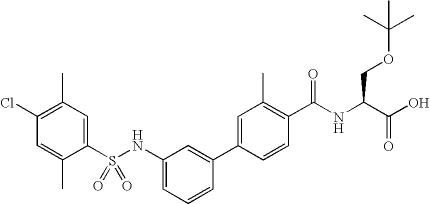

### (1) (S)-3-tert-Butoxy-2-{[3′-(4-chloro-2,5-dimethyl-benzenesulfonylamino)-3-methyl-biphenyl-4-carbonyl]-amino}-propionic acid methyl ester (18)

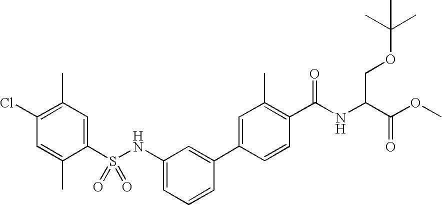

To a stirred mixture of the acid 16 from step 4 of Example 45 (1 g, 2.33 mmol), (S)-2-Amino-3-tert-butoxy-propionic acid methyl ester hydrochloride (739 mg, 3.49 mmol), triethylamine (1.3 ml, 9.3 mmol) and HOBt monohydrate (356 mg, 2.33 mmol) in DCM (20 ml) is added solid EDC hydrochloride (535 mg, 2.79 mmol) and stirring is continued for 16 hours. The mixture is diluted with DCM (50 ml) and washed twice with 2N—HCl (50 ml), water (50 ml), 10% sodium carbonate (50 ml) and brine (20 ml). The organic phase is then dried over sodium sulphate and concentrated to give the title product 18 as white foam which is directly used in the next step.

Optionally, the crude can be further purified by silica gel chromatography using cyclohexane/ethyl acetate from 5% to 50%)

MS (ESI): 587-589 [M+H]⁺

### (2) (S)-3-tert-Butoxy-2-{[3′-(4-chloro-2,5-dimethyl-benzenesulfonylamino)-3-methyl-biphenyl-4-carbonyl]-amino}-propionic acid

The ester 18 from the step above (1.39 g, 2.37 mmol) is dissolved in THF (40 ml) and treated with aqueous 1M-LiOH (9.5 ml, 9.5 mmol). The mixture is stirred vigorously at room temperature for 16 hours. Then most of the THF is evaporated and the residue is diluted with water (50 ml) and washed with ether (100 ml). The aqueous layer is separated and acidified with 2N—HCl and extracted with ether (twice 100 ml). The organic layers are dried over sodium sulphate, filtered and evaporated to furnish the title product Example 48 as white foam.

MS (ESI): 573-575 [M+H]⁺, 1H-NMR (DMSO-d6): δ (ppm) 12.7 (br s, 1H), 10.59 (s, 1H), 8.31 (d, 1H), 7.98 (s, 1H), 7.51 (s, 1H), 7.4 (d, 1H), 7.33 (m, 5H), 7.2 (d, 1H), 4.53 (m, 1H), 3.68 (m, 2H), 2.55 (s, 3H), 2.42 (s, 3H), 2.36 (s, 3H), 1.16 (s, 9H).

### EXAMPLE 49

### 3-{[3′-(4-Chloro-2,5-dimethyl-benzenesulfonylamino)-3-methyl-biphenyl-4-carbonyl]-amino}-azetidine-1,3-dicarboxylic acid 1-tert-butyl ester 3-ethyl ester

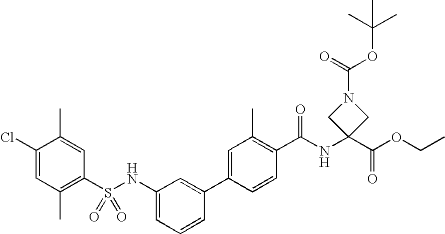

The synthesis of this compound is accomplished analogously to step 1 of the synthesis of PJ@1, using the acid 16 from step 4 of Example 45 and the amino ester 29 (preparation see Example 124).

MS (ESI): 656-658 [M+H]⁺, 1H-NMR (DMSO-d6): δ (ppm) 10.62 (br s, 1H), 9.45 (s, 1H), 7.99 (s, 1H), 7.51 (d, 1H), 7.50 (s, 1H), 7.38 (t, 1H), 7.32 (s, 2H), 7.31 (d, 1H), 7.29 (d, 1H), 7.07 (d, 1H), 4.32-4.20 (br d, 2H), 4.18 (q, 2H), 4.00 (br d, 2H), 2.53 (s, 3H), 2.41 (s, 3H), 2.35 (s, 3H), 1.42 (s, 9H), 1.22 (t, 3H).

### EXAMPLE 50

### 3-{[3′-(4-Chloro-2,5-dimethyl-benzenesulfonylamino)-3-methyl-biphenyl-4-carbonyl]-amino}-azetidine-1,3-dicarboxylic acid mono-tert-butyl ester

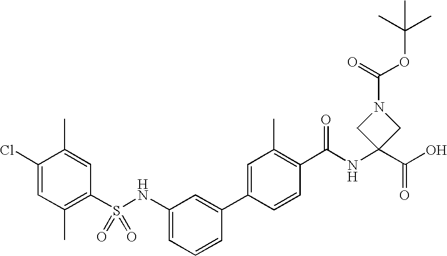

The title compound is obtained by LiOH-hydrolysis of Example 49 as described in step 2 of PJ@1.

MS (ESI): 628-630 [M+H]⁺, 1H-NMR (DMSO-d6): δ (ppm) 13.12 (br s, 1H), 10.62 (br s, 1H), 9.30 (s, 1H), 7.99 (s, 1H), 7.52 (s, 1H), 7.51 (d, 1H), 7.38 (d, 1H), 7.36 (t, 1H), 7.32 (s, 2H), 7.30 (d, 1H), 7.09 (d, 1H), 4.28 (br d, 2H), 4.00 (d, 2H), 2.55 (s, 3H), 2.40 (s, 3H), 2.36 (s, 3H), 1.40 (s, 9H).

### EXAMPLE 51

### 3-{[3′-(4-Chloro-2,5-dimethyl-benzenesulfonylamino)-3-methyl-biphenyl-4-carbonyl]-amino}-azetidine-3-carboxylic acid

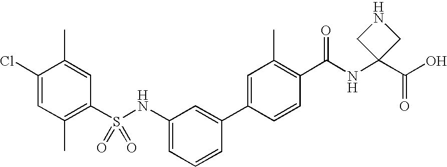

The title compound is obtained as the hydrochloride salt by standard Boc-cleavage of Example 50 with excess of 4M HCl in dioxane at room temperature followed by evaporation.

MS (ESI): 528-530 [M+H]⁺, 1H-NMR (DMSO-d6): δ (ppm) 13.72 (br s, 1H), 10.66 (br s, 1H), 9.70 (br s, 1H), 9.54 (s, 1H), 9.37 (s, 1H), 8.00 (s, 1H), 7.53 (d, 1H), 7.50 (s, 1H), 7.41 (d, 1H), 7.39 (t, 1H), 7.31 (s, 2H), 7.30 (d, 1H), 7.09 (d, 1H), 4.48-4.40 (m, 2H), 4.20-4.11 (m, 2H), 2.56 (s, 3H), 2.45 (s, 3H), 2.37 (s, 3H).

### EXAMPLE 52

### 3-{[3′-(4-Chloro-2,5-dimethyl-benzenesulfonylamino)-3-methyl-biphenyl-4-carbonyl]-amino}-azetidine-3-carboxylic acid methyl ester

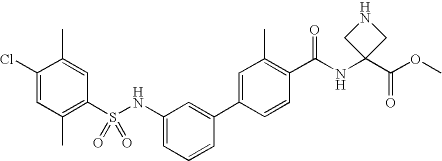

The title compound is obtained by esterification of Example 51 analogously to Example 129.

MS (ESI): 542-544 [M+H]⁺, 1H-NMR (DMSO-d6): δ (ppm) 10.67 (br s, 1H), 9.71 (s, 2H), 9.39 (s, 1H), 7.99 (s, 1H), 7.57 (d, 1H), 7.51 (s, 1H), 7.42 (d, 1H), 7.39 (t, 1H), 7.34 (s, 2H), 7.32 (d, 1H), 7.00 (d, 1H), 4.53-4.44 (m, 2H), 4.20-4.12 (m, 2H), 3.79 (s, 3H), 2.54 (s, 3H), 2.44 (s, 3H), 2.37 (s, 3H).

### EXAMPLE 53

### 3-{[3′-(4-Chloro-2,5-dimethyl-benzenesulfonylamino)-3-methyl-biphenyl-4-carbonyl]-amino}-azetidine-3-carboxylic acid ethyl ester

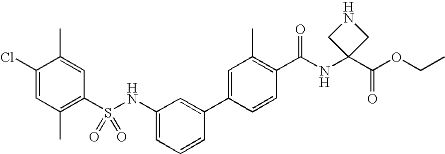

The title compound is obtained by standard Boc-cleavage of Example 49 with excess TFA in DCM at room temperature followed by evaporation.

MS (ESI): 556-558 [M+H]⁺, 1H-NMR (DMSO-d6): δ (ppm) 10.65 (br s, 1H), 9.67 (s, 1H), 9.30 (br s, 2H), 8.00 (s, 1H), 7.55 (d, 1H), 7.51 (s, 1H), 7.42 (d, 1H), 7.39 (t, 1H), 7.35 (s, 2H), 7.31 (d, 1H), 7.08 (td, 1H), 4.50 (d, 2H), 4.21 (q, 2H), 4.18 (d, 2H), 2.53 (s, 3H), 2.46 (s, 3H), 2.35 (s, 3H), 1.23 (t, 3H).

### EXAMPLE 54

### 3-{[3′-(4-Chloro-2,5-dimethyl-benzenesulfonylamino)-3-methyl-biphenyl-4-carbonyl]-amino}-1-methyl-azetidine-3-carboxylic acid ethyl ester

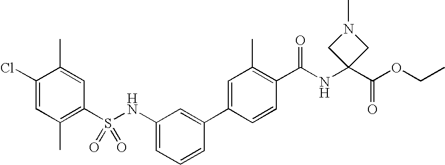

The synthesis of this compound is accomplished by reductive amination of Example 53 with aqueous formaldehyde according to the procedure described in step 3 of Example 162.

MS (ESI): 570-572 [M+H]⁺, 1H-NMR (DMSO-d6): δ (ppm) 10.61 (br s, 1H), 9.32 (s, 1H), 7.99 (s, 1H), 7.50 (s, 1H), 7.43 (d, 1H), 7.38 (d, 1H), 7.35 (t, 1H), 7.32 (s, 2H), 7.30 (d, 1H), 7.08 (d, 1H), 4.13 (q, 2H), 3.61 (d, 2H), 3.36 (d, 2H), 2.55 (s, 3H), 2.40 (s, 3H), 2.37 (s, 3H), 2.28 (s, 3H), 1.21 (t, 3H).

### EXAMPLE 55

### 3-{[3′-(4-Chloro-2,5-dimethyl-benzenesulfonylamino)-3-methyl-biphenyl-4-carbonyl]-amino}-1-methyl-azetidine-3-carboxylic acid

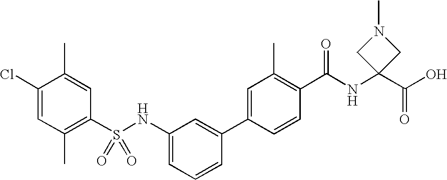

The title compound is obtained by LiOH-hydrolysis of Example 54 analogously to step 2 of PJ@1.

MS (ESI): 542-544 [M+H]⁺, 1H-NMR (DMSO-d6): δ (ppm) 10.62 (br s, 1H), 8.29 (br s, 1H), 8.00 (s, 1H), 7.50 (d, 1H), 7.49 (s, 1H), 7.37 (d, 1H), 7.36 (t, 1H), 7.31 (d, 1H), 7.30 (s, 2H), 7.07 (d, 1H), 4.23 (br d, 2H), 4.11 (d, 2H), 2.79 (br s, 3H), 2.54 (s, 3H), 2.47 (s, 3H), 2.34 (s, 3H).

### EXAMPLE 56

### 1-Acetyl-3-{[3′-(4-chloro-2,5-dimethyl-benzenesulfonylamino)-3-methyl-biphenyl-4-carbonyl]-amino}-azetidine-3-carboxylic acid

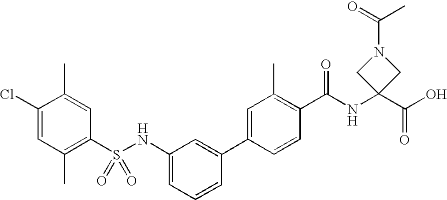

Example 51 (183.3 mg, 0.284 mmol) is dissolved in THF (1 ml). At 0° C. 2N NaOH solution (0.59 ml, 1.20 mmol) is added, followed by acetyl chloride (0.022 ml, 0.31 mmol). The mixture is stirred at RT for 15 hours, diluted with 1 N HCl solution (50 ml) and extracted with EtOAc. Evaporation of the solvents and preparative HPLC (acetonitrile/Water) yields Example 56 as a white powder.

MS (ESI): 570-572 [M+H]⁺, 1H-NMR (DMSO-d6): δ (ppm) 13.18 (br s, 1H), 10.63 (br s, 1H), 9.32 (br s, 1H), 7.99 (s, 1H), 7.50 (s, 1H), 7.49 (d, 1H), 7.38 (d, 1H), 7.35 (t, 1H), 7.33 (s, 2H), 7.30 (d, 1H), 7.08 (d, 1H), 4.60 (d, 1H), 4.23 (d, 1H), 4.18 (d, 1H), 4.00 (d, 1H), 2.53 (s, 3H), 2.42 (s, 3H), 2.38 (s, 3H), 1.80 (s, 3H).

### EXAMPLE 57

### 1-{[3′-(4-Chloro-2,5-dimethyl-benzenesulfonylamino)-3-methyl-biphenyl-4-carbonyl]-amino}-cyclopropanecarboxylic acid

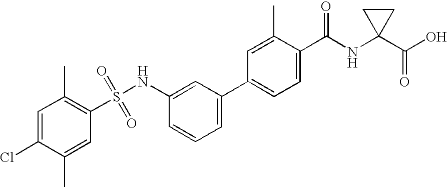

The synthesis of this compound is accomplished analogously to the synthesis of Example 48, using the acid 16 from step 4 of Example 45 and 1-Amino-cyclopropanecarboxylic acid ethyl ester.

MS (ESI): 513-515 [M+H]⁺, 1H-NMR (DMSO-d6): δ (ppm) 12.40 (br s, 1H), 10.57 (br s, 1H), 8.76 (s, 1H), 7.96 (s, 1H), 7.49 (s, 1H), 7.38 (d, 1H), 7.33 (d, 1H), 7.32 (t, 1H), 7.30 (s, 2H), 7.28 (d, 1H), 7.06 (d, 1H), 2.54 (s, 3H), 2.40 (s, 3H), 2.35 (s, 3H), 1.39 (dd, 2H), 1.09 (dd, 2H).

### EXAMPLE 58

### 1-[3′-(4-Chloro-2,5-dimethyl-benzenesulfonylamino)-3-methyl-biphenyl-4-carbonyl]-azetidine-3-carboxylic acid

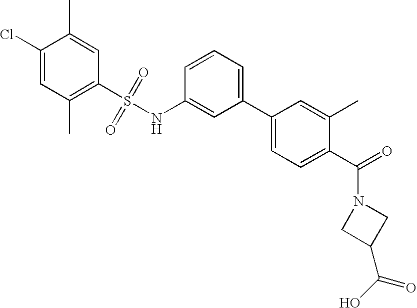

The synthesis of this compound is accomplished analogously to the synthesis of Example 48, using the acid 16 from step 4 of Example 45 and azetidine-3-carboxylic acid methyl ester.

MS (ESI): 513-515 [M+H]⁺, 1H-NMR (DMSO-d6): δ (ppm) 12.69 (br s, 1H), 10.55 (br s, 1H), 7.94 (s, 1H), 7.47 (s, 1H), 7.48-7.25 (m, 6H), 7.05 (d, 1H), 4.23 (t, 1H), 4.10 (t, 1H), 4.06 (dd, 1H), 3.95 (dd, 1H), 3.48-3.35 (m, 1H), 2.53 (s, 3H), 2.36 (s, 3H), 2.35 (s, 3H).

### EXAMPLE 59

### (2S,3S)-2-{[3′-(4-Chloro-2,5-dimethyl-benzenesulfonylamino)-3-methyl-biphenyl-4-carbonyl]-amino}-3-hydroxy-butyric acid

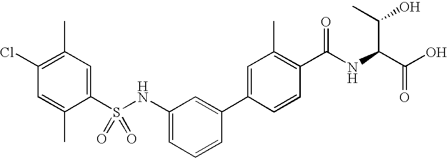

The synthesis of this compound is accomplished analogously to the synthesis of Example 48, using the acid 16 from step 4 of Example 45 and (2S,3S)-2-Amino-3-hydroxy-butyric acid methyl ester hydrochloride.

MS (ESI): 531-533 [M+H]⁺, 1H-NMR (DMSO-d6): δ (ppm) 12.5 (br s, 1H), 10.55 (s, 1H), 8.3 (d, 1H), 7.94 (s, 1H), 7.48 (s, 1H), 7.39 (d, 1H), 7.3 (m, 5H) 7.05 (m, 1H), 4.94 (br m, 1H), 4.36 (dd, 1H), 4.01 (m, 1H), 2.54 (s, 3H), 2.4 (s, 3H), 2.35 (s, 3H), 1.18 (d, 3H).

### EXAMPLE 60

### (S)-2-{[3′-(4-Chloro-2,5-dimethyl-benzenesulfonylamino)-3-methyl-biphenyl-4-carbonyl]-amino}-3-methoxy-propionic acid

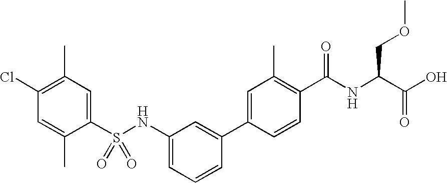

The synthesis of this compound is accomplished analogously to the synthesis of Example 48, using the acid 16 from step 4 of Example 45 and (S)-2-Amino-3-methoxy-propionic acid methyl ester hydrochloride.

MS (ESI): 531-533 [M+H]⁺, 1H-NMR (DMSO-d6): δ (ppm) 12.8 (br s, 1H), 10.58 (s, 1H), 8.52 (d, 1H), 7.96 (s, 1H), 7.49 (s, 1H), 7.41 (d, 1H), 7.31 (m, 5H) 7.07 (m, 1H), 4.6 (br m, 1H), 3.69 (m, 2H), 3.29 (s, 3H), 2.54 (s, 3H), 2.4 (s, 3H), 2.35 (s, 3H).

### EXAMPLE 61

### (S)-2-{[3′-(4-Chloro-2,5-dimethyl-benzenesulfonylamino)-3-methyl-biphenyl-4-carbonyl]-amino}-3-hydroxy-3-methyl-butyric acid

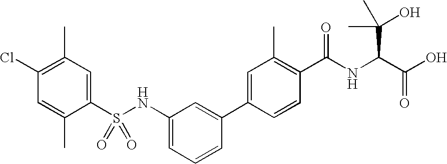

The synthesis of this compound is accomplished analogously to the synthesis of Example 48, using the acid 16 from step 4 of Example 45 and (S)-2-Amino-3-hydroxy-3-methyl-butyric acid methyl ester hydrochloride.

MS (ESI): 543-545 [M−H]⁻, 1H-NMR (DMSO-d6): δ (ppm) 10.4 (v br s, 1H), 7.97 (s, 1H), 7.51 (br s, 1H), 7.49 (s, 1H), 7.45 (d, 1H), 7.35 (m, 5H), 7.08 (d, 1H), 4.16 (m, 1H), 2.55 (s, 3H), 2.43 (s, 3H), 2.36 (s, 3H), 1.17 (s, 3H), 1.08 (s, 3H).

### EXAMPLE 62

### (S)-2-{[3′-(4-Chloro-2,5-dimethyl-benzenesulfonylamino)-3-methyl-biphenyl-4-carbonyl]-amino}-butyric acid

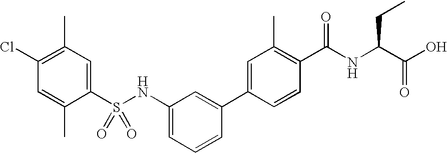

The synthesis of this compound is accomplished analogously to the synthesis of Example 48, using the acid 16 from step 4 of Example 45 and (S)-2-Amino-butyric acid ethyl ester hydrochloride.

MS (ESI): 513-515 [M−H]⁻, 1H-NMR (DMSO-d6): δ (ppm) 12.5 (v br s, 1H), 10.58 (br s, 1H), 8.49 (d, 1H), 7.96 (s, 1H), 7.49 (s, 1H), 7.4 (d, 1H), 7.31 (m, 5H), 7.07 (m, 1H), 4.27 (m, 1H), 2.54 (s, 3H), 2.4 (s, 3H), 2.35 (s, 3H), 1.83 (m, 1H), 1.7 (m, 1H), 0,97 (t, 3H).

### EXAMPLE 63

### (S)-2-{[3′-(4-Chloro-2,5-dimethyl-benzenesulfonylamino)-3-methyl-biphenyl-4-carbonyl]-amino}-propionic acid

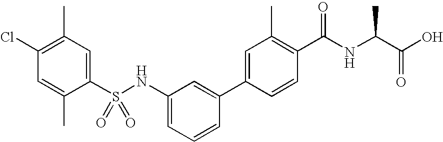

The synthesis of this compound is accomplished analogously to the synthesis of Example 48, using the acid 16 from step 4 of Example 45 and (S)-2-Amino-propionic acid ethyl ester hydrochloride.

MS (ESI): 499-501 [M−H]⁻, 1H-NMR (DMSO-d6): δ (ppm) 12.5 (v br s, 1H), 10.6 (v br s, 1H), 8.43 (br d, 1H), 7.96 (s, 1H), 7.48 (s, 1H), 7.41 (d, 1H), 7.31 (m, 5H), 7.06 (m, 1H), 4.33 (m, 1H), 2.54 (s, 3H), 2.4 (s, 3H), 2.35 (s, 3H), 1.35 (d, 3H).

### EXAMPLE 64

### {[3′-(4-Chloro-2,5-dimethyl-benzenesulfonylamino)-3-methyl-biphenyl-4-carbonyl]-amino}-acetic acid

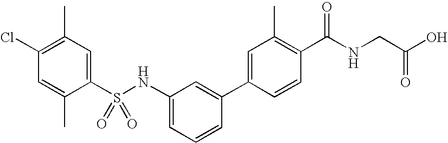

The synthesis of this compound is accomplished analogously to the synthesis of Example 48, using the acid 16 from step 4 of Example 45 and Amino-acetic acid ethyl ester hydrochloride.

MS (ESI): 485-487 [M−H]⁻, 1H-NMR (DMSO-d6): δ (ppm) 12.59 (br s, 1H), 10.59 (br s, 1H), 8.59 (t, 1H), 7.97 (s, 1H), 7.5 (s, 1H), 7.43 (d, 1H), 7.32 (m, 5H), 7.07 (m, 1H), 3.9 (d, 2H), 2.54 (s, 3H), 2.42 (s, 3H), 2.35 (s, 3H).

### EXAMPLE 65

### 3′-(4-Chloro-2,5-dimethyl-benzenesulfonylamino)-3-methyl-biphenyl-4-carboxylic acid cyanomethyl-amide

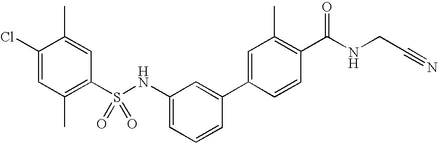

The synthesis of this compound is accomplished analogously to the synthesis of Example 87, using the acid 16 and amino-acetonitrile.

MS (ESI): 466-468 [M−H]⁻, 1H-NMR (DMSO-d6): δ (ppm) 10.6 (v br s, 1H), 8.99 (t, 1H), 7.93 (s, 1H), 7.41 (d, 2H), 7.34 (d, 2H), 7.23 (m, 3H), 6.99 (m, 1H), 4.29 (d, 2H), 2.55 (s, 3H), 2.43 (s, 3H), 2.35 (s, 3H).

### EXAMPLE 66

### 3′-(4-Chloro-2,5-dimethyl-benzenesulfonylamino)-3-methyl-biphenyl-4-carboxylic acid (1H-tetrazol-5-ylmethyl)-amide

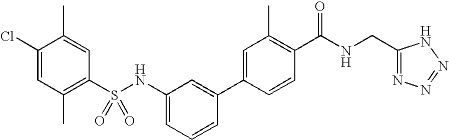

The synthesis of this compound is accomplished analogously to the synthesis of Example 88, using the nitrile Example PJ#10 from above.

MS (ESI): 509-511 [M−H]⁻, 1H-NMR (DMSO-d6): δ (ppm) 16.3 (v br s, 1H), 10.55 (br s, 1H), 8.99 (t, 1H), 7.95 (s, 1H), 7.5 (d, 1H), 7.48 (s, 1H), 7.3 (m, 5H), 7.07 (m, 1H), 4.74 (d, 2H), 2.55 (s, 3H), 2.41 (s, 3H), 2.35 (s, 3H).

### EXAMPLE 67

### 3′-(4-Chloro-2,5-dimethyl-benzenesulfonylamino)-3-methyl-biphenyl-4-carboxylic acid (2-hydroxy-2-methyl-propyl)-amide

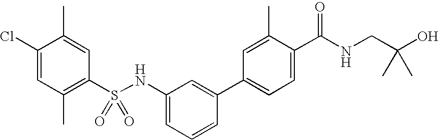

The synthesis of this compound is accomplished analogously to the synthesis of Example 87, using the acid 16 and 1-amino-2-methyl-propan-2-ol.

MS (ESI): 499-501 [M−H]⁻, 1H-NMR (DMSO-d6): δ (ppm) 10.56 (v br s, 1H), 8.07 (t, 1H), 7.93 (s, 1H), 7.46 (s, 1H), 7.39 (d, 1H), 7.28 (m, 5H), 7.03 (m, 1H), 4.47 (s, 1H), 3.21 (d, 2H), 2.54 (s, 3H), 2.4 (s, 3H), 2.35 (s, 3H), 1.14 (s, 6H).

### EXAMPLE 68

### {[5′-(4-Chloro-2,5-dimethyl-benzenesulfonylamino)-2′-methyl-biphenyl-4-carbonyl]-amino}-acetic acid

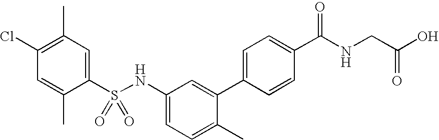

The synthesis of this compound is accomplished analogously to the synthesis of Example 41, using 3-Bromo-4-methyl-phenylamine instead of 3-Bromo-2-methyl-phenylamine in step 1. HPLC rt=4.55 min (Method B), MS (ESI): 486-488 [M+H]⁺.

1H-NMR (DMSO-d6): δ (ppm) 10.42 (br s, 1H), 8.46 (br s, 1H), 7.90 (m, 3H), 7.49 (s, 1H), 7.29 (m, 2H), 7.15 (m, 1H), 6.99 (m, 1H), 6.90 (s, 1H), 3.76 (m, 2H), 2.52 (s, 3H), 2.36 (s, 3H), 2.12 (s, 3H).

### EXAMPLE 69

### (S)-2-{[5′-(4-Chloro-2,5-dimethyl-benzenesulfonylamino)-2′-methyl-biphenyl-4-carbonyl]-amino}-3-hydroxy-propionic acid

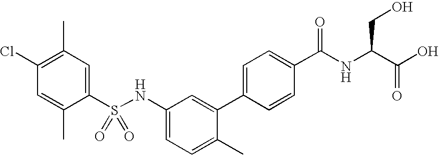

The synthesis of this compound is accomplished analogously to the synthesis of Example 68, using (S)-2-Amino-3-tert-butoxy-propionic acid tert-butyl ester instead of glycine tert-butyl ester. HPLC rt=5.46 min (Method D), MS (ESI): 516-518 [M+H]⁺.

1H-NMR (DMSO-d6): δ (ppm) 12.65 (br s, 1H), 10.40 (br s, 1H), 8.44 (d, 1H), 7.92 (m, 2H), 7.84 (s, 1H), 7.47 (s, 1H), 7.27 (m, 2H), 7.15 (d, 1H), 6.98 (m, 1H), 6.89 (d, 1H), 4.49 (m, 1H), 3.81 (m, 2H), 2.52 (s, 3H), 2.34 (s, 3H), 2.10 (s, 3H).

### EXAMPLE 70

### {[3′-(4-Chloro-2,5-dimethyl-benzenesulfonylamino)-3-methoxy-biphenyl-4-carbonyl]-amino}-acetic acid

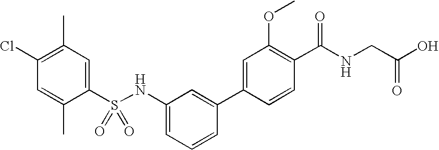

The synthesis of this compound is accomplished analogously to the synthesis of Example 41, using 3-aminophenylboronic acid and 4-Bromo-3-methoxy-benzoic acid methyl ester in step 1. HPLC rt=4.52 min (Method B), MS (ESI): 502-504 [M+H]⁺.

1H-NMR (DMSO-d6): δ (ppm) 8.55 (m, 1H), 7.93 (m, 2H), 7.46 (s, 1H), 7.30-7.10 (m, 6H), 3.99 (s, 3H), 3.85 (m, 2H), 2.54 (s, 3H), 2.34 (s, 3H).

### EXAMPLE 71

### (S)-2-{[3′-(4-Chloro-2,5-dimethyl-benzenesulfonylamino)-3-methoxy-biphenyl-4-carbonyl]-amino}-3-hydroxy-propionic acid

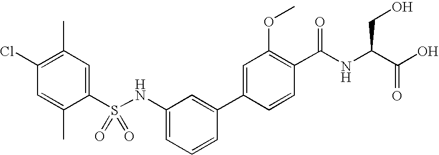

The synthesis of this compound is accomplished analogously to the synthesis of Example 70, using (S)-2-Amino-3-tert-butoxy-propionic acid tert-butyl ester instead of glycine tert-butyl ester. HPLC rt=5.48 min (Method D), MS (ESI): 532-534 [M+H]⁺.

1H-NMR (DMSO-d6): δ (ppm) 12.72 (br s, 1H), 10.57 (br s, 1H), 8.61 (d, 1H), 7.93 (m, 2H), 7.50-7.10 (m, 7H), 4.49 (m, 1H), 4.02 (s, 3H), 3.86 (m, 1H), 3.76 (m, 1H), 2.55 (s, 3H), 2.34 (s, 3H).

### EXAMPLE 72

### (S)-2-({5-[3-(4-Chloro-2,5-dimethyl-benzenesulfonylamino)-phenyl]-pyrazine-2-carbonyl}-amino)-3-hydroxy-propionic acid

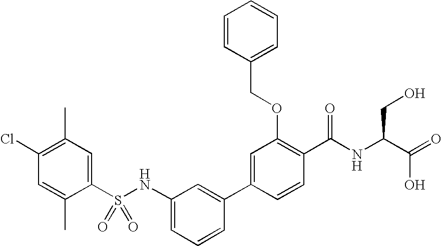

### (1) (S)-2-(4-Bromo-2-hydroxy-benzoylamino)-3-tert-butoxy-propionic acid tert-butyl ester (19)

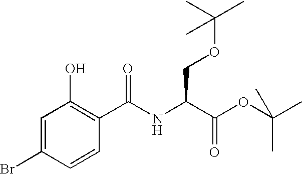

4-Bromo-2-hydroxybenzoic acid methyl ester (1000 mg, 4.61 mmol) and HATU (2100 mg, 5.53 mmol) are dissolved in DMF (10 ml) and treated with DIPEA (2.90 ml, 16.6 mmol). After five minutes of stirring at room temperature, (S)-2-Amino-3-tert-butoxy-propionic acid tert-butyl ester (1450 mg, 5.53 mmol) is added and stirring is resumed for 6 hours. The medium is then concentrated to a thick syrup. This is taken up in EtOAc (75 ml) and washed successively with 1M aqueous HCl solution (2×75 ml) and pH 7 aqueous phosphate buffer (75 ml). The organic phase is dried over Na2SO4 and the crude product is purified by chromatography on silica gel using a 10% to 30% gradient of EtOAc in hexane to furnish 19 as a white powder.

### (2) (S)-2-(2-Benzyloxy-4-bromo-benzoylamino)-3-tert-butoxy-propionic acid tert-butyl ester (20)

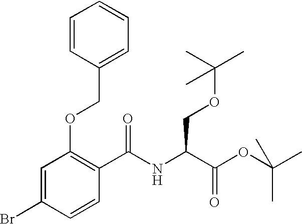

Cesium carbonate (376 mg, 1.14 mmol) and 19 (200 mg, 0.48 mmol) are dissolved together in DMF (2.0 ml) and treated with benzyl bromide (103 μl, 0.86 mmol). The resulting medium is stirred at 60° C. for 16 hours before cooling, dilution with 1M aqueous sodium hydroxide solution (5 ml) and extraction with EtOAc (3×5 ml). The combined organic layers are washed with brine (10 ml), dried over Na2SO4 and concentrated to furnish the title product 20.

### (3) (S)-2-[(3′-Amino-3-benzyloxy-biphenyl-4-carbonyl)-amino]-3-tert-butoxy-propionic acid tert-butyl ester (21)

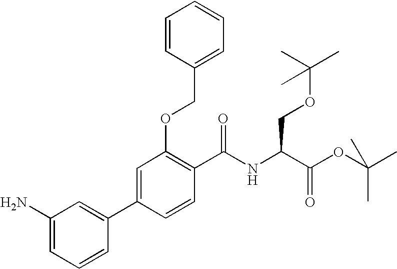

This compound is synthesised in a manner analogous to that used for the synthesis of 14, using 20 instead of 4-bromo-2-methyl-benzoic acid methyl ester.

### (4) (S)-2-{[3-Benzyloxy-3′-(4-chloro-2,5-dimethyl-benzenesulfonylamino)-biphenyl-4-carbonyl]-amino}-3-tert-butoxy-propionic acid tert-butyl ester (22)

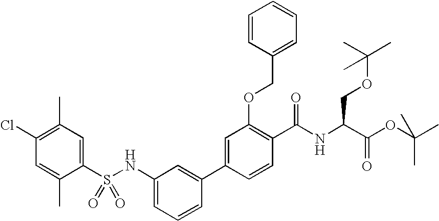

This compound is synthesised in a manner analogous to that used for the synthesis of 15, using 21 instead of 14.

### (5) (S)-2-{5-[3-(4-Chloro-2,5-dimethyl-benzenesulfonylamino)-phenyl]-pyrazine-2-carbonyl}-amino)-3-hydroxy-propionic acid

The intermediate 22 is treated with TFA for one hour at room temperature. TFA is then evaporated under reduced pressure, the residue is taken up in a mixture of DMA, methanol and water and purification is carried out by preparative reverse-phase HPLC (Method A). The product-containing fractions are then lyophilized to give the title compound Example 72 as a white powder. HPLC rt=4.84 min (Method B), MS (ESI): 609-611 [M+H]⁺.

1H-NMR (DMSO-d6): δ (ppm) 10.63 (s, 1H), 8.01 (d, 1H), 7.95 (s, 1H), 7.59 (d, 2H), 7.50 (s, 1H), 7.41-7.30 (m, 7H), 7.18-7.08 (m, 2H), 5.45 (m, 2H), 4.53 (m, 1H), 3.78-3.68 (m, 2H), 2.60 (s, 3H), 2.38 (s, 3H).

### EXAMPLE 73

### (S)-2-{[3′-(4-Chloro-2,5-dimethyl-benzenesulfonylamino)-3-isobutoxy-biphenyl-4-carbonyl]-amino}-3-hydroxy-propionic acid

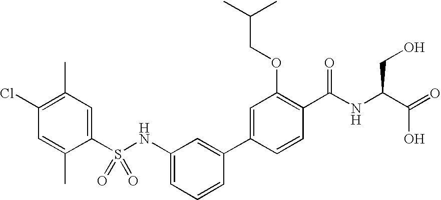

The synthesis of this compound is accomplished analogously to the synthesis of Example 72, using isobutyl bromide instead of benzyl bromide in step 2. HPLC rt=4.87 min (Method B), MS (ESI): 575-577 [M+H]⁺.

1H-NMR (DMSO-d6): δ (ppm) 10.61 (s, 1H), 8.06 (d, 1H), 7.95 (s, 1H), 7.49 (s, 1H), 7.42-7.33 (m, 3H), 7.25 (br s, 1H), 7.16-7.10 (m, 2H), 4.56 (m, 1H), 4.05 (m, 2H), 3.87 (m, 2H), 3.74 (m, 2H), 2.54 (s, 3H), 2.33 (s, 3H), 2.21 (m, 1H), 1.04 (d, 6H).

### EXAMPLE 74

### (S)-2-{[3′-(4-Chloro-2,5-dimethyl-benzenesulfonylamino)-3-(2-methoxy-ethoxy)-biphenyl-4-carbonyl]-amino}-3-hydroxy-propionic acid

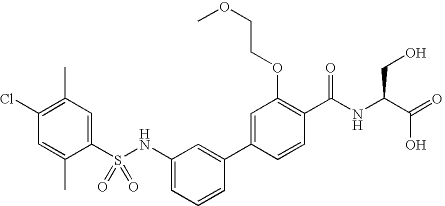

The synthesis of this compound is accomplished analogously to the synthesis of Example 72, using 2-bromoethyl methyl ether instead of benzyl bromide in step 2. HPLC rt=4.46 min (Method B), MS (ESI): 577-579 [M+H]⁺.

1H-NMR (DMSO-d6): δ (ppm) 10.61 (s, 1H), 8.04 (d, 1H), 7.96 (s, 1H), 7.49 (s, 1H), 7.43-7.34 (m, 3H), 7.28 (br s, 1H), 7.16 (m, 1H), 7.10 (m, 1H), 4.53 (m, 1H), 4.37 (m, 2H), 3.87-3.75 (m, 4H), 3.33 (s, 3H), 2.54 (s, 3H), 2.33 (s, 3H).

### EXAMPLE 75

### (S)-2-{[3′-(4-Chloro-2,5-dimethyl-benzenesulfonylamino)-3-propoxy-biphenyl-4-carbonyl]-amino}-3-hydroxy-propionic acid

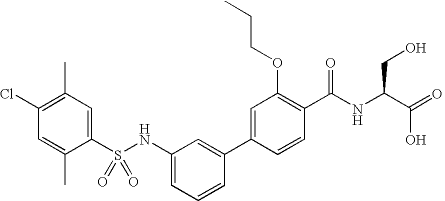

The synthesis of this compound is accomplished analogously to the synthesis of Example 72, using propyl bromide instead of benzyl bromide in step 2. HPLC rt=4.81 min (Method B), MS (ESI): 561-563 [M+H]⁺.

1H-NMR (DMSO-d6): δ (ppm) 10.62 (s, 1H), 8.05 (d, 1H), 7.96 (s, 1H), 7.49 (s, 1H), 7.41-7.33 (m, 3H), 7.23 (br s, 1H), 7.15 (d, 1H), 7.11 (d, 1H), 4.54 (m, 1H), 4.20 (m, 2H), 3.88-3.75 (m, 4H), 2.54 (s, 3H), 2.34 (s, 3H), 1.89 (m, 2H), 1.04 (t, 3H).

### EXAMPLE 76

### (S)-2-{[3′-(4-Chloro-2,5-dimethyl-benzenesulfonylamino)-3-(pyridin-3-ylmethoxy)-biphenyl-4-carbonyl]-amino}-3-hydroxy-propionic acid

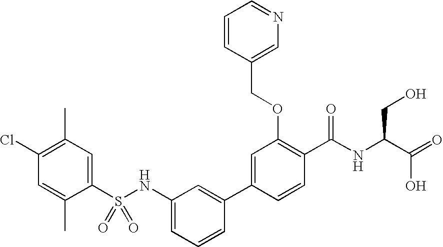

The synthesis of this compound is accomplished analogously to the synthesis of Example 72, using 3-Bromomethyl-pyridine instead of benzyl bromide in step 2. HPLC rt=3.65 min (Method B), MS (ESI): 610-612 [M+H]⁺.

1H-NMR (DMSO-d6): δ (ppm) 10.6 (s, 1H), 8.84 (br s, 1H), 8.74 (d, 1H), 8.50 (d, 1H), 8.20 (d, 1H), 7.97 (m, 2H), 7.58-7.35 (m, 6H), 7.20 (d, 1H), 7.12 (d, 1H), 5.49 (m, 2H), 4.50 (m, 1H), 3.81-3.65 (m, 2H), 2.55 (s, 3H), 2.33 (s, 3H).

### EXAMPLE 77

### {4-[5-(4-Chloro-2,5-dimethyl-benzenesulfonylamino)-pyridin-3-yl]-benzoylamino}-acetic acid

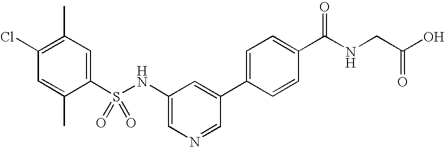

The synthesis of this compound is accomplished analogously to the synthesis of Example 41, using 3-amino-5-bromo-pyridine instead of 3-Bromo-2-methyl-phenylamine in step 1. HPLC rt=3.32 min (Method B), MS (ESI): 473-475 [M+H]⁺.

1H-NMR (DMSO-d6): δ (ppm) 12.57 (br s, 1H), 10.90 (br s, 1H), 8.92 (t, 1H), 8.59 (s, 1H), 8.30 (s, 1H), 7.98 (m, 3H), 7.68 (m, 3H), 7.51 (s, 1H), 3.95 (d, 2H), 2.56 (s, 3H), 2.36 (s, 3H).

### EXAMPLE 78

### (S)-2-{4-[5-(4-Chloro-2,5-dimethyl-benzenesulfonylamino)-pyridin-3-yl]-benzoylamino}-3-hydroxy-propionic acid

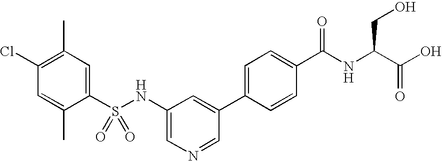

The synthesis of this compound is accomplished analogously to the synthesis of Example 77, using (S)-2-Amino-3-tert-butoxy-propionic acid tert-butyl ester instead of glycine tert-butyl ester. HPLC rt=5.42 min (Method D), MS (ESI): 503-505 [M+H]⁺.

### EXAMPLE 79

### (S)-2-({5-[3-(4-Chloro-2,5-dimethyl-benzenesulfonylamino)-phenyl]-pyrazine-2-carbonyl}-amino)-3-hydroxy-propionic acid

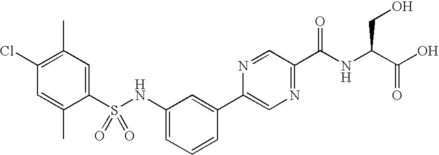

The synthesis of this compound is accomplished analogously to the synthesis of Example 45, using 5-Chloro-pyrazine-2-carboxylic acid methyl ester instead of 4-bromo-2-methyl-benzoic acid methyl ester in step 1. HPLC rt=2.04 min (Method C), MS (ESI): 505-507 [M+H]⁺.

### EXAMPLE 80

### 3′-(4-Chloro-2,5-dimethyl-benzenesulfonylamino)-biphenyl-4-carboxylic acid (2-hydroxy-ethyl)-amide

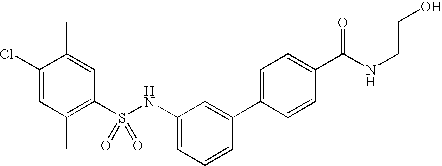

### (1) 3′-(4-Chloro-2,5-dimethyl-benzenesulfonylamino)-biphenyl-4-carboxylic acid methyl ester (23)

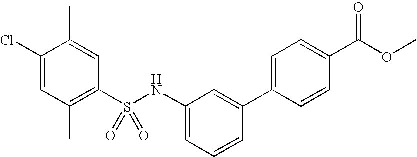

The synthesis of this compound is accomplished analogously to the synthesis of 12, using commercially available 3′-Amino-biphenyl-4-carboxylic acid methyl ester instead of 11.

### (2) 3′-(4-Chloro-2,5-dimethyl-benzenesulfonylamino)-biphenyl-4-carboxylic acid (24)

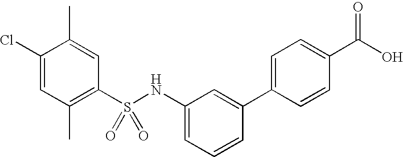

The synthesis of this compound is accomplished analogously to the synthesis of 13.

### (3) 3′-(4-Chloro-2,5-dimethyl-benzenesulfonylamino)-biphenyl-4-carboxylic acid (2-hydroxy-ethyl)-amide

The acid 24 (50.0 mg, 0.119 mmol), HATU (48.5 mg, 0.125 mmol) and Triethylamine (33.3 μl, 0.238 mmol) are stirred together in DMF (0.6 ml) for 5 minutes at room temperature before the addition of 1-amino-2-hydroxyethane (8.0 μl, 0.13 mmol). The resulting solution is then stirred at 120° C. for 5 minutes under microwave irradiation. The mixture is finally diluted with methanol and water and submitted to preparative HPLC purification (Method A). The product-containing fractions are combined, evaporated to dryness, the crude is taken up in tert-butanol and lyophilized to the title compound Example 80, obtained as a white powder. HPLC rt=4.26 min (Method B), MS (ESI): 459-461 [M+H]⁺.

### EXAMPLE 81

### 2-{[3′-(4-Chloro-2,5-dimethyl-benzenesulfonylamino)-biphenyl-4-carbonyl]-amino}-3-hydroxy-propionic acid methyl ester

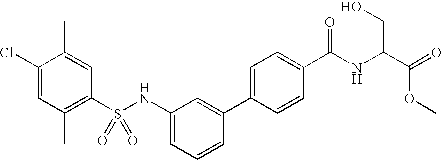

The synthesis of this compound is accomplished analogously to the synthesis of Example 80, using 2-Amino-3-hydroxy-propionic acid methyl ester instead of 1-amino-2-hydroxyethane in step 3. HPLC rt=4.48 min (Method B), MS (ESI): 517-519 [M+H]⁺.

### EXAMPLE 82

### 3′-(4-Chloro-2,5-dimethyl-benzenesulfonylamino)-biphenyl-4-carboxylic acid (2-hydroxy-1-hydroxymethyl-1-methyl-ethyl)-amide

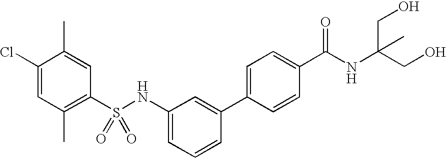

The synthesis of this compound is accomplished analogously to the synthesis of Example 80, using 2-Amino-2-methyl-propane-1,3-diol instead of 1-amino-2-hydroxyethane in step 3. HPLC rt=4.36 min (Method B), MS (ESI): 503-505 [M+H]⁺.

### EXAMPLE 83

### 3′-(4-Chloro-2,5-dimethyl-benzenesulfonylamino)-biphenyl-4-carboxylic acid (2-hydroxy-1-hydroxymethyl-ethyl)-amide

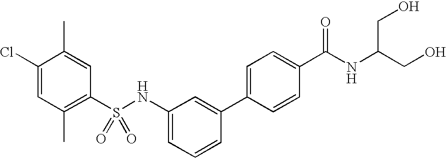

The synthesis of this compound is accomplished analogously to the synthesis of Example 80, using 2-Amino-propane-1,3-diol instead of 1-amino-2-hydroxyethane in step 3. HPLC rt=4.00 min (Method B), MS (ESI): 489-491 [M+H]⁺.

### EXAMPLE 84

### 2-{[3′-(4-Chloro-2,5-dimethyl-benzenesulfonylamino)-biphenyl-4-carbonyl]-amino}-3-hydroxy-2-methyl-propionic acid

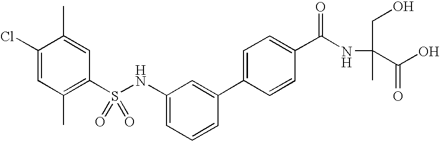

The synthesis of this compound is accomplished analogously to the synthesis of Example 80, using 2-Amino-3-hydroxy-2-methyl-propionic acid instead of 1-amino-2-hydroxyethane in step 3. HPLC rt=4.24 min (Method B), MS (ESI): 517-519 [M+H]⁺.

### EXAMPLE 85

### (S)-2-{[3′-(4-Chloro-2,5-dimethyl-benzenesulfonylamino)-biphenyl-4-carbonyl]-methyl-amino}-3-hydroxy-propionic acid

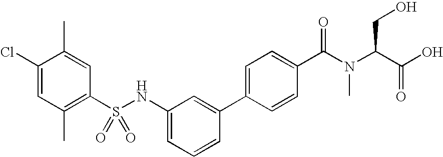

The synthesis of this compound is accomplished analogously to the synthesis of Example 80, using (S)-3-Hydroxy-2-methylamino-propionic acid instead of 1-amino-2-hydroxyethane in step 3. HPLC rt=4.11 min (Method B), MS (ESI): 517-519 [M+H]⁺.

### EXAMPLE 86

### (R)-2-{[3′-(4-Chloro-2,5-dimethyl-benzenesulfonylamino)-biphenyl-4-carbonyl]-methyl-amino}-3-hydroxy-propionic acid

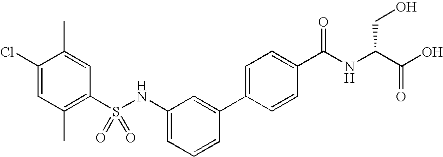

The synthesis of this compound is accomplished analogously to the synthesis of Example 80, using D-serine instead of 1-amino-2-hydroxyethane in step 3. HPLC rt=4.12 min (Method B), MS (ESI): 503-505 [M+H]⁺.

### EXAMPLE 87

### 3′-(4-Chloro-2,5-dimethyl-benzenesulfonylamino)-biphenyl-4-carboxylic acid cyanomethyl-amide

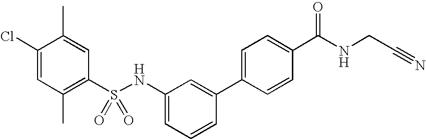

The acid 24 from step 2 of Example 80 (105 mg, 0.252 mmol), DIPEA (129 μl, 0.756 mmol) and amino-acetonitrile (21 mg, 0.378 mmol) are dissolved in DMF (2 ml). TBTU (81 mg, 0.252 mmol) is then added and the mixture is stirred at room temperature for 16 hours. After removal of the solvent under high vacuum, the residue is re-dissolved in ethyl acetate (20 ml) and washed with 2N—HCl, saturated sodium bicarbonate and brine. The organic layer is then dried over sodium sulphate, filtered and evaporated. Purification by chromatography on silica gel (DCM/methanol from 0% to 2%) gives the title compound Example 87 as a white solid.

MS (ESI): 452-454 [M−H]⁻, 1H-NMR (DMSO-d6): δ (ppm) 10.61 (s, 1H), 9.23 (t, 1H), 7.96 (s, 1H), 7.94 (d, 2H), 7.59 (d, 2H), 7.47 (s, 1H), 7.33 (m, 3H), 7.08 (m, 1H), 4.33 (d, 2H), 2.54 (s, 3H), 2.36 (s, 3H).

### EXAMPLE 88

### 3′-(4-Chloro-2,5-dimethyl-benzenesulfonylamino)-biphenyl-4-carboxylic acid (1H-tetrazol-5-ylmethyl)-amide

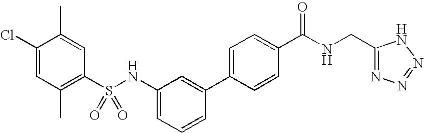

A mixture of Example 87 (45 mg, 0.099 mmol), azidotrimethylsilane (23 mg, 0.198 mmol) and di-n-butyltin oxide (2.5 mg 0.0099 mmol) in DME (1.5 ml) is placed in a microwave vial, sealed and heated under microwave irradiation at 150° C. for 10 minutes. The vial is then opened and another portion of azidotrimethylsilane and di-n-butyltin oxide is added. Heating at 150° C. is repeated for 10 minutes. After cooling the crude mixture is evaporated to dryness, dissolved in 2N—NaOH (10 ml) and washed twice with ether (20 ml). The aqueous layer is acidified with 2N—HCl and extracted three times with DCM. The combined organic layers are dried over sodium sulphate, filtered and evaporated to give the title compound Example 88 as a white powder.

MS (ESI): 495-497 [M−H]⁻, 1H-NMR (DMSO-d6): δ (ppm) 10.6 (brs, 1H), 9.19 (t, 1H), 7.96 (m, 3H), 7.57 (d, 2H), 7.47 (s, 1H), 7.34 (m, 3H), 7.08 (m, 1H), 4.74 (d, 2H), 2.54 (s, 3H), 2.36 (s, 3H).

### EXAMPLE 89

### 3′-(4-Chloro-2,5-dimethyl-benzenesulfonylamino)-biphenyl-4-carboxylic acid (3,3,3-trifluoro-2-hydroxy-propyl)-amide

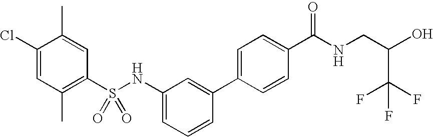

The synthesis of this compound is accomplished analogously to the synthesis of Example 87, using the acid 24 and 3-amino-1,1,1-trifluoro-propan-2-ol.

MS (ESI): 525-527 [M−H]⁻, 1H-NMR (DMSO-d6): δ (ppm) 10.54 (br s, 1H), 8.75 (t, 1H), 7.96 (s, 1H), 7.93 (d, 2H), 7.56 (d, 2H), 7.46 (s, 1H), 7.31 (m, 3H), 7.07 (m, 1H), 6.50 (d, 1H), 4.21 (m, 1H), 3.64 (m, 1H), 3.33 (m, 1H), 2.54 (s, 3H), 2.36 (s, 3H).

### EXAMPLE 90

### 3′-(4-Chloro-2,5-dimethyl-benzenesulfonylamino)-biphenyl-4-carboxylic acid (2-fluoro-ethyl)-amide

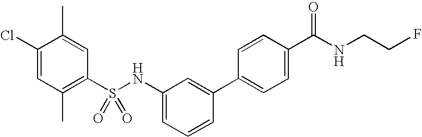

The synthesis of this compound is accomplished analogously to the synthesis of Example 87, using the acid 24 and 3-fluoro-propylamine.

MS (ESI): 459-461 [M−H]⁻, 1H-NMR (DMSO-d6): δ (ppm) 10.6 (br s, 1H), 8.74 (t, 1H), 7.96 (s, 1H), 7.93 (d, 2H), 7.55 (d, 2H), 7.47 (s, 1H), 7.32 (m, 3H), 7.07 (m, 1H), 4.61 (t, 1H), 4.49 (t, 1H), 3.61 (q, 1H), 3.55 (q, 1H), 2.54 (s, 3H), 2.36 (s, 3H).

### EXAMPLE 91

### 3′-(4-Chloro-2,5-dimethyl-benzenesulfonylamino)-biphenyl-4-carboxylic acid (2,2-difluoro-ethyl)-amide

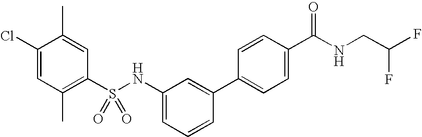

The synthesis of this compound is accomplished analogously to the synthesis of Example 87, using the acid 24 and 3,3-difluoro-propylamine.

MS (ESI): 477-479 [M−H]⁻, 1H-NMR (DMSO-d6): δ (ppm) 10.61 (br s, 1H), 8.89 (t, 1H), 7.96 (s, 1H), 7.94 (d, 2H), 7.57 (d, 2H), 7.47 (s, 1H), 7.33 (m, 3H), 7.08 (m, 1H), 6.12 (tt, 1H), 3.69 (m, 2H), 2.54 (s, 3H), 2.36 (s, 3H).

### EXAMPLE 92

### 3′-(4-Chloro-2,5-dimethyl-benzenesulfonylamino)-biphenyl-4-carboxylic acid (2,2,2-trifluoro-ethyl)-amide

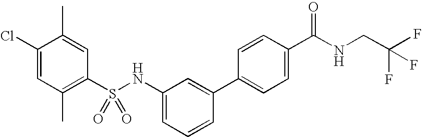

The synthesis of this compound is accomplished analogously to the synthesis of Example 87, using the acid 24 and 3,3,3-trifluoro-propylamine.

MS (ESI): 495-497 [M−H]⁻, 1H-NMR (DMSO-d6): δ (ppm) 10.58 (br m, 1H), 9.11 (br m, 1H), 7.96 (m, 3H), 7.59 (d, 2H), 7.47 (s, 1H), 7.33 (m, 3H), 7.08 (m, 1H), 4.11 (m, 2H), 2.54 (s, 3H), 2.36 (s, 3H).

### EXAMPLE 93

### 3′-(4-Chloro-2,5-dimethyl-benzenesulfonylamino)-biphenyl-4-carboxylic acid (2-hydroxy-2-methyl-propyl)-amide

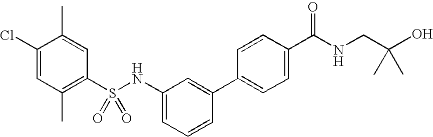

The synthesis of this compound is accomplished analogously to the synthesis of Example 87, using the acid 24 and 1-amino-2-methyl-propan-2-ol.

MS (ESI): 485-487 [M−H]⁻, 1H-NMR (DMSO-d6): δ (ppm) 10.59 (br s, 1H), 8.28 (t, 1H), 7.95 (s, 1H), 7.93 (d, 2H), 7.55 (d, 2H), 7.47 (s, 1H), 7.33 (m, 3H), 7.07 (m, 1H), 4.56 (s, 1H), 3.27 (d, 2H), 2.54 (s, 3H), 2.36 (s, 3H), 1.12 (s, 6H).

### EXAMPLE 94

### 3′-(4-Chloro-2,5-dimethyl-benzenesulfonylamino)-biphenyl-4-carboxylic acid (2-methoxy-1-methyl-ethyl)-amide

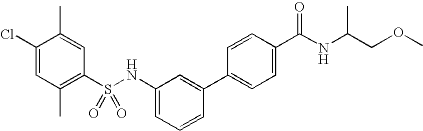

The synthesis of this compound is accomplished analogously to the synthesis of Example 87, using the acid 24 and 2-methoxy-1-methyl-ethylamine.

MS (ESI): 485-487 [M−H]⁻, 1H-NMR (DMSO-d6): δ (ppm) 10.59 (br s, 1H), 8.24 (d, 1H), 7.94 (s, 1H), 7.9 (d, 2H), 7.54 (d, 2H), 7.46 (s, 1H), 7.31 (m, 3H), 7.06 (m, 1H), 4.21 (m, 1H), 3.41 (m, 1H), 3.29 (m, 1H), 3.27 (s, 3H), 2.54 (s, 3H), 2.35 (s, 3H), 1.15 (d, 3H).

### EXAMPLE 95

### 3′-(4-Chloro-2,5-dimethyl-benzenesulfonylamino)-biphenyl-4-carboxylic acid ((S)-2-methoxy-1-methyl-ethyl)-amide

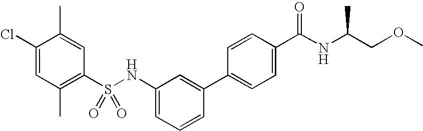

The synthesis of this compound is accomplished analogously to the synthesis of Example 87, using the acid 24 and (S)-2-methoxy-1-methyl-ethylamine.

MS (ESI): 485-487 [M−H]⁻, 1H-NMR (DMSO-d6): δ (ppm) 10.59 (br s, 1H), 8.24 (d, 1H), 7.94 (s, 1H), 7.9 (d, 2H), 7.54 (d, 2H), 7.46 (s, 1H), 7.31 (m, 3H), 7.06 (m, 1H), 4.21 (m, 1H), 3.41 (m, 1H), 3.29 (m, 1H), 3.27 (s, 3H), 2.54 (s, 3H), 2.35 (s, 3H), 1.15 (d, 3H).

### EXAMPLE 96

### 3′-(4-Chloro-2,5-dimethyl-benzenesulfonylamino)-biphenyl-4-carboxylic acid (2-methoxy-ethyl)-amide

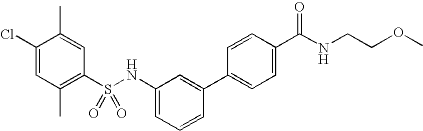

The synthesis of this compound is accomplished analogously to the synthesis of Example 87, using the acid 24 and 2-methoxy-ethylamine.

MS (ESI): 471-473 [M−H]⁻, 1H-NMR (DMSO-d6): δ (ppm) 10.58 (br s, 1H), 8.55 (br s, 1H), 7.95 (s, 1H), 7.91 (d, 2H), 7.54 (d, 2H), 7.47 (s, 1H), 7.33 (m, 3H), 7.06 (m, 1H), 3.46 (m, 4H), 3.27 (s, 3H), 2.54 (s, 3H), 2.36 (s, 3H)

### EXAMPLE 97

### 3′-(4-Chloro-2,5-dimethyl-benzenesulfonylamino)-biphenyl-4-carboxylic acid (2-amino-2-methyl-propyl)-amide

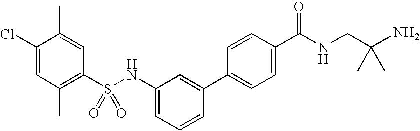

The acid 24 from step 2 of Example 80 (184 mg, 0.442 mmol) is dissolved in DCM (4 ml) and cooled in an ice-bath. To this solution is added DIPEA (150 μl, 0.884 mmol) and isobutyl chloroformate (69 μl, 0.53 mmol) and stirring is continued for 15 minutes. This solution is added drop wise to a cooled solution of 2-methyl-propane-1,2-diamine (390 mg, 4.42 mmol) in DCM (4 ml) and the mixture is stirred for 2 hours at 0° C. Quenching with water (10 ml) and extraction with DCM (twice 20 ml) and ethyl acetate (twice 20 ml) gives after drying and evaporation the title compound Example 97 as off-white powder.

MS (ESI): 484-486 [M−H]⁻, 1H-NMR (DMSO-d6): δ (ppm) 8.44 (br t, 1H), 7.91 (d, 2H), 7.9 (s, 1H), 7.53 (d, 2H), 7.31 (brs, 1H), 7.19 (brs, 1H), 7.14 (m, 1H), 7.04 (m, 1H), 6.89 (m, 1H), 4.2-6.1 (br s, 3H), 3.29 (d, 2H), 2.54 (s, 3H), 2.34 (s, 3H), 1.12 (s, 6H).

### EXAMPLE 98

### 4-[3′-(4-Chloro-2,5-dimethyl-benzenesulfonylamino)-biphenyl-4-carbonyl]-piperazine-2-carboxylic acid

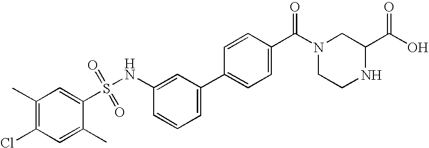

The synthesis of this compound is accomplished analogously to the synthesis of Example 80, using piperazine-1,2-dicarboxylic acid 1-tert-butyl ester 2-methyl ester instead of 1-amino-2-hydroxyethane and EDC as coupling reagent in step 3, followed by TFA mediated BOC-removal and saponification of the methyl ester using 1M LiOH in THF.

MS (ESI): 528-530 [M+H]⁺, 1H-NMR (DMSO-d6): δ (ppm) 10.58 (br s, 1H), 8.64 (br s, 2H, NH₂⁺), 7.85 (s, 1H), 7.53 (d, 2H), 7.47 (d, 2H), 7.40 (s, 1H), 7.34 (s, 1H), 7.33 (t, 1H), 7.31 (d, 1H), 7.10 (d, 1H), 4.04 (brd, 1H), 3.73 (brd, 1H), 3.39 (dd, 1H), 3.29 (m, 1H), 3.23 (m, 1H), 3.05 (dt, 1H), 2.78 (m, 1H), 2.56 (s, 3H), 2.34 (s, 3H).

Alternatively, agents of the invention may also be prepared by a reaction sequence involving an amide coupling between a protected aniline carboxylic acid and an amine, followed by sulfonamidation with appropriate sulfonyl chlorides, optionally followed by a deprotection step, as shown in reaction scheme 2b below:

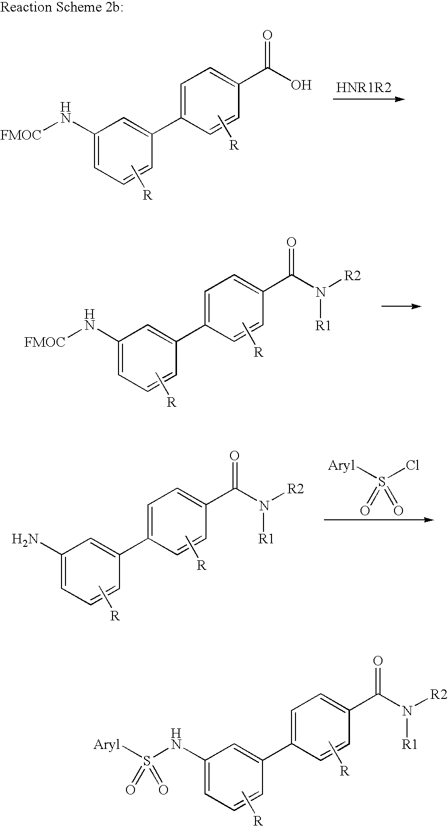

### EXAMPLE 99

### (S)-2-{[3′-(Benzofuran-2-sulfonylamino)-biphenyl-4-carbonyl]-amino}-3-hydroxy-propionic acid

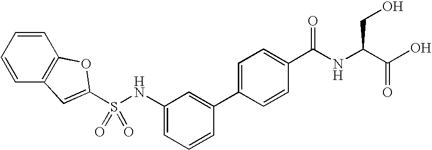

### (1) (S)-3-tert-Butoxy-2-{[3′-(9H-fluoren-9-ylmethoxycarbonylamino)-biphenyl-4-carbonyl]-amino}-propionic acid tert-butyl ester (25)

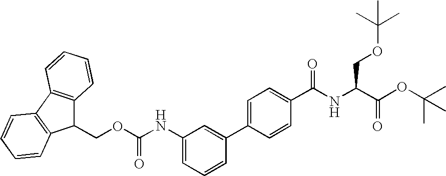

A solution of 3′-(9H-Fluoren-9-ylmethoxycarbonylamino)-biphenyl-4-carboxylic acid (3 g, 6.889 mmol) and (S)-2-Amino-3-tert-butoxy-propionic acid tert-butyl ester (1.647 g, 7.578 mmol) in 50 ml of THF is treated successively with DIPEA (3.55 ml, 20.667 mmol), HOBT (1.024 g, 7.578 mmol) and EDC hydrochloride (1.453 g, 7.578 mmol). The mixture is stirred for 17 hours, diluted with EtOAc (200 ml), washed with 2N—HCl (200 ml), 2N—NaOH (100 ml), water and brine, dried and evaporated. The crude is then purified by chromatography on silica gel (cyclohexane/EtOAc from 5% to 50%). The product containing fractions are evaporated to give the title compound 25 as white solid.

### (2) (S)-2-[(3′-Amino-biphenyl-4-carbonyl)-amino]-3-tert-butoxy-propionic acid tert-butyl ester (26)

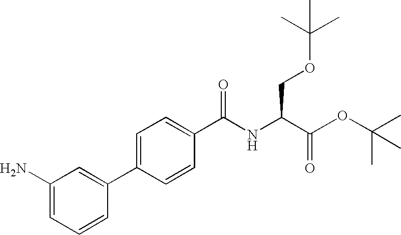

A solution of intermediate 25 (3.7 g, 5.829 mmol) in DCM (50 ml) is treated with tris(2-aminoethyl)amine (43.6 ml, 291.5 mmol) and stirred for 40 minutes. To the stirred cloudy solution is then carefully added brine (60 ml). After the exothermic reaction has settled, the aqueous layer is separated and extracted twice with DCM (50 ml). The combined organic layers are then washed three times with a phosphate buffer (pH 5.6), dried and evaporated. The crude oil is purified by chromatography on silica gel (cyclohexane/EtOAc from 5% to 20%) to give the title compound 26 as white powder.

### (3) (S)-2-{[3′-(Benzofuran-2-sulfonylamino)-biphenyl-4-carbonyl]-amino}-3-hydroxy-propionic acid

The aniline 26 (100 mg, 0.2 mmol) is dissolved in pyridine (1 ml) and treated with a solution of benzofuran-2-sulfonyl chloride (52.5 mg, 0.2 mmol) in DCM (1 ml). After stirring for 16 hours the solution is diluted with EtOAc (20 ml) and washed three times with 2N—HCl (20 ml) and once with saturated sodium bicarbonate (10 ml). It is dried and evaporated. This crude is then dissolved in DCM (1 ml) and TFA (1 ml) and stirred over night. After evaporation the residue is taken up in 2N—NaOH (10 ml) and washed with ether (20 ml). The aqueous layer is then acidified to pH˜3 (upon which a cloudy precipitate is formed) and extracted twice with EtOAc (50 ml). The organic layers are dried and evaporated to give the title compound as beige powder.

MS (ESI): 481 [M+H]⁺, 1H-NMR (DMSO-d6): δ (ppm) 12.6 (br s, 1H), 11.08 (br s, 1H), 8.45 (d, 1H), 7.96 (d, 2H), 7.77 (d, 1H), 7.72 (, 2H), 7.61 (d, 2H), 7.53 (m, 1H), 7.35-7.49 (m. 4H), 7.22 (m, 1H), 5.0 (br s, 1H), 4.51 (m, 1H), 3.82 (m, 2H).

### EXAMPLE 100

### (S)-2-{[3′-(Benzo[b]thiophene-3-sulfonylamino)-biphenyl-4-carbonyl]-amino}-3-hydroxy-propionic acid

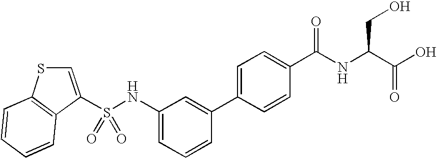

The synthesis of this compound is accomplished analogously to the synthesis of Example 99, using the intermediate 26 and benzo[b]thiophene-3-sulfonyl chloride.

MS (ESI): 497 [M+H]⁺, 1H-NMR (DMSO-d6): δ (ppm) 10.7 (br s, 1H), 8.68 (d, 1H), 8.31 (m, 1H), 8.26 (d, 1H), 8.09 (d, 1H), 7.93 (d, 2H), 7.45-7.58 (m, 4H), 7.27-7.38 (m, 4H), 7.11 (m, 1H), 4.36 (m, 1H), 3.76 (m, 2H).

### EXAMPLE 101

### (S)-3-Hydroxy-2-{[3′-(thiophene-2-sulfonylamino)-biphenyl-4-carbonyl]-amino}-propionic acid

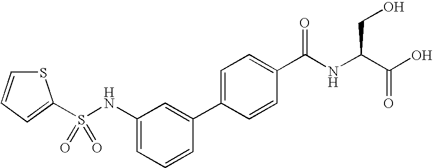

The synthesis of this compound is accomplished analogously to the synthesis of Example 99, using the intermediate 26 and thiophene-2-sulfonyl chloride.

MS (ESI): 447 [M+H]⁺, 1H-NMR (DMSO-d6): δ (ppm) 12.6 (br s, 1H), 10.55 (br s, 1H), 8.45 (d, 1H), 7.99 (d, 2H), 7.9 (dd, 1H), 7.65 (d, 2H), 7.59 (dd, 1H), 7.46 (m, 1H), 7.37-7.47 (m, 3H), 7.18 (m, 1H), 7.14 (m, 1H), 4.51 (m, 1H), 3.82 (m, 2H).

### EXAMPLE 102

### (S)-2-{[3′-(2,4-Dimethyl-thiazole-5-sulfonylamino)-biphenyl-4-carbonyl]-amino}-3-hydroxy-propionic acid

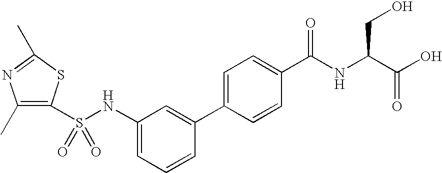

The synthesis of this compound is accomplished analogously to the synthesis of Example 99, using the intermediate 26 and 2,4-dimethyl-thiazole-5-sulfonyl chloride.

MS (ESI): 476 [M+H]⁺, 1H-NMR (DMSO-d6): δ (ppm) 10.7 (br s, 1H), 8.44 (br d, 1H), 7.99 (d, 2H), 7.66 (d, 2H), 7.38-7.52 (m, 3H), 7.17 (brd, 1H), 4.50 (m, 1H), 3.82 (m, 2H), 2.6 (s, 3H), 2.42 (s, 3H).

### EXAMPLE 103

### (S)-2-{[3′-(5-Chloro-1,3-dimethyl-1H-pyrazole-4-sulfonylamino)-biphenyl-4-carbonyl]-amino}-3-hydroxy-propionic acid

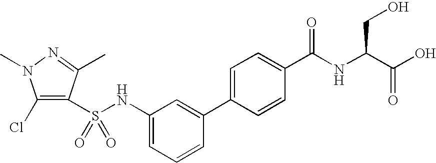

The synthesis of this compound is accomplished analogously to the synthesis of Example 99, using the intermediate 26 and 5-chloro-1,3-dimethyl-1H-pyrazole-4-sulfonyl chloride.

MS (ESI): 491-493 [M−H]⁻, 1H-NMR (DMSO-d6): δ (ppm) 10.57 (br s, 1H), 8.45 (d, 1H), 7.99 (d, 2H), 7.65 (d, 2H), 7.36-7.44 (m, 3H), 7.12 (dt, 1H), 4.51 (m, 1H), 3.83 (m, 2H), 3.73 (s, 3H), 2.26 (s, 3H).

### EXAMPLE 104

### (S)-2-{[3′-(1,2-Dimethyl-1H-imidazole-4-sulfonylamino)-biphenyl-4-carbonyl]-amino}-3-hydroxy-propionic acid

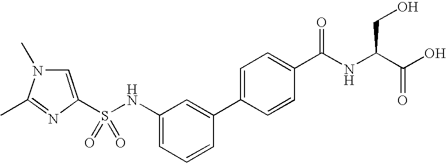

The synthesis of this compound is accomplished analogously to the synthesis of Example 99, using the intermediate 26 and 1,2-dimethyl-1H-imidazole-4-sulfonyl chloride.

MS (ESI): 459 [M+H]⁺, 1H-NMR (DMSO-d6): δ (ppm) 12.1 (br s, 1H), 10.26 (d, 1H), 8.43 (t, 1H), 7.99 (d, 2H), 7.81 (d, 1H), 7.72 (t, 1H), 7.66 (d, 2H), 7.52 (s, 1H), 7.34 (d, 1H), 7.17 (m, 1H), 4.99 (br s, 1H), 4.52 (m, 1H), 3.83 (m, 2H), 2.29 (s, 3H), 1.93 (s, 3H).

### EXAMPLE 105

### (S)-3-Hydroxy-2-{[3′-(1,3,5-trimethyl-1H-pyrazole-4-sulfonylamino)-biphenyl-4-carbonyl]-amino}-propionic acid

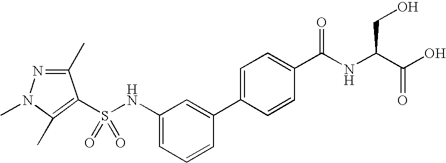

The synthesis of this compound is accomplished analogously to the synthesis of Example 99, using the intermediate 26 and 1,3,5-trimethyl-1H-pyrazole-4-sulfonyl chloride.

MS (ESI): 473 [M+H]⁺, 1H-NMR (DMSO-d6): δ (ppm) 12.6 (brs, 1H), 10.17 (s, 1H), 8.43 (, 1H), 7.97 (d, 2H), 7.61 (d, 2H), 7.33-7.30 (m, 3H), 7.07 (td, 1H), 4.98 (br s, 1H), 4.49 (m, 1H), 3.81 (m, 2H), 3.62 (s, 3H), 2.34 (s, 3H), 2.19 (s, 3H).

### EXAMPLE 106

### (S)-2-{[3′-(4,5-Dichloro-thiophene-2-sulfonylamino)-biphenyl-4-carbonyl]-amino}-3-hydroxy-propionic acid

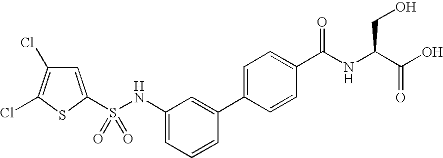

The synthesis of this compound is accomplished analogously to the synthesis of Example 99, using the intermediate 26 and 4,5-dichloro-thiophene-2-sulfonyl chloride.

MS (ESI): 513-515 [M−H]⁻, 1H-NMR (DMSO-d6): δ (ppm) 8.32 (br m, 1H), 7.94 (d, 2H), 7.64 (d, 2H), 7.46 (s, 1H), 7.2-7.36 (m, 3H), 7.07 (m, 1H), 4.42 (m, 1H), 3.78 (m, 3H).

### EXAMPLE 107

### (S)-3-Hydroxy-2-{[3′-(thiophene-3-sulfonylamino)-biphenyl-4-carbonyl]-amino}-propionic acid

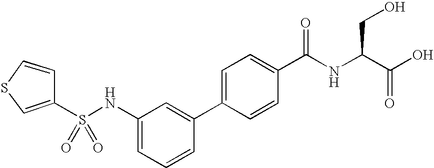

The synthesis of this compound is accomplished analogously to the synthesis of Example 99, using the intermediate 26 and thiophene-3-sulfonyl chloride.

MS (ESI): 447 [M+H]⁺, 1H-NMR (DMSO-d6): δ (ppm) 12.5 (br s, 1H), 10.35 (s, 1H), 8.46 (br m, 1H), 8.21 (br s, 1H), 7.98 (d, 2H), 7.7 (m, 1H), 7.62 (d, 2H), 7.32-7.46 (m, 3H), 7.28 (d, 1H), 7.15 (d, 1H), 4.48 (m, 1H), 3.81 (m, 2H).

Alternatively, agents of the invention may also be prepared by a reaction sequence involving a Suzuki cross-coupling reaction between an unprotected aniline boronic acid and an unprotected 4-bromo-benzoic acid, followed by sulfonamidation with appropriate sulfonyl chlorides, and coupling of an appropriate amine by means of reaction with an acid chloride intermediate, optionally followed by a deprotection step, as shown in reaction scheme 2c below:

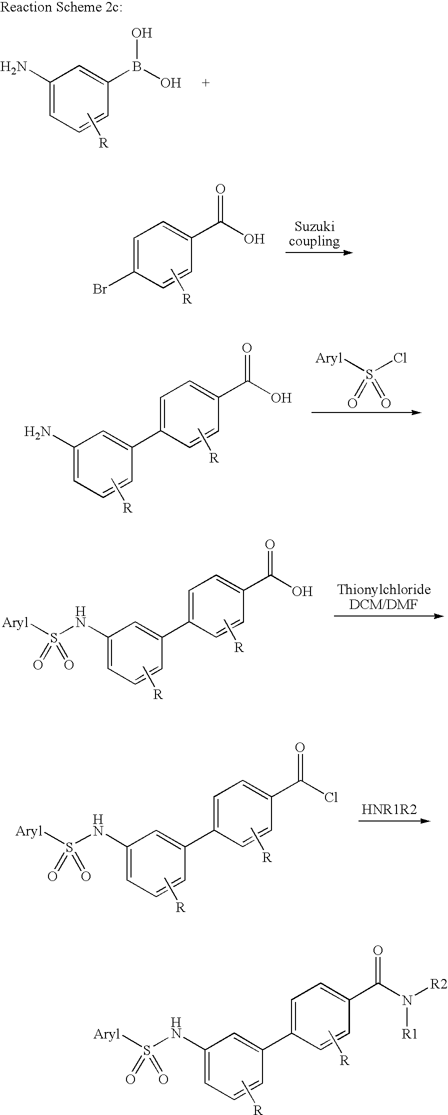

### EXAMPLE 108

### (R)-2-{[3′-(4-Chloro-2,5-dimethyl-benzenesulfonylamino)-3,5-dimethyl-biphenyl-4-carbonyl]-amino}-3-hydroxy-propionic acid methyl ester

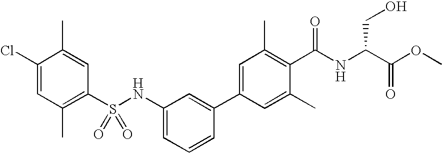

### (1) 3′-Amino-3,5-dimethyl-biphenyl-4-carboxylic acid methyl ester (27)

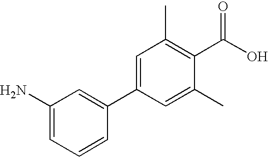

To a mixture of 4-bromo-2,6-dimethyl-benzoic acid (1.66 g, 7.23 mmol) and tetrakis-triphenylphosphinopalladium (25 mg, 0.022 mmol) in DME (200 ml) and aqueous sodium bicarbonate solution (10%, 45 ml, 50.6 mmol) is added (3-aminophenyl)-boronic acid (1.09 g, 7.95 mmol). The mixture is heated to 10° C. for 60 minutes. Upon cooling a brownish oily layer is formed which is carefully decanted. The solvents are then evaporated. Water is added and the mixture is washed with ether. The pH of the aqueous layer is adjusted to about 3 with 2N—HCl upon which a slightly sticky solid precipitates. The solid is filtered off, re-dissolved in ethyl acetate and dried over sodium sulphate. Filtration and evaporation gives the title compound 27 as a beige powder.

MS (ESI): 242 [M+H]⁺, 1H-NMR (DMSO-d6): δ (ppm) 7.27 (s, 2H), 7.1 (t, 1H), 6.84 (br s, 1H), 6.76 (d, 1H), 6.58 (m, 1H), 3.35 (br s, 2H), 2.33 (s, 3H).

### (2) 3′-(4-Chloro-2,5-dimethyl-benzenesulfonylamino)-3,5-dimethyl-biphenyl-4-carboxylic acid (28)

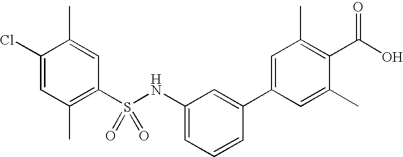

To a solution of the aniline 27 (339 mg, 1.405 mmol) in a mixture of DCM and pyridine is added 4-chloro-2,5-dimethyl-benzenesulfonyl chloride (336 mg, 1.405 mmol). The resulting mixture is stirred at room temperature for 3 hours before dilution with EtOAc (50 ml). The medium is washed three times with 2N—HCl (25 ml), water (25 ml) and brine, dried over sodium sulphate and evaporated. An orange powder of the title compound 28 is obtained.

MS (ESI): 442-444 [M−H]⁻, 1H-NMR (DMSO-d6): δ (ppm) 13.2 (br s, 1H), 10.56 (s, 1H), 7.98 (s, 1H), 7.5 (s, 1H), 7.35 (m, 2H), 7.26 (s, 1H), 7.16 (s, 2H), 7.06 (d, 1H), 2.55 (s, 3H), 2.37 (s, 3H), 2.34 (s, 6H).

### (3) (R)-2-{[3′-(4-Chloro-2,5-dimethyl-benzenesulfonylamino)-3,5-dimethyl-biphenyl-4-carbonyl]-amino}-3-hydroxy-propionic acid methyl ester

To a suspension of the acid 28 (500 mg, 1.13 mmol) in DCM (20 ml) and a catalytic amount of DMF (3 drops) is added thionylchloride (164 μl, 2.26 mmol) and the mixture is heated to reflux for about 30-60 minutes upon which all solid is dissolving. Complete formation of the acid chloride intermediate is checked by quenching an aliquot with methanol and analysing the sample as the methyl ester. The solvents are then evaporated and dried under high vacuum for about 15 minutes. The resulting foam is dissolved in THF (20 ml) and solid (R)-2-Amino-3-hydroxy-propionic acid methyl ester hydrochloride (210 mg, 1.356 mmol) is added, followed by DIEA (771 μl, 4.52 mmol). The mixture is stirred at room temperature for 16 hours. Ethyl acetate (30 ml) is then added and the mixture is washed twice with 2N—HCl, 0.5N—HCl, water, 10% sodium carbonate and brine. The organic layer is dried over sodium sulphate, filtered and evaporated to give the title compound Example 108 as a white powder. The quality of the material is usually sufficiently pure but can optionally be further purified by chromatography on silica gel (hexane/EtOAc from 10% to 80%).

MS (ESI): 543-545 [M−H]⁻, 1H-NMR (DMSO-d6): δ (ppm) 10.54 (br s, 1H), 8.61 (d, 1H), 7.96 (s, 1H), 7.49 (s, 1H), 7.29 (m, 2H), 7.23 (s, 1H), 7.11 (s, 2H), 7.05 (m, 1H), 4.93 (t, 1H), 4.53 (m, 1H), 3.74 (m, 2H), 3.68 (s, 3H), 2.53 (s, 3H), 2.35 (s, 3H), 2.3 (s, 6H).

The following ester derivatives are prepared according to the procedure described in step 3 of Example 108 using the intermediate acid 28 and the appropriate amino acid esters:

### EXAMPLE 109

### (S)-2-{[3′-(4-Chloro-2,5-dimethyl-benzenesulfonylamino)-3,5-dimethyl-biphenyl-4-carbonyl]-amino}-3-hydroxy-propionic acid ethyl ester

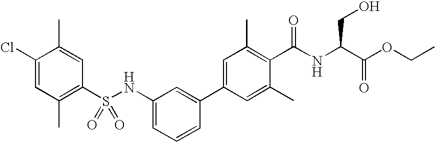

MS (ESI): 557-559 [M−H]⁻, 1H-NMR (DMSO-d6): δ (ppm) 10.55 (br s, 1H), 8.6 (d, 1H), 7.96 (s, 1H), 7.5 (s, 1H), 7.29 (m, 2H), 7.23 (s, 1H), 7.11 (s, 2H), 7.05 (m, 1H), 4.91 (t, 1H), 4.52 (m, 1H), 4.13 (m, 2H), 3.73 (m, 2H), 2.53 (s, 3H), 2.35 (s, 3H), 2.3 (s, 6H), 1.22 (t, 3H).

### EXAMPLE 110

### (S)-2-{[3′-(4-Chloro-2,5-dimethyl-benzenesulfonylamino)-3,5-dimethyl-biphenyl-4-carbonyl]-amino}-propionic acid methyl ester

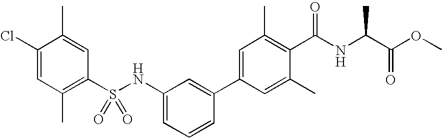

MS (ESI): 527-529 [M−H]⁻, 1H-NMR (DMSO-d6): δ (ppm) 10.51 (br s, 1H), 8.77 (d, 1H), 7.96 (s, 1H), 7.49 (s, 1H), 7.29 (m, 2H), 7.23 (s, 1H), 7.11 (s, 2H), 7.05 (m, 1H), 4.47 (m, 1H), 3.67 (s, 3H), 2.53 (s, 3H), 2.35 (s, 3H), 2.29 (s, 6H), 1.35 (d, 3H).

### EXAMPLE 111

### (S)-2-{[3′-(4-Chloro-2,5-dimethyl-benzenesulfonylamino)-3,5-dimethyl-biphenyl-4-carbonyl]-amino}-propionic acid ethyl ester

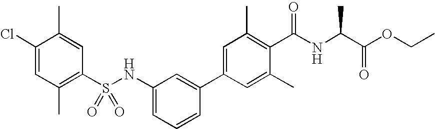

MS (ESI): 543-545 [M+H]⁺, 1H-NMR (DMSO-d6): δ (ppm) 10.56 (br s, 1H), 8.77 (d, 1H), 7.97 (s, 1H), 7.51 (s, 1H), 7.3 (m, 2H), 7.24 (s, 1H), 7.12 (s, 2H), 7.06 (m, 1H), 4.46 (m, 1H), 4.13 (m, 2H), 2.54 (s, 3H), 2.37 (s, 3H), 2.31 (s, 6H), 1.36 (d, 3H), 1.23 (t, 3H).

### EXAMPLE 112

### (S)-2-{[3′-(4-Chloro-2,5-dimethyl-benzenesulfonylamino)-3,5-dimethyl-biphenyl-4-carbonyl]-amino}-3-hydroxy-propionic acid methyl ester

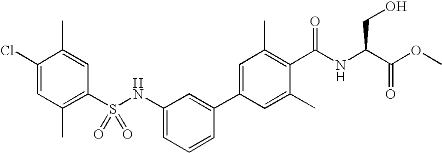

MS (ESI): 543-545 [M−H]⁻, 1H-NMR (DMSO-d6): δ (ppm) 10.54 (br s, 1H), 8.61 (d,1H), 7.96 (s, 1H), 7.49 (s, 1H), 7.29 (m, 2H), 7.23 (s, 1H), 7.11 (s, 2H), 7.05 (m, 1H), 4.93 (t, 1H), 4.53 (m, 1H), 3.74 (m, 2H), 3.68 (s, 3H), 2.53 (s, 3H), 2.35 (s, 3H), 2.3 (s, 6H).

### EXAMPLE 113

### (R)-2-{[3′-(4-Chloro-2,5-dimethyl-benzenesulfonylamino)-3,5-dimethyl-biphenyl-4-carbonyl]-amino}-propionic acid methyl ester

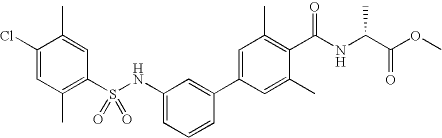

MS (ESI): 527-529 [M−H]⁻, 1H-NMR (DMSO-d6): δ (ppm) 10.51 (br s, 1H), 8.77 (d, 1H), 7.96 (s, 1H), 7.49 (s, 1H), 7.29 (m, 2H), 7.23 (s, 1H), 7.11 (s, 2H), 7.05 (m, 1H), 4.47 (m, 1H), 3.97 (s, 3H), 2.53 (s, 3H), 2.35 (s, 3H), 2.29 (s, 6H), 1.35 (d, 3H).

### EXAMPLE 114

### (S)-2-{[3′-(4-Chloro-2,5-dimethyl-benzenesulfonylamino)-3,5-dimethyl-biphenyl-4-carbonyl]-amino}-butyric acid tert-butyl ester

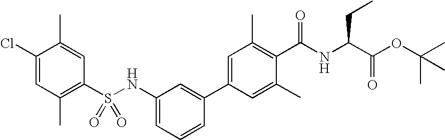

MS (ESI): 583-585 [M−H]⁻

### EXAMPLE 115

### (S)-2-{[3′-(4-Chloro-2,5-dimethyl-benzenesulfonylamino)-3,5-dimethyl-biphenyl-4-carbonyl]-amino}-3-methoxy-propionic acid methyl ester

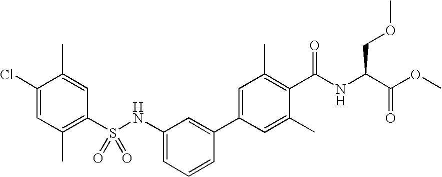

MS (ESI): 559-561 [M+H]⁺, 1H-NMR (CDCl₃): δ (ppm) 7.88 (s, 1H), 7.38 (m, 3H), 7.14 (s, 1H), 7.09 (s, 2H), 7.01 (m, 1H), 6.89 (s, 1H), 6.55 (d, 1H), 5.01 (m, 1H), 3.95 (dd, 1H), 3.83 (s, 3H), 3.73 (dd, 1H), 3.35 (s, 3H), 2.58 (s, 3H), 2.41 (s, 6H), 2.35 (s, 3H).

### EXAMPLE 116

### {[3′-(4-Chloro-2,5-dimethyl-benzenesulfonylamino)-3,5-dimethyl-biphenyl-4-carbonyl]-amino}-acetic acid ethyl ester

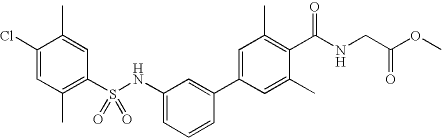

MS (ESI): 527-529 [M−H]⁻

### EXAMPLE 117

### (S)-2-{[3′-(4-Chloro-2,5-dimethyl-benzenesulfonylamino)-3,5-dimethyl-biphenyl-4-carbonyl]-methyl-amino}-3-hydroxy-propionic acid methyl ester

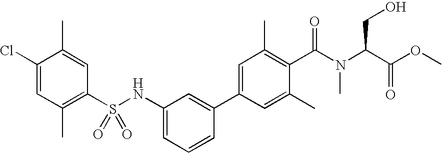

The title compound is a mixture of rotamers.

MS (ESI): 557-559 [M−H]⁻.

### EXAMPLE 118

### (S)-2-{[3′-(4-Chloro-2,5-dimethyl-benzenesulfonylamino)-3,5-dimethyl-biphenyl-4-carbonyl]-methyl-amino}-propionic acid methyl ester

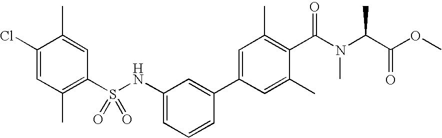

The title compound is a mixture of rotamers.

MS (ESI): 541-543 [M−H]⁻.

### EXAMPLE 119

### 2-{[3′-(4-Chloro-2,5-dimethyl-benzenesulfonylamino)-3,5-dimethyl-biphenyl-4-carbonyl]-amino}-2-methyl-propionic acid methyl ester

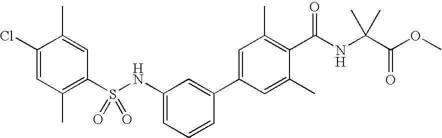

MS (ESI): 541-543 [M−H]⁻, 1H-NMR (DMSO-d₆): δ (ppm) 10.54 (br s, 1H), 8.76 (s, 1H), 7.96 (s, 1H), 7.49 (s, 1H), 7.29 (m, 2H), 7.22 (s, 1H), 7.1 (s, 2H), 7.04 (m, 1H), 3.64 (s, 3H), 2.53 (s, 3H), 2.35 (s, 3H), 2.29 (s, 6H), 1.43 (s, 6H).

### EXAMPLE 120

### (S)-3-tert-Butoxycarbonylamino-2-{[3′-(4-chloro-2,5-dimethyl-benzenesulfonylamino)-3,5-dimethyl-biphenyl-4-carbonyl]-amino}-propionic acid methyl ester

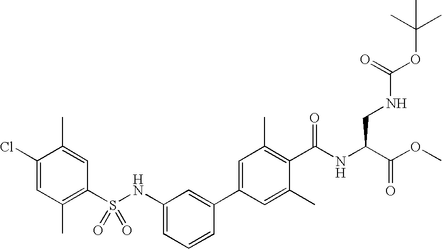

MS (ESI): 642-644 [M−H]⁻, 1H-NMR (DMSO-d₆): δ (ppm) 10.55 (br s,1H), 8.59 (d, 1H), 7.97 (s, 1H), 7.49 (s, 1H), 7.29 (m, 2H), 7.22 (s, 1H), 7.12 (s, 2H), 7.05 (m, 1H), 6.82 (t, 1H), 4.56 (m, 1H), 3.66 (s, 3H), 3.35 (m, 2H), 2.53 (s, 3H), 2.35 (s, 3H), 2.29 (s, 6H), 1.37 (s, 9H).

### EXAMPLE 121

### (R)-3-tert-Butoxycarbonylamino-2-{[3′-(4-chloro-2,5-dimethyl-benzenesulfonylamino)-3,5-dimethyl-biphenyl-4-carbonyl]-amino}-propionic acid methyl ester

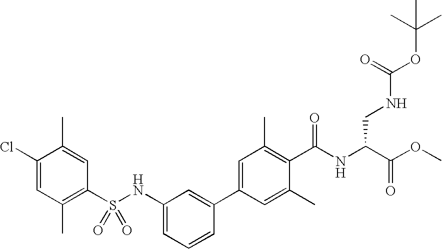

MS (ESI): 642-644 [M−H]⁻, 1H-NMR (DMSO-d₆): δ (ppm) 10.55 (br s,1H), 8.59 (d, 1H), 7.97 (s, 1H), 7.49 (s, 1H), 7.29 (m, 2H), 7.22 (s, 1H), 7.12 (s, 2H), 7.05 (m, 1H), 6.82 (t, 1H), 4.56 (m, 1H), 3.66 (s, 3H), 3.35 (m, 2H), 2.53 (s, 3H), 2.35 (s, 3H), 2.29 (s, 6H), 1.37 (s, 9H).

### EXAMPLE 122

### (S)-3-Amino-2-{[3′-(4-chloro-2,5-dimethyl-benzenesulfonylamino)-3,5-dimethyl-biphenyl-4-carbonyl]-amino}-propionic acid methyl ester hydrochloride

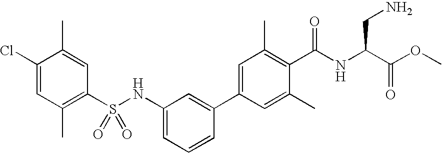

The title compound is obtained as the hydrochloride salt by standard Boc-cleavage of Example 120 with excess of HCl in dioxane at room temperature followed by evaporation.

MS (ESI): 542-544 [M−H]⁻, 1H-NMR (DMSO-d₆): δ (ppm) 10.5 (brs, 1H), 8.91 (d, 1H), 8.15 (brs, 2H), 7.97 (s, 1H), 7.5 (s, 1H), 7.3 (m, 2H), 7.25 (s, 1H), 7.14 (s, 2H), 7.06 (m, 1H), 4.77 (m, 1H), 3.74 (s, 3H), 3.3 (dd,1H, overlapping with water signal), 3.13 (dd,1H), 2.54 (s, 3H), 2.36 (s, 3H), 2.32 (s, 6H).

### EXAMPLE 123

### (R)-3-Amino-2-{[3′-(4-chloro-2,5-dimethyl-benzenesulfonylamino)-3,5-dimethyl-biphenyl-4-carbonyl]-amino}-propionic acid methyl ester hydrochloride

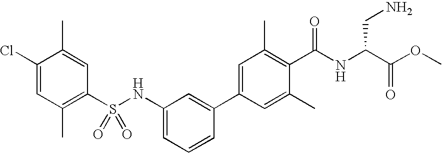

The title compound is obtained as the hydrochloride salt by standard Boc-cleavage of Example 121 with excess of HCl in dioxane at room temperature followed by evaporation.

MS (ESI): 542-544 [M−H]⁻, 1H-NMR (DMSO-d₆): δ (ppm) 10.5 (brs, 1H), 8.91 (d, 1H), 8.15 (brs, 2H), 7.97 (s, 1H), 7.5 (s, 1H), 7.3 (m, 2H), 7.25 (s, 1H), 7.14 (s, 2H), 7.06 (m, 1H), 4.77 (m, 1H), 3.74 (s, 3H), 3.3 (dd, 1H, overlapping with water signal), 3.13 (dd, 1H), 2.54 (s, 3H), 2.36 (s, 3H), 2.32 (s, 6H).

### EXAMPLE 124

### 3-{[3′-(4-Chloro-2,5-dimethyl-benzenesulfonylamino)-3,5-dimethyl-biphenyl-4-carbonyl]-amino}-azetidine-1,3-dicarboxylic acid 1-tert-butyl ester 3-ethyl ester

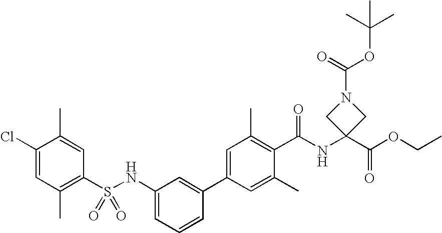

The synthesis of this compound is accomplished analogously to the synthesis of Example 108, using 3-Amino-azetidine-1,3-dicarboxylic acid 1-tert-butyl ester 3-ethyl ester (29) (preparation see below) in step 3.

MS (ESI): 670-672 [M+H]⁺, 1H-NMR (DMSO-d6): δ (ppm) 10.59 (br s, 1H), 9.60 (s, 1H), 7.99 (s, 1H), 7.50 (s, 1H), 7.33 (t, 1H), 7.28 (d, 1H), 7.23 (s, 1H), 7.16 (s, 2H), 7.06 (d, 1H), 4.31 (br d, 2H), 4.21 (q, 2H), 3.97 (d, 2H), 2.52 (s, 3H), 2.38 (s, 3H), 2.33 (s, 6H), 1.41 (s, 9H), 1.25 (t, 3H).

### Synthesis of 3-Amino-azetidine-1,3-dicarboxylic acid 1-tert-butyl ester 3-ethyl ester (29)

### (1) 3-Benzyloxycarbonylamino-azetidine-1,3-dicarboxylic acid 1-tert-butyl ester 3-ethyl ester (30)

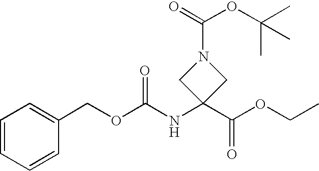

1-Benzyl-azetidine-3,3-dicarboxylic acid diethyl ester (Lit.: Synth. Commun. 2003, 33, 3347-3353) (2.00 g, 6.86 mmol) is dissolved in EtOH (23 ml) and 4M-HCl in dioxane (1.72 ml) is added followed by palladium hydroxide on charcoal (0.36 g, 3.43 mmol). The reaction mixture is hydrogenated for 15 hours. The mixture is filtrated over hyflo and the filtrate is concentrated.

To the crude Azetidine-3,3-dicarboxylic acid diethyl ester (1.38 g, 6.86 mmol) dissolved in THF (23 ml) is added BOC₂O (1.65 g, 7.54 mmol), DIPEA (3 ml, 21 mmol) and a catalytic amount of DMAP (82.8 mg, 0.68 mmol). The mixture is stirred for 15 hours at room temperature. Water (100 ml) is added and the organic phase is separated. The aqueous layer is extracted with EtOAc (3×). The combined organic layers are dried over sodium sulfate, filtered and evaporated.

Crude Azetidine-1,3,3-tricarboxylic acid 1-tert-butyl ester 3,3-diethyl ester (1.57 g, 3.12 mmol) is dissolved in EtOH (21 ml) and treated with 1N—NaOH solution. After stirring for 39 hours the mixture is diluted with water (20 ml) and the pH is adjusted to 1 by adding 0.5 N—HCl solution. After extraction with EtOAc (3×100 ml), the organic layer is dried over sodium sulfate and concentrated.

Crude Azetidine-1,3,3-tricarboxylic acid 1-tert-butyl ester 3-ethyl ester (990 mg, 3.63 mmol) is dissolved in toluene (36 ml). Diphenylphosphoryl azide (0.93 ml, 4.31 mmol) and triethylamine (0.60 ml, 4.31 mmol) is added and the mixture is heated at 115° C. for 2 hours. The mixture is cooled to RT and benzyl alcohol (0.78 ml, 7.25 mmol) is added. The mixture is heated at 115° C. for 2.5 h. The cooled mixture is diluted with EtOAc, washed with sodium bicarbonate and brine, dried and evaporated. 3-Benzyloxycarbonylamino-azetidine-1,3-dicarboxylic acid 1-tert-butyl ester 3-ethyl ester 30 is obtained after silica gel chromatography using cyclohexane/EtOAc.

MS (ESI): 515-517 [M−H]⁻, 1H-NMR (DMSO-d6): δ (ppm) 8.50 (s, 1H), 7.41-7.28 (m, 5H), 5.06 (s, 2H), 4.15 (q, 2H), 4.18-4.10 (m, 2H), 3.92-3.80 (m, 2H), 1.47 (s, 9H), 1.16 (t, 3H).

### (2) 3-Amino-azetidine-1,3-dicarboxylic acid 1-tert-butyl ester 3-ethyl ester (29)

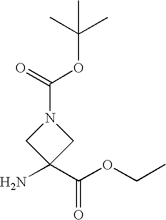

The ester 30 (5.22 g, 13.8 mmol) is dissolved in EtOH(50 ml) and cyclohexene (84 ml, 828 mmol). Palladium on charcoal (0.73 g) is added and the mixture is refluxed for 2.5 hours, cooled and filtrated over hyflo and evaporated to yield the title compound.

MS (ESI): 489 [2M+H]⁺, 1H-NMR (DMSO-d6): δ (ppm) 4.15 (q, 2H), 4.10-3.98 (m, 2H), 3.70-3.56 (m, 2H), 2.46 (br s, 2H), 1.40 (s, 9H), 1.23 (t, 3H).

### EXAMPLE 125

### 4-{[3′-(4-Chloro-2,5-dimethyl-benzenesulfonylamino)-3,5-dimethyl-biphenyl-4-carbonyl]-amino}-1-methyl-piperidine-4-carboxylic acid methyl ester

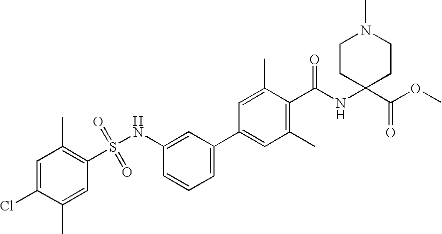

The synthesis of this compound is accomplished analogously to the synthesis of Example 108, using 4-Amino-1-methyl-piperidine-4-carboxylic acid methyl ester (J. Med. Chem. 2007, 50, 2341-2351) in step 3.

MS (ESI): 598-600 [M+H]⁺.

### EXAMPLE 126

### 4-{[3′-(4-Chloro-2,5-dimethyl-benzenesulfonylamino)-3,5-dimethyl-biphenyl-4-carbonyl]-amino}-tetrahydro-pyran-4-carboxylic acid ethyl ester

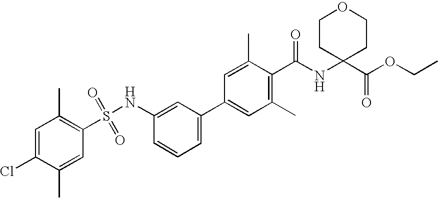

The synthesis of this compound is accomplished analogously to the synthesis of Example 108, using 4-Amino-tetrahydro-pyran-4-carboxylic acid ethyl ester in step 3.

MS (ESI): 599-601 [M+H]⁺, 1H-NMR (DMSO-d6): δ (ppm) 10.54 (brs, 1H), 8.85 (brs, 1H), 7.96 (s, 1H), 7.50 (s, 1H), 7.31 (t, 1H), 7.28 (d, 1H), 7.23 (s, 1H), 7.12 (s, 2H), 7.06 (d, 1H), 4.13 (q, 2H), 3.73 (td, 2H), 3.65 (dt, 2H), 2.53 (s, 3H), 2.36 (s, 3H), 2.35 (m, 4H), 1.22 (t, 3H).

### EXAMPLE 127

### 1-{[3′-(4-Chloro-2,5-dimethyl-benzenesulfonylamino)-3,5-dimethyl-biphenyl-4-carbonyl]-amino}-cyclobutanecarboxylic acid ethyl ester

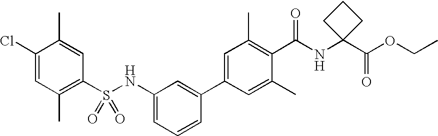

The synthesis of this compound is accomplished analogously to the synthesis of Example 108, using 1-Amino-cyclobutanecarboxylic acid ethyl ester in step 3.

MS (ESI): 569-571 [M+H]⁺, 1H-NMR (DMSO-d6): δ (ppm) 10.54 (br s, 1H), 9.13 (s, 1H), 7.96 (s, 1H), 7.50 (s, 1H), 7.31 (t, 1H), 7.26 (d, 1H), 7.23 (s, 1H), 7.12 (s, 2H), 7.06 (d, 1H), 4.13 (d, 2H), 2.63-2.49 (m, 2H), 2.54 (s, 3H), 2.36 (s, 3H), 2.32 (s, 6H), 2.29-2.19 (m, 2H), 2.02-1.85 (dd, 2H), 122 (t, 3H).

### EXAMPLE 128

### 1-{[3′-(4-Chloro-2,5-dimethyl-benzenesulfonylamino)-3,5-dimethyl-biphenyl-4-carbonyl]-amino}-cyclopropanecarboxylic acid ethyl ester

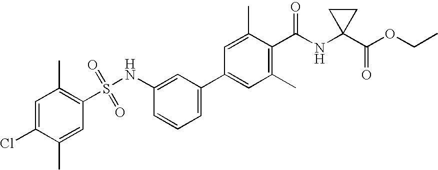

The synthesis of this compound is accomplished analogously to the synthesis of Example 108, using 1-Amino-cyclopropanecarboxylic acid ethyl ester in step 3.

MS (ESI): 555-557 [M+H]⁺, 1H-NMR (DMSO-d6): δ (ppm) 10.55 (br s, 1H), 8.94 (s, 1H), 7.97 (s, 1H), 7.51 (s, 1H), 7.32 (t, 1H), 7.27 (d, 1H), 7.24 (s, 1H), 7.11 (s, 2H), 7.05 (d, 1H), 4.12 (q, 2H), 2.54 (s, 3H), 2.37 (s, 3H), 2.31 (s, 6H), 1.47 (dd, 2H), 1.22 (t, 3H), 1.14 (dd, 2H).

### EXAMPLE 129

### 3-{[3′-(4-Chloro-2,5-dimethyl-benzenesulfonylamino)-3,5-dimethyl-biphenyl-4-carbonyl]-amino}-azetidine-3-carboxylic acid methyl ester

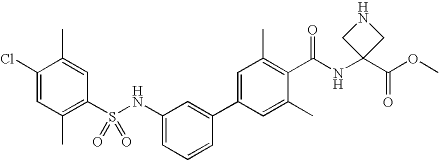

A solution of Example 145 (1.02 g, 1.50 mmol) and thionyl chloride (0.218 ml, 3.0 mmol) in MeOH (15 ml) is heated at 60° C. for 6.5 hours. The solvent is evaporated to give the title compound as a sufficiently pure white powder.

MS (ESI): 556-558 [M+H]⁺, 1H-NMR (DMSO-d6): δ (ppm) 10.61 (br s, 1H), 9.84 (s, 1H), 9.78 (br s, 1H), 9.45 (br s, 1H), 8.00 (s, 1H), 7.50 (s, 1H), 7.32 (t, 1H), 7.28 (d, 1H), 7.25 (s, 1H), 7.15 (s, 2H), 7.06 (d, 1H), 4.51 (d, 2H), 4.10 (d, 2H), 3.78 (s, 3H), 2.56 (s, 3H), 2.37 (s, 9H).

### EXAMPLE 130

### 3-{[3′-(4-Chloro-2,5-dimethyl-benzenesulfonylamino)-3,5-dimethyl-biphenyl-4-carbonyl]-amino}-1-methyl-azetidine-3-carboxylic acid methyl ester

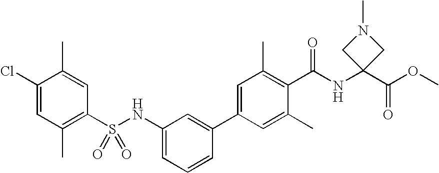

The synthesis of this compound is accomplished by reductive amination of Example 129 and aqueous formaldehyde according to the procedure described in step 3 of Example 162.

MS (ESI): 570-572 [M+H]⁺, 1H-NMR (DMSO-d6): δ (ppm) 10.58 (s, 1H), 9.43 (s, 1H), 7.99 (s, 1H), 7.50 (s, 1H), 7.31 (t, 1H), 7.25 (d, 1H), 7.22 (s, 1H), 7.10 (s, 2H), 7.03 (d, 1H), 3.70 (s, 3H), 3.61 (d, 2H), 3.32 (d, 2H), 2.52 (s, 3H), 2.33 (s, 3H), 2.30 (s, 6H), 2.25 (s, 3H).

The free carboxylic acid derivatives of the above esters are obtained by LiOH-hydrolysis in THF as described in step 2 of Example 48

### EXAMPLE 131

### (S)-2-{[3′-(4-Chloro-2,5-dimethyl-benzenesulfonylamino)-3,5-dimethyl-biphenyl-4-carbonyl]-amino}-3-hydroxy-propionic acid

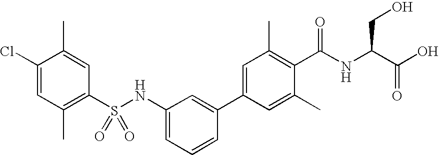

The title compound is obtained by hydrolysis of Example 112.

MS (ESI): 529-531 [M−H]⁻, 1H-NMR (DMSO-d6): δ (ppm) 12.62 (br s, 1H), 10.55 (br s, 1H), 8.46 (d, 1H), 7.98 (s, 1H), 7.51 (s, 1H), 7.31 (m, 2H), 7.24 (s, 1H), 7.12 (s, 2H), 7.06 (m, 1H), 4.8 (br s, 1H), 4.49 (m, 1H), 3.75 (m, 2H), 2.55 (s, 3H), 2.37 (s, 3H), 2.32 (s, 6H).

### EXAMPLE 132

### (S)-2-{[3′-(4-Chloro-2,5-dimethyl-benzenesulfonylamino)-3,5-dimethyl-biphenyl-4-carbonyl]-amino}-propionic acid

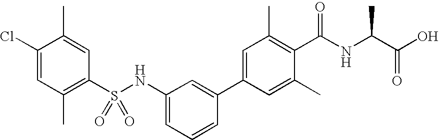

The title compound is obtained by hydrolysis of Example 110.

MS (ESI): 513-515 [M−H]⁻, 1H-NMR (DMSO-d6): δ (ppm) 12.54 (br s, 1H), 10.56 (br s, 1H), 8.66 (d, 1H), 7.96 (s, 1H), 7.46 (s, 1H), 7.31 (m, 2H), 7.24 (s, 1H), 7.11 (s, 2H), 7.02 (m, 1H), 4.41 (m, 1H), 2.55 (s, 3H), 2.37 (s, 3H), 2.31 (s, 6H), 1.34 (d, 3H).

### EXAMPLE 133

### (R)-2-{[3′-(4-Chloro-2,5-dimethyl-benzenesulfonylamino)-3,5-dimethyl-biphenyl-4-carbonyl]-amino}-3-hydroxy-propionic acid

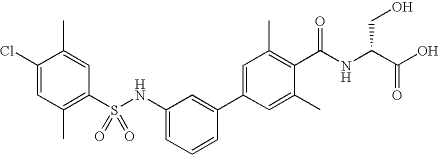

The title compound is obtained by hydrolysis of Example 108.

MS (ESI): 529-531 [M−H]⁻, 1H-NMR (DMSO-d6): δ (ppm) 12.62 (br s, 1H), 10.55 (br s, 1H), 8.46 (d, 1H), 7.98 (s, 1H), 7.51 (s, 1H), 7.31 (m, 2H), 7.24 (s, 1H), 7.12 (s, 2H), 7.06 (m, 1H), 4.8 (br s, 1H), 4.49 (m, 1H), 3.75 (m, 2H), 2.55 (s, 3H), 2.37 (s, 3H), 2.32 (s, 6H).

### EXAMPLE 134

### (R)-2-{[3′-(4-Chloro-2,5-dimethyl-benzenesulfonylamino)-3,5-dimethyl-biphenyl-4-carbonyl]-amino}-propionic acid

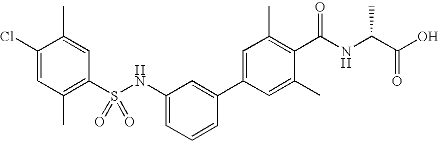

The title compound is obtained by hydrolysis of Example 113.

MS (ESI): 513-515 [M−H]⁻, 1H-NMR (DMSO-d6): δ (ppm) 12.54 (br s, 1H), 10.56 (br s, 1H), 8.66 (d, 1H), 7.96 (s, 1H), 7.46 (s, 1H), 7.31 (m, 2H), 7.24 (s, 1H), 7.11 (s, 2H), 7.02 (m, 1H), 4.41 (m, 1H), 2.55 (s, 3H), 2.37 (s, 3H), 2.31 (s, 6H), 1.34 (d, 3H).

### EXAMPLE 135

### (S)-2-{[3′-(4-Chloro-2,5-dimethyl-benzenesulfonylamino)-3,5-dimethyl-biphenyl-4-carbonyl]-amino}-butyric acid

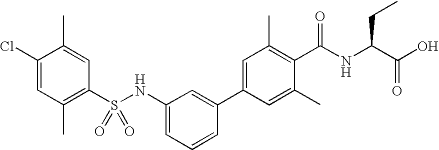

The title compound is obtained by TFA cleavage of Example 114.

MS (ESI): 527-529 [M−H]⁻, 1H-NMR (DMSO-d6): δ (ppm) 10.8 (v br s, 1H), 8.07 (br s, 1H), 7.95 (s, 1H), 7.45 (s, 1H), 7.26 (m, 1H), 7.19 (m, 2H), 7.09 (s, 2H), 7.0 (m, 1H), 4.21 (m, 1H), 2.53 (s, 3H), 2.34 (s, 3H), 2.29 (s, 6H), 1.86 (m, 1H), 1.67 (m, 1H), 0.93 (t, 3H).

### EXAMPLE 136

### (S)-2-{[3′-(4-Chloro-2,5-dimethyl-benzenesulfonylamino)-3,5-dimethyl-biphenyl-4-carbonyl]-amino}-3-methoxy-propionic acid

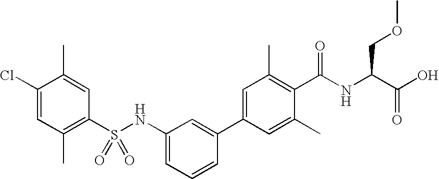

The title compound is obtained by hydrolysis of Example 115.

MS (ESI): 545-547 [M−H]⁻, 1H-NMR (DMSO-d6): δ (ppm) 12. 76 (v br s, 1H), 10.54 (br s, 1H), 8.66 (d, 1H), 7.96 (s, 1H), 7.5 (s, 1H), 7.29 (m, 2H), 7.23 (s, 1H), 7.1 (s, 2H), 7.05 (m, 1H), 4.63 (m, 1H), 3.66 (m, 2H), 3.28 (s, 3H), 2.53 (s, 3H), 2.35 (s, 3H), 2.29 (s, 6H).

### EXAMPLE 137

### {[3′-(4-Chloro-2,5-dimethyl-benzenesulfonylamino)-3,5-dimethyl-biphenyl-4-carbonyl]-amino}-acetic acid

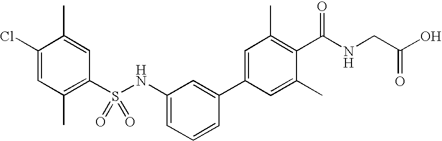

The title compound is obtained by hydrolysis of Example 116.

MS (ESI): 499-501 [M−H]⁻, 1H-NMR (DMSO-d6): δ (ppm) 12.4 (v br s, 1H), 10.55 (v br s, 1H), 8.63 (t, 1H), 7.96 (s, 1H), 7.49 (s, 1H), 7.3 (m, 2H), 7.23 (s, 1H), 7.11 (s, 2H), 7.05 (m, 1H), 3.9 (d, 2H), 2.53 (s, 3H), 2.35 (s, 3H), 2.3 (s, 6H).

### EXAMPLE 138

### (S)-2-{[3′-(4-Chloro-2,5-dimethyl-benzenesulfonylamino)-3,5-dimethyl-biphenyl-4-carbonyl]-methyl-amino}-3-hydroxy-propionic acid

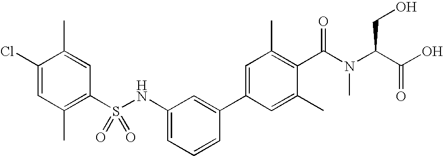

The title compound is obtained as a mixture of rotamers (LC-MS spectrum) by hydrolysis of Example 117.

MS (ESI): 545-547 [M+H]⁺, 1H-NMR (DMSO-d6): δ (ppm) 12.8 (v brs, 1H), 10.56 (brs, 1H), 7.99 (s, 1H), 7.51 (s, 1H), 7.3 (m, 2H), 7.26 (s, 1H), 7.16 (s, 2H), 7.05 (m, 1H), 5.13 (m, 1H), 3.94 (m, 2H), 2.76 (s, 3H), 2.55 (s, 3H), 2.37 (s, 3H), 2.27 (s, 3H), 2.25 (s, 3H).

### EXAMPLE 139

### (S)-2-{[3′-(4-Chloro-2,5-dimethyl-benzenesulfonylamino)-3,5-dimethyl-biphenyl-4-carbonyl]-methyl-amino}-propionic acid

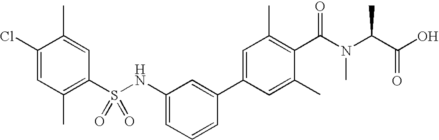

The title compound is obtained as a mixture of rotamers (LC-MS spectrum) by hydrolysis of Example 118.

MS (ESI): 529-531 [M+H]⁺, 1H-NMR (DMSO-d6): δ (ppm) 12.7 (v brs, 1H), 10.56 (br s, 1H), 7.99 (s, 1H), 7.51 (s, 1H), 7.32 (m, 2H), 7.27 (s, 1H), 7.16 (s, 2H), 7.06 (m, 1H), 5.05 (m, 1H), 2.7 (s, 3H), 2.55 (s, 3H), 2.37 (s, 3H), 2.25 (s, 3H), 2.22 (s, 3H), 1.42 (d, 3H).

### EXAMPLE 140

### (S)-3-tert-Butoxycarbonylamino-2-{[3′-(4-chloro-2,5-dimethyl-benzenesulfonylamino)-3,5-dimethyl-biphenyl-4-carbonyl]-amino}-propionic acid

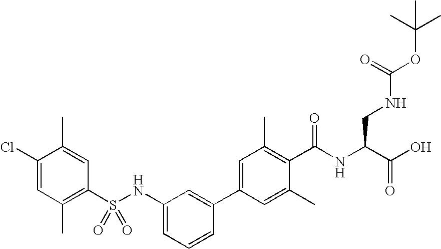

The title compound is obtained by hydrolysis of Example 120.

MS (ESI): 628-630 [M−H]⁻, 1H-NMR (DMSO-d6): δ (ppm) 12.7 (v br s, 1H), 10.56 (br s,1H), 8.42 (brd, 1H), 7.98 (s, 1H), 7.51 (s, 1H), 7.31 (m, 2H), 7.23 (s, 1H), 7.12 (s, 2H), 7.06 (m, 1H), 6.75 (m, 1H), 4.51 (m, 1H), 3.35 (m, 2H, overlapping with water signal), 2.55 (s, 3H), 2.37 (s, 3H), 2.31 (s, 6H), 1.39 (s, 9H).

### EXAMPLE 141

### (R)-3-tert-Butoxycarbonylamino-2-{[3′-(4-chloro-2,5-dimethyl-benzenesulfonylamino)-3,5-dimethyl-biphenyl-4-carbonyl]-amino}-propionic acid

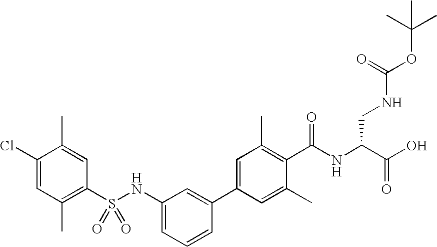

The title compound is obtained by hydrolysis of Example 121.

MS (ESI): 628-630 [M−H]⁻, 1H-NMR (DMSO-d6): δ (ppm) 12.7 (v br s, 1H), 10.56 (br s,1H), 8.42 (brd, 1H), 7.98 (s, 1H), 7.51 (s, 1H), 7.31 (m, 2H), 7.23 (s, 1H), 7.12 (s, 2H), 7.06 (m, 1H), 6.75 (m, 1H), 4.51 (m, 1H), 3.35 (m, 2H, overlapping with water signal), 2.55 (s, 3H), 2.37 (s, 3H), 2.31 (s, 6H), 1.39 (s, 9H).

### EXAMPLE 142

### (S)-3-Amino-2-{[3′-(4-chloro-2,5-dimethyl-benzenesulfonylamino)-3,5-dimethyl-biphenyl-4-carbonyl]-amino}-propionic acid

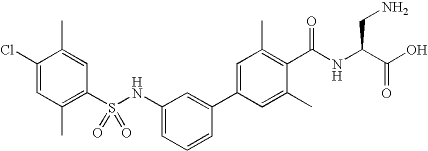

The title compound is obtained by hydrolysis of Example 122.

MS (ESI): 528-530 [M−H]⁻, 1H-NMR (DMSO-d6): δ (ppm) 10.57 (br s, 1H), 8.77 (d, 1H), 8.1 (v brs, 2H), 7.97 (s, 1H), 7.5 (s, 1H), 7.31 (m, 2H), 7.25 (s, 1H), 7.13 (s, 2H), 7.06 (m, 1H), 4.68 (m, 1H), 3.3 (m, 1H, overlapping with water signal), 3.09 (m, 1H), 2.53 (s, 3H), 2.35 (s, 3H), 2.33 (s, 6H).

### EXAMPLE 143

### (R)-3-Amino-2-{[3′-(4-chloro-2,5-dimethyl-benzenesulfonylamino)-3,5-dimethyl-biphenyl-4-carbonyl]-amino}-propionic acid

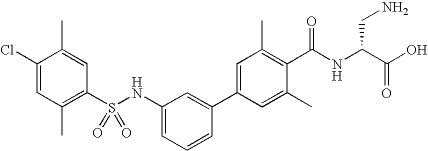

The title compound is obtained by hydrolysis of Example 123.

MS (ESI): 528-530 [M−H]⁻, 1H-NMR (DMSO-d6): δ (ppm) 10.57 (br s, 1H), 8.77 (d, 1H), 8.1 (v brs, 2H), 7.97 (s, 1H), 7.5 (s, 1H), 7.31 (m, 2H), 7.25 (s, 1H), 7.13 (s, 2H), 7.06 (m, 1H), 4.68 (m, 1H), 3.3 (m, 1H, overlapping with water signal), 3.09 (m, 1H), 2.53 (s, 3H), 2.35 (s, 3H), 2.33 (s, 6H).

### EXAMPLE 144

### 3-{[3′-(4-Chloro-2,5-dimethyl-benzenesulfonylamino)-3,5-dimethyl-biphenyl-4-carbonyl]-amino}-azetidine-1,3-dicarboxylic acid mono-tert-butyl ester

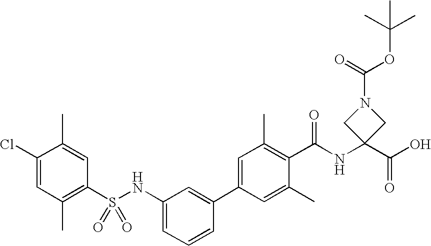

The title compound is obtained by hydrolysis of Example 124.

MS (ESI): 642-644 [M+H]⁺, 1H-NMR (DMSO-d6): δ (ppm) 13.18 (br s,1H), 10.58 (br s,1H), 9.46 (s, 1H), 7.99 (s, 1H), 7.50 (s, 1H), 7.33 (t, 1H), 7.28 (d, 1H), 7.24 (s, 1H), 7.13 (s, 2H), 7.05 (d, 1H), 4.30 (br d, 2H), 3.96 (d, 2H), 2.54 (s, 3H), 2.38 (s, 3H), 2.32 (s, 6H), 1.40 (s, 9H).

### EXAMPLE 145

### 3-{[3′-(4-Chloro-2,5-dimethyl-benzenesulfonylamino)-3,5-dimethyl-biphenyl-4-carbonyl]-amino}-azetidine-3-carboxylic acid

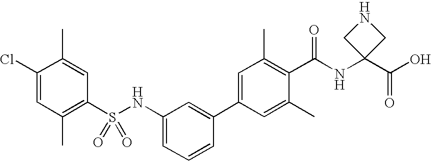

The title compound is obtained as the hydrochloride salt by standard Boc-cleavage of Example 144 with excess of 4M HCl in dioxane at room temperature followed by evaporation.

MS (ESI): 542-544 [M+H]⁺, 1H-NMR (DMSO-d6): δ (ppm) 13.68 (br s,1H), 10.60 (br s,1H), 9.44 (brs, 3H), 8.00 (s, 1H), 7.51 (s, 1H), 7.32 (t, 1H), 7.28 (d, 1H), 7.25 (s, 1H), 7.14 (s, 2H), 7.07 (d, 1H), 4.42 (d, 2H), 4.12 (d, 2H), 2.57 (s, 3H), 2.37 (s, 9H).

### EXAMPLE 146

### 4-{[3′-(4-Chloro-2,5-dimethyl-benzenesulfonylamino)-3,5-dimethyl-biphenyl-4-carbonyl]-amino}-tetrahydro-pyran-4-carboxylic acid

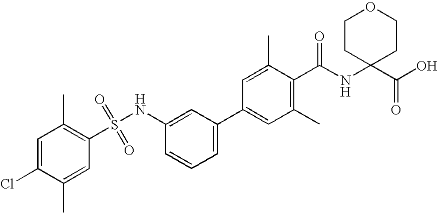

The title compound is obtained by hydrolysis of Example 126.

MS (ESI): 571-573 [M+H]⁺, 1H-NMR (DMSO-d6): δ (ppm) 11.04 (br s,1H), 8.46 (br s, 1H), 7.94 (s, 1H), 7.43 (s, 1H), 7.23 (t, 1H), 7.17 (s, 1H), 7.14 (d, 1H), 7.08 (s, 2H), 6.97 (d, 1H), 3.73 (dt, 2H), 3.61 (t, 2H), 2.53 (s, 3H), 2.35 (s, 9H), 2.05 (t, 2H), 1.99 (td, 2H).

### EXAMPLE 147

### 1-{[3′-(4-Chloro-2,5-dimethyl-benzenesulfonylamino)-3,5-dimethyl-biphenyl-4-carbonyl]-amino}-cyclobutanecarboxylic acid

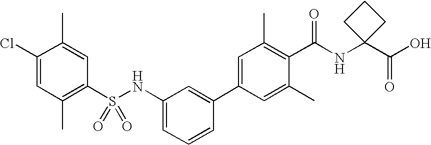

The title compound is obtained by hydrolysis of Example 127.

MS (ESI): 541-543 [M+H]⁺, 1H-NMR (DMSO-d6): δ (ppm) 12.32 (br s, 1H), 10.56 (br s, 1H), 9.00 (s, 1H), 7.99 (s, 1H), 7.51 (s, 1H), 7.32 (t, 1H), 7.26 (d, 1H), 7.24 (s, 1H), 7.11 (s, 2H), 7.06 (d, 1H), 2.60-2.49 (m, 2H), 2.55 (s, 3H), 2.38 (s, 3H), 2.33 (s, 6H), 2.25 (dd, 2H), 1.94 (dd, 2H).

### EXAMPLE 148

### 2-{[3′-(4-Chloro-2,5-dimethyl-benzenesulfonylamino)-3,5-dimethyl-biphenyl-4-carbonyl]-amino}-2-methyl-propionic acid

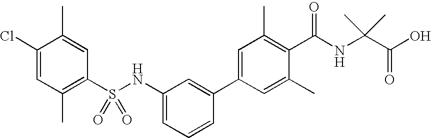

The title compound is obtained by hydrolysis of Example 119

MS (ESI): 529-531 [M+H]⁺, 1H-NMR (DMSO-d6): δ (ppm) 12.24 (br s, 1H), 10.56 (br s, 1H), 8.56 (s, 1H), 7.97 (s, 1H), 7.50 (s, 1H), 7.31 (t, 1H), 7.25 (d, 1H), 7.22 (s, 1H), 7.10 (s, 2H), 7.05 (d, 1H), 2.54 (s, 3H), 2.37 (s, 3H), 2.31 (s, 6H), 1.44 (s, 6H).

### EXAMPLE 149

### 1-{[3′-(4-Chloro-2,5-dimethyl-benzenesulfonylamino)-3,5-dimethyl-biphenyl-4-carbonyl]-amino}-cyclopropanecarboxylic acid

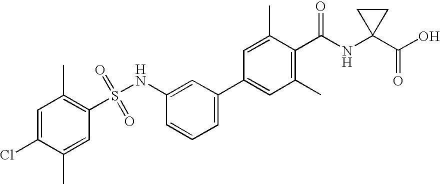

The title compound is obtained by hydrolysis of Example 128.

MS (ESI): 527-529 [M+H]⁺, 1H-NMR (DMSO-d6): δ (ppm) 12.43 (br s, 1H), 10.54 (br s, 1H), 8.81 (s, 1H), 7.96 (s, 1H), 7.49 (s, 1H), 7.30 (t, 1H), 7.26 (d, 1H), 7.21 (s, 1H), 7.08 (s, 2H), 7.04 (d, 1H), 2.53 (s, 3H), 2.35 (s, 3H), 2.29 (s, 6H), 1.40 (dd, 2H), 1.06 (dd, 2H).

### EXAMPLE 150

### 3-{[3′-(4-Chloro-2,5-dimethyl-benzenesulfonylamino)-3,5-dimethyl-biphenyl-4-carbonyl]-amino}-1-methyl-azetidine-3-carboxylic acid

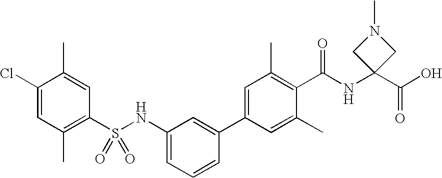

The title compound is obtained by hydrolysis of Example 130.

MS (ESI): 556-558 [M+H]⁺, 1H-NMR (DMSO-d6): δ (ppm) 10.60 (s, 1H), 9.61 (s, 1H), 8.00 (s, 1H), 7.50 (s, 1H), 7.31 (t, 1H), 7.26 (d, 1H), 7.25 (s, 1H), 7.15 (s, 2H), 7.05 (d, 1H), 4.51 (br d, 2H), 4.28 (br d, 2H), 2.90 (s, 3H), 2.52 (s, 3H), 2.35 (s, 9H).

### EXAMPLE 151

### 3′-(4-Chloro-2,5-dimethyl-benzenesulfonylamino)-3,5-dimethyl-biphenyl-4-carboxylic acid ((S)-1-carbamoyl-ethyl)-amide

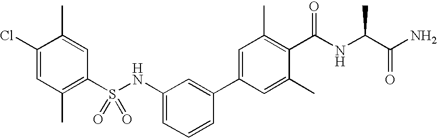

The title compound is prepared according to the procedure described in step 3 of Example 108 using the intermediate acid 28 and (S)-2-Amino-propionamide.

MS (ESI): 512-514 [M−H]⁻, 1H-NMR (DMSO-d6): δ (ppm) 10.55 (brs, 1H), 8.37 (d, 1H), 7.97 (s, 1H), 7.5 (s, 1H), 7.31 (m, 2H), 7.23 (s, 1H), 7.11 (s, 2H), 7.06 (m, 1H), 6.99 (br s, 2H), 4.44 (m, 1H), 2.55 (s, 3H), 2.37 (s, 3H), 2.29 (s, 6H), 1.3 (d, 3H).

### EXAMPLE 152

### 3′-(4-Chloro-2,5-dimethyl-benzenesulfonylamino)-3,5-dimethyl-biphenyl-4-carboxylic acid ((S)-1-methylcarbamoyl-ethyl)-amide

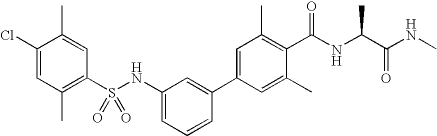

The title compound is prepared according to the procedure described in step 3 of Example 108 using the intermediate acid 28 and (S)-2-Amino-N-methyl-propionamide.

MS (ESI): 526-528 [M−H]⁻, 1H-NMR (DMSO-d6): δ (ppm) 10.55 (br s, 1H), 8.41 (d, 1H), 7.97 (s, 1H), 7.82 (m, 1H), 7.51 (s, 1H), 7.31 (m, 2H), 7.24 (s, 1H), 7.1 (s, 2H), 7.06 (m, 1H), 4.44 (m, 1H), 2.64 (d, 3H), 2.55 (s, 3H), 2.37 (s, 3H), 2.28 (s, 6H), 1.28 (d, 3H).

### EXAMPLE 153

### 3′-(4-Chloro-2,5-dimethyl-benzenesulfonylamino)-3,5-dimethyl-biphenyl-4-carboxylic acid ((S)-1-carbamoyl-2-hydroxy-ethyl)-amide

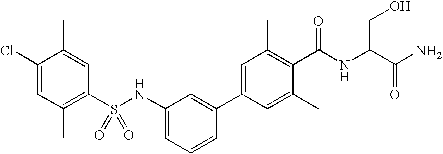

The title compound is prepared according to the procedure described in step 3 of Example 108 using the intermediate acid 28 and (S)-2-Amino-3-hydroxy-propionamide.

MS (ESI): 528-530 [M−H]⁻, 1H-NMR (DMSO-d6): δ (ppm) 10.54 (brs, 1H), 8.15 (d, 1H), 7.96 (s, 1H), 7.49 (s, 1H), 7.28 (m, 4H) 7.1 (s, 3H), 7.05 (m, 1H), 4.85 (t, 1H), 4.45 (m, 1H), 3.66 (m, 2H), 2.53 (s, 3H), 2.35 (s, 3H), 2.29 (s, 6H).

### EXAMPLE 154

### (S)-2-{[3′-(4-Chloro-2,5-dimethyl-benzenesulfonylamino)-3-ethyl-biphenyl-4-carbonyl]-amino}-propionic acid

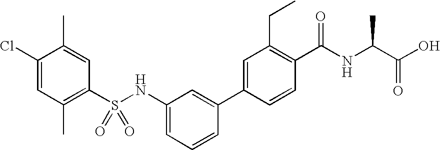

The synthesis of the title compound is accomplished analogously to the synthesis of Example 108 using 4-Bromo-2-ethyl-benzoic acid in step 1 and (S)-2-Amino-propionic acid methyl ester in step 3 followed by LiOH hydrolysis as described in step 2 of Example 48.

MS (ESI): 513-515 [M−H]⁻, 1H-NMR (DMSO-d6): δ (ppm) 12.52 (v br s,1H), 10.59 (br s,1H), 8.6 (d, 1H), 7.97 (s, 1H), 7.51 (s, 1H), 7.29-7.38 (m, 6H), 7.05 (m,1H), 4.39 (m, 1H), 2.79 (q, 2H), 2.55 (s, 3H), 2.36 (s, 3H), 1.36 (d, 3H), 1.18 (t, 3H).

### EXAMPLE 155

### 4-{[3′-(4-Chloro-2,5-dimethyl-benzenesulfonylamino)-3,5-dimethyl-biphenyl-4-carbonyl]-amino}-1-methyl-piperidine-4-carboxylic acid

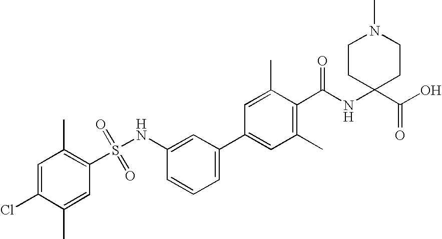

The title compound is obtained by hydrolysis of Example 125 as described in step 2 of Example 48.

MS (ESI): 584-586 [M+H]⁺, 1H-NMR (DMSO-d6): δ (ppm) 10.57 (s, 1H), 8.47 (s, 1H), 7.95 (s, 1H), 7.49 (s, 1H), 7.30 (t,1H), 7.24 (d, 1H), 7.22 (s, 1H), 7.09 (s, 2H), 7.05 (d, 1H), 2.58 (d, 2H), 2.53 (s, 3H), 2.35 (s, 3H), 2.33 (s, 6H), 2.24 (t, 2H), 2.17 (s, 3H), 2.08 (d, 2H), 1.96 (dt, 2H).

### Synthesis of Benzylamine Derivatives

Agents of the invention may conveniently be prepared from the carboxylic acids obtained by the methods described before. Reduction (e.g. with LAH) to the alcohols and oxidation (e.g. with Dess-Martin periodinane) to the aldehydes followed by reductive amination using appropriate amines give the desired products (optionally after a deprotection step) as shown in Reaction Scheme 3 below:

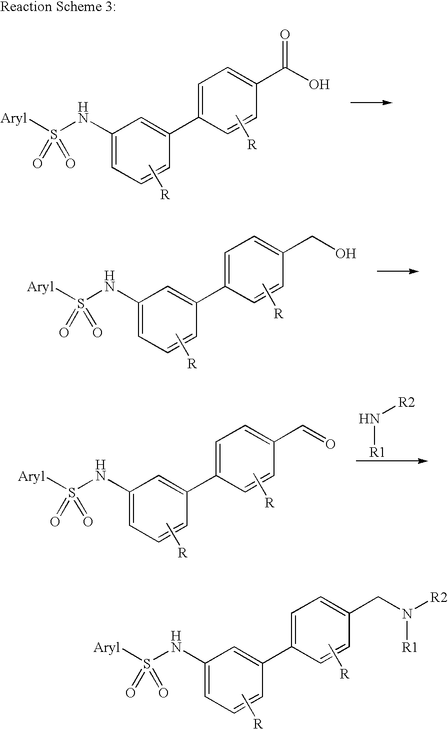

### EXAMPLE 156

### (S)-2-{[3′-(4-Chloro-2,5-dimethyl-benzenesulfonylamino)-biphenyl-4-ylmethyl]-amino}-3-hydroxy-propionic acid

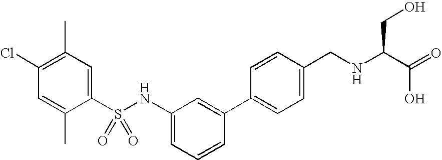

### (1) 4-Chloro-N-(4′-hydroxymethyl-biphenyl-3-yl)-2,5-dimethyl-benzenesulfonamide (26)

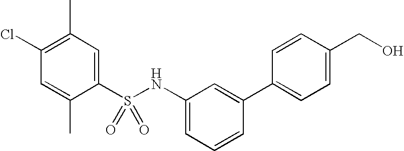

The acid 24 from step 2 of Example 80 (500 mg, 1.19 mmol) is dissolved in THF (12 ml) and lithium aluminium hydride (1M solution in THF, 6.0 ml, 6.00 mmol) is added dropwise. The resulting solution is stirred for 16 hours before dilution with diethyl ether (50 ml). Water (2 ml) is added dropwise to destroy excess reagents, followed by a 8N aqueous sodium hydroxide solution (4 ml). The biphasic medium is filtered and concentrated to an essentially aqueous phase. This is extracted with DCM (50 ml) before the pH is adjusted to 5 with 1N aqueous hydrochloric acid solution. The medium is extracted again with EtOAc (3×50 ml). The combined organic phases are then dried over Na2SO4 and concentrated to give the title product 26 as a yellow oil.

### (2) 4-Chloro-N-(4′-formyl-biphenyl-3-yl)-2,5-dimethyl-benzenesulfonamide (27)

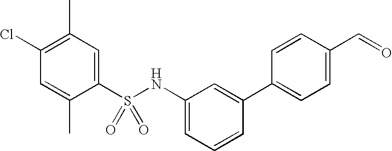

The alcohol 26 (94 mg, 0.21 mmol) is dissolved in DCM (0.640 ml) and treated with Dess-Martin periodinane (101 mg, 0.23 mmol). The resulting solution is stirred at room temperature for 2 hours before dilution with DCM (5 ml) and washing with saturated aqueous sodium bicarbonate solution (2×5 ml). The organic phase is decanted, dried over Na2SO4 and concentrated under vacuum. The crude solid is finally purified by silica gel chromatography ((Hexanes/DCM:5/1)/EtOAc:9/1) to furnish the title product 27 as a yellow solid.

### (3) (S)-2-{[3′-(4-Chloro-2,5-dimethyl-benzenesulfonylamino)-biphenyl-4-ylmethyl]-amino}-3-hydroxy-propionic acid

The aldehyde 27 (48 mg, 0.12 mmol) in solution in THF (800 μl) is treated with (S)-2-Amino-3-tert-butoxy-propionic acid tert-butyl ester (17 mg, 0.08 mmol), acetic acid (80 μl), and polymer-supported sodium cyanoborohydride (Novabiochem, 59 mg, 0.24 mmol). The resulting suspension is shaken for 24 hours at room temperature before the resin is filtered, washed (DCM 3×3 ml) and the organics concentrated under vacuum. The resulting yellow oil is dissolved in TFA (500 μl) and stirred for one hour before concentration and purification by preparative HPLC (Method A). The product-containing fractions are combined, evaporated to dryness, the crude is taken up in tert-butanol and lyophilized to the title compound Example 156, obtained as a white powder. HPLC rt=3.63 min (Method B),

MS (ESI): 489-491 [M+H]⁺.

1H-NMR (DMSO-d6): δ (ppm) 10.62 (br s, 1H), 7.97 (s, 1H), 7.54-7.45 (m, 5H), 7.33 (m, 3H), 7.06 (m, 1H), 4.07 (m, 2H), 3.74 (m, 2H), 3.62 (m, 1H), 2.54 (s, 3H), 2.35 (s, 3H).

### EXAMPLE 157

### (R)-2-{[3′-(4-Chloro-2,5-dimethyl-benzenesulfonylamino)-biphenyl-4-ylmethyl]-amino}-3-hydroxy-propionic acid

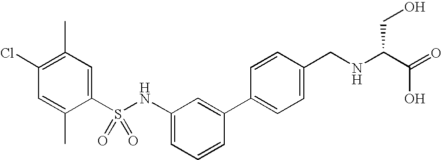

The synthesis of this compound is accomplished analogously to the synthesis of Example 156, using (R)-2-Amino-3-tert-butoxy-propionic acid tert-butyl ester instead of (S)-2-Amino-3-tert-butoxy-propionic acid tert-butyl ester in step 3. HPLC rt=3.66 min (Method B), MS (ESI): 489-491 [M+H]⁺.

1H-NMR (DMSO-d6): δ (ppm) 10.62 (br s, 1H), 7.97 (s, 1H), 7.54-7.45 (m, 5H), 7.33 (m, 3H), 7.06 (m, 1H), 4.07 (m, 2H), 3.74 (m, 2H), 3.62 (m, 1H), 2.54 (s, 3H), 2.35 (s, 3H).

### EXAMPLE 158

### (S)-2-{[3′-(4-Chloro-2,5-dimethyl-benzenesulfonylamino)-3-methyl-biphenyl-4-ylmethyl]-amino}-3-hydroxy-propionic acid

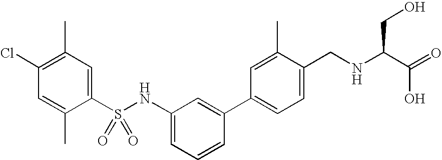

The title compound is obtained from acid 16 from step 3 of Example 45 by reduction and oxidation as described in steps 1 and 2 of Example 156, followed by reductive amination with (S)-2-Amino-3-tert-butoxy-propionic acid tert-butyl ester and TFA-mediated ester hydrolysis.

MS (ESI): 503-505 [M+H]⁺. 1H-NMR (MeOH-d4): δ (ppm) 7.89 (s, 1H), 7.56 (d, 1H), 7.25-7.45 (m, 6H), 7.07 (m, 1H), 4.38 (s, 2H), 4.08 (dd, 1H), 3.94 (dd, 1H), 3.70 (m, 1H), 2.58 (s, 3H), 2.53 (s, 3H), 2.36 (s, 3H).

### EXAMPLE 159

### (R)-2-{[3′-(4-Chloro-2,5-dimethyl-benzenesulfonylamino)-3-methyl-biphenyl-4-ylmethyl]-amino}-3-hydroxy-propionic acid

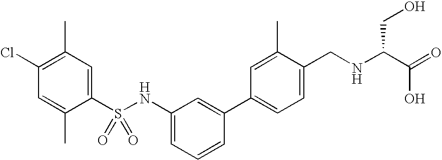

The synthesis of this compound is accomplished analogously to the synthesis of Example 158, using (R)-2-Amino-3-tert-butoxy-propionic acid tert-butyl ester instead of (S)-2-Amino-3-tert-butoxy-propionic acid tert-butyl ester.

MS (ESI): 503-505 [M+H]⁺. 1H-NMR (MeOH-d4): δ (ppm) 7.89 (s, 1H), 7.56 (d, 1H), 7.25-7.45 (m, 6H), 7.07 (m, 1H), 4.38 (s, 2H), 4.08 (dd, 1H), 3.94 (dd, 1H), 3.70 (m, 1H), 2.58 (s, 3H), 2.53 (s, 3H), 2.36 (s, 3H).

### EXAMPLE 160

### (S)-2-{1-[3′-(4-Chloro-2,5-dimethyl-benzenesulfonylamino)-biphenyl-4-yl]-ethylamino}-3-hydroxy-propionic acid

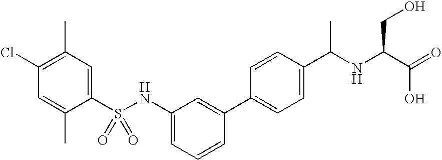

### (1) (S)-2-[1-(4-Bromo-phenyl)-ethylamino]-3-tert-butoxy-propionic acid tert-butyl ester (28)

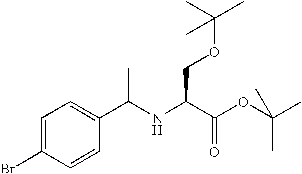

A solution of 4-bromoacetophenone (800 mg, 3.98 mmol), (S)-2-Amino-3-tert-butoxy-propionic acid tert-butyl ester (1166 mg, 5.37 mmol) and 47.5% boron trifluoride diethyletherate solution (120 μl, 0.40 mmol) is refluxed in toluene (13 ml) for 6 hours in a Dien-Stark apparatus. The mixture is then cooled to room temperature, concentrated, taken up in methanol (23 ml) and treated with sodium borohydride (188 mg, 4.77 mmol) for one hour. The medium is then diluted with water (250 ml) and the pH is adjusted to 10 with 8M aqueous sodium hydroxide solution. This is extracted with EtOAc (3×100 ml) and the combined organic phases are dried over Na2SO4 and concentrated to furnish a yellow oil. The product is purified by chromatography on silica gel using a 0% to 25% gradient of EtOAC: 99/NH4OH: 1 in DCM:5/Hexane:1. The product 28 is obtained as a 1/1 mixture of two diastereomers.

### (2) (S)-2-[1-(3′-Amino-biphenyl-4-yl)-ethylamino]-3-tert-butoxy-propionic acid tert-butyl ester (29)

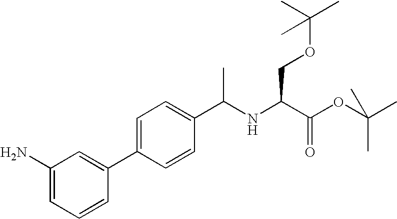

This compound is synthesised in a manner analogous to that used for the synthesis of 14, using 28 instead of 4-bromo-2-methyl-benzoic acid methyl ester.

### (3) (S)-3-tert-Butoxy-2-{1-[3′-(4-chloro-2,5-dimethyl-benzenesulfonylamino)-biphenyl-4-yl]-ethylamino}-propionic acid tert-butyl ester (30)

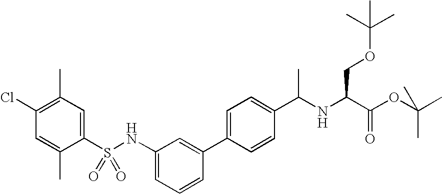

This compound is synthesised in a manner analogous to that used for the synthesis of 15, using 29 instead of 14.

### (4) (S)-2-{1-[3′-(4-Chloro-2,5-dimethyl-benzenesulfonylamino)-biphenyl-4-yl]-ethylamino}-3-hydroxy-propionic acid

The intermediate 30 is treated with TFA for one hour at room temperature. TFA is then evaporated under reduced pressure, the residue is taken up in a mixture of DMA, methanol and water and purification is carried out by preparative reverse-phase HPLC (Method A). The product-containing fractions are then lyophilized to give the title compound Example 160 as a white powder. HPLC rt=3.704 min (Method B), MS (ESI): 503-505 [M+H]⁺.

1H-NMR (DMSO-d6): δ (ppm) 10.64 (s, 1H), 7.97 (br s, 1H), 7.64-7.49 (m, 5H), 7.33 (m, 3H), 7.06 (br d, 1H), 4.53 (m, 1H), 3.95-3.70 (m, 2H), 3.65 (m, 1H), 3.45 (m, 1H), 2.54 (s, 3H), 2.35 (s, 3H), 1.63 (d, 3H).

### EXAMPLE 161

### (S)-2-{1-[3′-(4-Chloro-2,5-dimethyl-benzenesulfonylamino)-biphenyl-4-yl]-pentylamino}-3-hydroxy-propionic acid

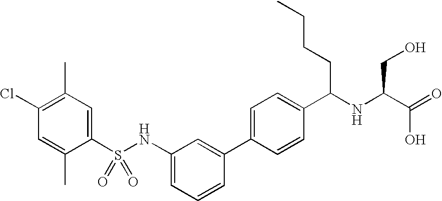

The synthesis of this compound is accomplished analogously to the synthesis of Example 160, using 1-(4-Bromo-phenyl)-pentan-1-one instead of 4-bromoacetophenone in step 1. HPLC rt=4.23 min (Method B), MS (ESI): 545-547 [M+H]⁺.

1H-NMR (DMSO-d6): δ (ppm) 10.61 (s, 1H), 7.97 (br s, 1H), 7.64-7.49 (m, 5H), 7.33 (m, 3H), 7.07 (m, 1H), 4.31 (m, 1H), 3.95-3.10 (m, 3H), 2.55 (s, 3H), 2.35 (s, 3H), 2.21 (m, 1H), 1.97 (m, 1H), 1.25 (m, 2H), 1.11 (m, 1H), 0.90 (m, 1H), 0.78 (m, 3H).

### EXAMPLE 162

### (S)-2-{[3′-(4-Chloro-2,5-dimethyl-benzenesulfonylamino)-3,5-dimethyl-biphenyl-4-ylmethyl]-amino}-propionic acid

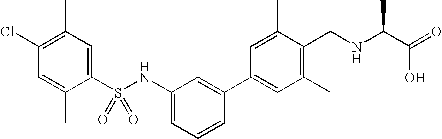

### (1) 4-Chloro-N-(4′-hydroxymethyl-3′,5′-dimethyl-biphenyl-3-yl)-2,5-dimethyl-benzenesulfonamide (int #32)

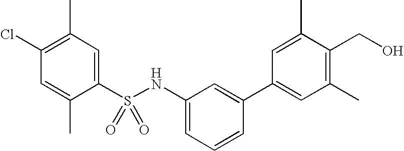

To a suspension of the acid 28 from step 2 of Example 108 (1.2 g, 2.7 mmol) in DCM (30 ml) and a catalytic amount of DMF (3 drops) is added thionylchloride (392 μl, 5.41 mmol) and the mixture is heated to reflux for 60 minutes upon which all solid is dissolving. The formation of the acid chloride intermediate is checked by quenching an aliquot with methanol and analyse the sample as the methyl ester. The solvents are then evaporated and dried under high vacuum for about 15 minutes. The resulting foam is dissolved in THF (20 ml) and cooled in an ice-bath. A solution of sodium borohydride (511 mg, 13.51 mmol) in DMF (3 ml) is slowly added and stirring is continued for 15 minutes. The reaction mixture is then hydrolysed with 2N—HCl and diluted with EtOAc (50 ml). The organic layer is separated, washed twice with water brine, dried over sodium sulphate, filtered and evaporated. The crude title product int #32 is used without further purification.

MS (ESI): 429-431 [M+H]⁺.

### (2) 4-Chloro-N-(4′-formyl-3′,5′-dimethyl-biphenyl-3-yl)-2,5-dimethyl-benzenesulfonamide (int #33)

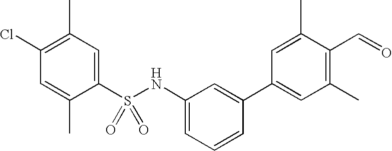

The alcohol int #32 (500 mg, 1.16 mmol) is dissolved in DCM (12 ml) and treated with Dess-Martin periodinane (592 mg, 1.4 mmol). The resulting suspension is stirred at room temperature for 16 hours. Another portion of Dess-Martin periodinane (246 mg, 0.7 mmol) is added and the mixture is heated to reflux for 3 hours. The newly formed precipitate is filtered off and the solvent is evaporated. The brown residue is then dissolved in ethyl acetate and washed with 10% sodium carbonate solution, 2N—HCl and brine. The organic phase is separated, dried over sodium sulphate and evaporated. The crude is finally purified by silica gel chromatography using cyclohexane/ethyl acetate from 2% to 10% to furnish the title product int #33 as a white solid.

MS (ESI): 426-428 [M−H]⁻.

### (3) (S)-2-{[3′-(4-Chloro-2,5-dimethyl-benzenesulfonylamino)-3,5-dimethyl-biphenyl-4-ylmethyl]-amino}-propionic acid methyl ester (int #34)

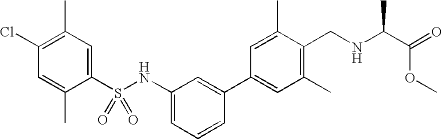

A solution of the aldehyde int #33 (43 mg, 0.1 mmol) in DCM (1 ml) is treated with (S)-2-Amino-propionic acid methyl ester (14 mg, 0.1 mmol) and sodium triacetoxy borohydride (53 mg, 0.25 mmol). The mixture is stirred for 24 hours at room temperature. Another equivalent of (S)-2-amino-propionic acid methyl ester and sodium triacetoxy borohydride is added and stirring is continued for 2 hours. The reaction mixture is then diluted with ethyl acetate (10 ml) washed with 2N—HCl, 10% sodium carbonate solution and brine. The organic layer is dried over sodium sulphate, filtered and evaporated. The crude is finally purified by silica gel chromatography using cyclohexane/ethyl acetate from 2% to 15% to furnish the title compound int #34 as a white powder.

MS (ESI): 513-515 [M−H]⁻.

### (4) (S)-2-{[3′-(4-Chloro-2,5-dimethyl-benzenesulfonylamino)-3,5-dimethyl-biphenyl-4-ylmethyl]-amino}-propionic acid

The ester int #34 (20 mg, 0.039 mmol) is dissolved in THF and treated with 1N—LiOH solution (0.16 ml, 0.16 mmol). After stirring for 2 hours most of the THF is evaporated and the residue is diluted with water (5 ml) and washed with ether (5 ml). The aqueous layer is separated the pH is adjusted to 3-5 with 2N—HCl and extracted twice with ethyl acetate (10 ml). The organic layers are dried over sodium sulphate, filtered and evaporated to furnish the title product Example 162 as white powder.

MS (ESI): 499-501 [M−H]⁻, 1H-NMR (DMSO-d6): δ (ppm) 10.58 (br s, 1H), 7.98 (s, 1H), 7.51 (s, 1H), 7.33 (m, 2H), 7.26 (s, 1H), 7.18 (s, 2H), 7.06 (m, 1H), 4.16 (m, 1H), 4.05 (m, 1H), 3.85 (m, 1H), 2.55 (s, 3H), 2.48 (s, 6H), 2.36 (s, 3H), 1.45 (d, 3H).

### EXAMPLE 163

### (R)-2-{[3′-(4-Chloro-2,5-dimethyl-benzenesulfonylamino)-3,5-dimethyl-biphenyl-4-ylmethyl]-amino}-propionic acid

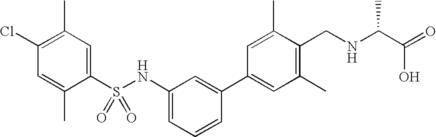

The synthesis of the title compound is performed analogously to Example 162 but using (R)-2-Amino-propionic acid methyl ester in step 3.

MS (ESI): 499-501 [M−H]⁻, 1H-NMR (DMSO-d6): δ (ppm) 10.58 (br s, 1H), 7.98 (s, 1H), 7.51 (s, 1H), 7.33 (m, 2H), 7.26 (s, 1H), 7.18 (s, 2H), 7.06 (m, 1H), 4.16 (m, 1H), 4.05 (m, 1H), 3.85 (m, 1H), 2.55 (s, 3H), 2.48 (s, 6H), 2.36 (s, 3H), 1.45 (d, 3H).

### EXAMPLE 164

### (S)-2-{[3′-(4-Chloro-2,5-dimethyl-benzenesulfonylamino)-3,5-dimethyl-biphenyl-4-ylmethyl]-methyl-amino}-propionic acid

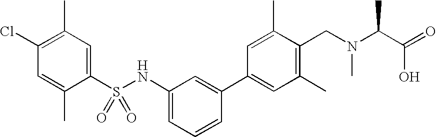

The synthesis of the title compound is performed analogously to Example 162 but using (S)-2-Methylamino-propionic acid methyl ester in step 3.

MS (ESI): 513-515 [M−H]⁻.

### EXAMPLE 165

### (R)-2-{[3′-(4-Chloro-2,5-dimethyl-benzenesulfonylamino)-3,5-dimethyl-biphenyl-4-ylmethyl]-amino}-propionic acid

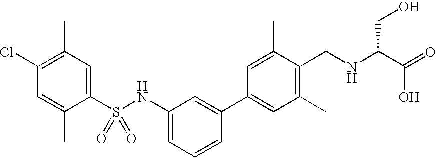

The synthesis of the title compound is performed analogously to Example 162 but using (R)-2-Amino-3-hydroxy-propionic acid methyl ester in step 3.

MS (ESI): 515-517 [M−H]⁻, 1H-NMR (DMSO-d6): δ (ppm) 10.53 (brs, 1H), 7.96 (s, 1H), 7.49 (s, 1H), 7.3 (m, 2H), 7.23 (s, 1H), 7.11 (s, 2H), 7.04 (m, 1H), 5.0 (v brs, 1H), 3.9 (dd, 2H), 3.75 (m, 1H), 3.65 (m, 1H), 3.36 (m, 1H, overlapping with water signal), 2.53 (s, 3H), 2.43 (s, 6H), 2.35 (s, 3H).

### EXAMPLE 166

### 1-[3′-(4-Chloro-2,5-dimethyl-benzenesulfonylamino)-biphenyl-4-ylmethyl]-azetidine-3-carboxylic acid

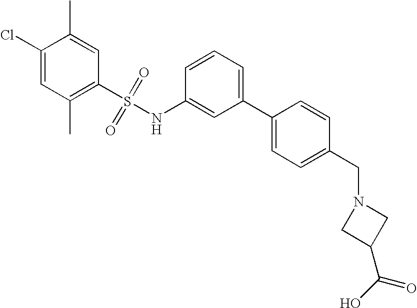

The synthesis of this compound is accomplished analogously to the synthesis of Example 162, using the aldehyde 27 from Example 156 and azetidine-3-carboxylic acid methyl ester

MS (ESI): 485-487 [M+H]⁺, 1H-NMR (DMSO-d6): δ (ppm) 10.54 (br s, 1H), 7.93 (s, 1H), 7.46 (s, 1H), 7.39 (d, 2H), 7.31 (d, 2H), 7.28 (s, 1H), 7.25 (t, 1H), 7.23 (d, 1H), 7.01 (d, 1H), 3.56-3.20 (m, 7H), 2.53 (s, 3H), 2.34 (s, 3H).

### EXAMPLE 167

### 1-[3′-(4-Chloro-2,5-dimethyl-benzenesulfonylamino)-3-methyl-biphenyl-4-ylmethyl]-azetidine-3-carboxylic acid

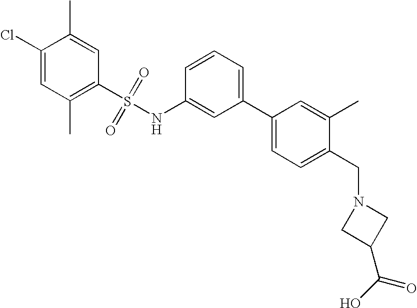

The title compound is obtained from acid 16 from step 3 of Example 45 by reduction and oxidation as described in steps 1 and 2 of Example 156, followed by reductive amination with azetidine-3-carboxylic acid methyl ester and ester hydrolysis as described in steps 3 and 4 of Example 162.

MS (ESI): 499-501 [M+H]⁺, 1H-NMR (DMSO-d6): δ (ppm) 10.49 (br s, 1H), 7.93 (s, 1H), 7.47 (s, 1H), 7.30-7.20 (m, 6H), 7.00 (d, 1H), 3.54-3.22 (m, 7H), 2.54 (s, 3H), 2.35 (s, 3H), 2.31 (s, 3H).

### EXAMPLE 168

### 4-[3′-(4-Chloro-2,5-dimethyl-benzenesulfonylamino)-biphenyl-4-ylmethyl]-morpholine-3-carboxylic acid

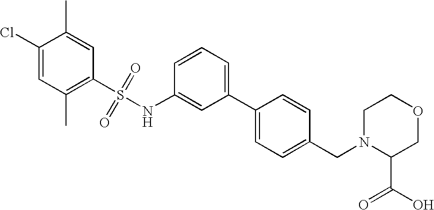

The synthesis of this compound is accomplished analogously to the synthesis of Example 162, using the aldehyde 27 from Example 156 and morpholine-3-carboxylic acid methyl ester.

MS (ESI): 515-517 [M+H]⁺, 1H-NMR (DMSO-d6): δ (ppm) 10.60 (brs, 1H), 7.94 (s, 1H), 7.50 (s, 1H), 7.48 (d, 4H), 7.32 (s, 1H), 7.30 (t, 1H), 7.29 (d, 1H), 7.05 (d, 1H), 4.18-3.00 (m, 9H), 2.54 (s, 3H), 2.35 (s, 3H).

### EXAMPLE 169

### 4-[3′-(4-Chloro-2,5-dimethyl-benzenesulfonylamino)-biphenyl-4-ylmethyl]-morpholine-2-carboxylic acid

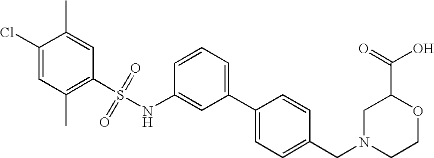

The synthesis of this compound is accomplished analogously to the synthesis of Example 162, using the aldehyde 27 from Example 156 and morpholine-2-carboxylic acid methyl ester.

MS (ESI): 515-517 [M+H]⁺, 1H-NMR (DMSO-d6): δ (ppm) 12.72 (brs, 1H), 10.53 (brs, 1H), 7.93 (s, 1H), 7.46 (s, 1H), 7.42 (d, 2H), 7.38 (d, 2H), 7.28 (s, 1H), 7.27 (t, 1H), 7.23 (d, 1H), 7.02 (d, 1H), 4.08 (dd, 1H), 3.89 (dt, 1H), 3.59-3.50 (m, 4H), 2.75 (dd, 1H), 2.54 (s, 3H), 2.34 (s, 3H), 2.32-3.18 (m, 2H).

### EXAMPLE 170

### (2S,3S)-1-[3′-(4-Chloro-2,5-dimethyl-benzenesulfonylamino)-biphenyl-4-ylmethyl]-3-hydroxy-pyrrolidine-2-carboxylic acid

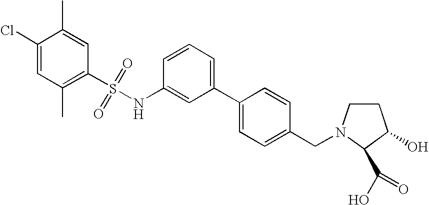

The synthesis of this compound is accomplished analogously to the synthesis of Example 162, using the aldehyde 27 from Example 156 and (2S,3S)-3-hydroxy-pyrrolidine-2-carboxylic acid methyl ester.

MS (ESI): 515-517 [M+H]⁺, 1H-NMR (DMSO-d6): δ (ppm) 10.53 (brs, 1H), 7.94 (s, 1H), 7.49-7.40 (m, 5H), 7.30-7.25 (m, 3H), 7.02 (d, 1H), 5.30 (br s, 1H), 4.29 (d, 1H), 4.04 (d, 1H), 3.95 (d, 1H), 3.26 (d, 1H), 3.20 (m, 1H), 2.90 (m, 1H), 2.54 (s, 3H), 2.34 (s, 3H), 1.86 (m, 1H), 1.70 (m, 1H).

### EXAMPLE 171

### (2S,4 R)-1-[3′-(4-Chloro-2,5-dimethyl-benzenesulfonylamino)-biphenyl-4-ylmethyl]-4-hydroxy-pyrrolidine-2-carboxylic acid

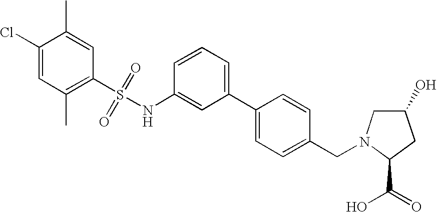

The synthesis of this compound is accomplished analogously to the synthesis of Example 162, using the aldehyde 27 from Example 156 and (2S,4R)-4-hydroxy-pyrrolidine-2-carboxylic acid methyl ester.

MS (ESI): 515-517 [M+H]⁺, 1H-NMR (DMSO-d6): δ (ppm) 10.54 (brs, 1H), 7.93 (s, 1H), 7.46 (s, 1H), 7.42 (d, 2H), 7.41 (d, 2H), 7.29 (s, 1H), 7.27 (t, 1H), 7.25 (d, 1H), 7.03 (d, 1H), 4.93 (br s, 1H), 4.20 (m, 1H), 4.03 (d, 1H), 3.66 (d, 1H), 3.47 (t, 1H), 3.15 (dd, 1H), 2.34 (s, 3H), 2.36 (m, 1H), 2.34 (s, 3H), 2.05-1.90 (m, 2H).

The compounds of formula I in free form or in pharmaceutically acceptable salt form, exhibit valuable pharmacological properties, e.g. as S1P1 receptor antagonists, e.g. as indicated in vitro and in vivo tests and are therefore indicated for therapy.

A. In Vitro

The compounds of formula I have typically binding affinity to human S1P receptors as determined in following assays:

Human S1P Receptor Calcium FLIPR Antagonist Assays

HeLa Gα16 S1P1:

The assay measures intracellular changes of Ca²⁺ mediated by the synthetic probing agonist 3-{[2-(2-Trifluoromethyl-biphenyl-4-yl)-benzo[b]thiophen-5-ylmethyl]-amino}-propionic acid (GNF-AC-1) in the HeLa-S1P1/Gα16 cell clone 1: HeLa (human cervix carcinoma, ATCC CCL2) cells stably expressing N-terminally myc-tagged human S1P1 receptors (GenBank™ accession No. NM_{—}001400; UNIPROT P21453) and promiscuous Gα16 protein (GenBank™ accession number M63904, Swissprot P30679) are cultured at 37° C., 5% CO2, and 95% relative humidity. The cells are plated in 384 well black plates (10,000 cells per well). After 24 hours the cells are loaded with Fluo4-AM (1.6 μM in HBSS and 2.5 mM probenicid) for 1 hour at 37° C. After washing, the cells are transferred to the FLIPR. The test compounds are added at different concentrations (≦30 μM) in HBSS in the presence of 0.1% BSA and changes in fluorescence are recorded (indication of agonism). The probing agonist is added 15 minutes afterwards to the wells at a concentration giving 80% of the maximal activity (EC₈₀). After each addition time points are collected as follows: 20 time points (2 seconds) before the addition of the agonist (Fmin) and 60 time points (1 or 2 seconds) after the addition of the probing agonist. This allows the determination of the maximal fluorescence (Fmax). The ratio (Fmax-Fmin)/Fmin is plotted against the log of the concentration of the test compounds and the IC₅₀ (relative antagonism) is determined with the help of XLfit-4 software. Compounds with an inhibition <20% are usually considered “inactive”. A dose response curve of the probing agonist is determined on each plate in parallel. The compounds of the invention are typically active in this assay at a concentration ranging typically from <1 nM to 30 μM, usually from less than 1 nanomolar to 1 micromolar.

The compounds described above have the following IC₅₀ values in the above described Human S1P Receptor Calcium FLI PR Antagonist Assay:

| Example No. | IC₅₀ (nanomolar) |
|---|---|
| 23 | 3.2 |
| 25 | 8.8 |
| 28 | 2.8 |
| 36 | 9.4 |
| 45 | 1.1 |
| 46 | 0.8 |
| 47 | 1.5 |
| 48 | 9 |
| 51 | 3 |
| 55 | 5 |
| 56 | 5 |
| 57 | 0.8 |
| 59 | 0.7 |
| 60 | 1.5 |
| 61 | 0.4 |
| 62 | 0.5 |
| 63 | 1.3 |
| 64 | 1.3 |
| 66 | 5.5 |
| 70 | 1 |
| 71 | 1 |
| 79 | 5 |
| 85 | 1.7 |
| 86 | 1.5 |
| 131 | 0.6 |
| 132 | 1.1 |
| 133 | 3.5 |
| 134 | 7 |
| 135 | 1.7 |
| 136 | 1.7 |
| 137 | 1 |
| 138 | 1.1 |
| 139 | 1.1 |
| 142 | 5.5 |
| 143 | 5 |
| 144 | 4 |
| 145 | 2 |
| 146 | 2 |
| 147 | 3.5 |
| 148 | 5 |
| 149 | 1.7 |
| 150 | 1.6 |
| 151 | 8 |
| 153 | 3 |
| 154 | 1.7 |
| 155 | 2 |
| 157 | 4.7 |
| 165 | 10 |

CHO S1P1 Assay:

The assay measures intracellular changes of Ca²⁺ mediated by the endogenous agonist S1P in the CHO-K1 cells (ATCC CCL 61) stably expressing human S1P1 (GenBank™ accession No. NM_{—}001400; UNIPROT P21453). Cells are cultured at 37° C., 5% CO2, and 95% relative humidity. The cells are plated in 384 well black plates (10,000 cells per well). After 24 hours the cells are loaded with Fluo4-AM (1.6 μM in HBSS and 2.5 mM probenicid) for 1 hour at 37° C. After washing, the cells are transferred to the FLIPR. The test compounds are added at different concentrations (≦30 μM) in HBSS in the presence of 0.1% BSA. After 10 min the cells are treated with 10 μM ATP. S1P is added 30 minutes afterwards to the wells at a concentration giving 80% of the maximal activity (EC₈₀). After each addition time points are collected as follows: 20 time points (2 seconds) before the addition of the agonist (Fmin) and 60 time points (1 or 2 seconds) after the addition of the agonist. This allows the determination of the maximal fluorescence (Fmax). The ratio (Fmax-Fmin)/Fmin is plotted against the log of the concentration of the test compounds and the IC₅₀ (relative antagonism) is determined with the help of XLfit-4 software.Compounds with an inhibition <20% are typically considered “inactive”. A dose response curve of S1P is determined on each plate in parallel. The compounds of the invention are usually active in this assay at a concentration ranging typically from <1 nM to 30 μM, usually from less than 1 nanomolar to 1 micromolar.

CHO hS1P4 and CHO hS1P5 Assays:

These assays are performed exactly as described for the CHO S1P1 cells. Human S1P5 cDNA (GenBank™ accession number AY262689, UNIPROT: Q9H228) and human S1P4 cDNA (GenBank™ accession Number AJ000479, UNIPROT:095977) are used to generate stable CHO-K1 cells (ATCC CCL 61)cells lines. The compounds of the invention are typically active in this assay usually at a concentration >1 μM, preferably more than 10 micromolar, typically more than 30 micromolar.

CHO hS1P3 and CHO hS1P2 Assays:

The assay measures intracellular changes of Ca²⁺ mediated by the endogenous agonist S1P in the CHO-K1 cells (ATCC CCL 61) cells stably expressing human S1P3 (GenBank™ accession Numbers: X83864 and UNIPROT:Q99500) and human S1P2 (GenBank™ accession Numbers: AF034780, UNIPROT:095136). Cells are cultured at 37° C., 5% CO2, and 95% relative humidity. The cells are plated in 384 well black plates (10,000 cells per well). After 24 hours the cells are loaded with Fluo4-AM (1.6 μM in HBSS and 2.5 mM probenicid) for 1 hour at 37° C. After washing, the cells are transferred to the FLIPR. The test compounds are added at different concentrations (≦30 μM) in HBSS in the presence of 0.1% BSA and changes in fluorescence are recorded (indication of agonism). S1P is added 20 minutes afterwards to the wells at a concentration giving 80% of the maximal activity (EC₈₀). After each addition time points are collected as follows: 20 time points (2 seconds) before the addition of the agonist (Fmin) and 60 time points (1 or 2 seconds) after the addition of the agonist. This allows the determination of the maximal fluorescence (Fmax). The ratio (Fmax-Fmin)/Fmin is plotted against the log of the concentration of the test compounds and the IC₅₀ (relative antagonism) is determined with the help of XLfit-4 software. Compounds with an inhibition <20% are typically considered “inactive”. A dose response curve for S1P is determined on each plate in parallel. The compounds of the invention are usually active in this assay typically at a concentration >1 μM, preferably more than 10 micromolar, typically more than 30 micromolar.

Human S1P1 GTPγ³⁵S Binding Assay:

This human S1P1 dependent GTPγ-³⁵S binding assay measures functional human S1P1 antagonists, e.g. compounds that interfere with S1P induced GTPγ-³⁵S binding. This assay is based on scintillation proximity and measures S1P induced GTPγ-³⁵S to CHO membranes stably expressing S1P1 after the addition of the probing agonist S1P and different concentration of antagonistic compounds. Membrane proteins obtained from CHO cells expressing human S1P1 are absorbed to lectin-bead impregnated with scintillation fluid (SPA bead) and distributed in a 96 well plate. Different concentrations of testing compounds are added to the beads/membrane mixture and gently mixed for 15 min before addition of 0.5 nM to 5 nM S1P (˜EC50 and ˜EC90, respect.). After a further incubation for 15 min GTPγ-³⁵S is added to start the assay. The reaction is stopped by centrifugation after 2 h and plates are measured with a TopCount NXT instrument. The compounds of the invention are typically active in this assay typically at a concentration ranging typically from <1 nM to 30 μM, usually from less than 1 nanomolar to 1 micromolar.

B. In Vivo

The compounds of formula I usually induce the depletion of blood lymphocyte as determined in the following assay:

Measurement of Circulating Lymphocytes:

The test compounds (or salts thereof) are dissolved in a vehicle such as water, saline, PEG (polyethylene glycol) 200, or PBS (phosphate buffered saline). Rats (Lewis strain, male, 6-12 weeks old) are administered up to 100 mg/kg of the test compounds in 2 ml/kg vehicle via subcutaneous application. The vehicle or a reference salt (reference salt is N-methyl-D-glucamin acetate) dissolved in saline and FTY720 (0.3 mg/kg) are included as negative and positive controls, respectively.

Blood is collected from the sublingual vein 0, 2, 8 and 24 hours after the test compound administration under short isoflurane anesthesia. Whole blood samples are subjected to hematology analysis. Peripheral lymphocyte counts are determined using an automated analyzer. The Haemathology System uses a combination of light scatter, cytochemical staining and nuclear density on two independent channels to measure the total and differential white cell counts. Two to four rats are used to assess the lymphocyte depletion activity of each compound screened. The result is an ED₅₀, which is defined as the effective dose that induces 50% reduction of blood lymphocyte counts. Compounds of formula I tested according to the above assay have typically an ED₅₀ of less than 50 mg/kg.

The compounds of formula I are, therefore, useful in the treatment and/or prevention of diseases or disorders mediated by lymphocytes interactions, e.g. in transplantation, such as acute or chronic rejection of cell, tissue or organ allo- or xenografts or delayed graft function, graft versus host disease, autoimmune diseases, e.g. rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves ophthalmopathy, alopecia areata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, infectious diseases, e.g. toxic shock (e.g. superantigen induced), septic shock, adult respiratory distress syndrome or viral infections, e.g. AIDS, viral hepatitis, chronic bacterial infection, or senile dementia. Examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus. Furthermore, the compounds of formula I are useful in the treatment and/or prevention of diseases or disorders associated with deregulated angiogenesis for example diseases caused by ocular neovascularisation, especially retinopathies (diabetic retinopathy, age-related macular degeneration); psoriasis; haemangioblastomas, such as “strawberry-marks” (=haemangioma); various inflammatory diseases, such as arthritis, especially rheumatoid arthritis, arterial atherosclerosis and atherosclerosis occurring after transplants, endometriosis or chronic asthma; and, especially, tumor diseases (solid tumors, but also leukemias and other liquid tumors).

The present invention preferably provides:
1.1 A method for preventing or treating acute or chronic transplant rejection in a subject in need of such treatment, which method comprises administering to said subject an effective amount of a compound of formula I or a pharmaceutically acceptable salt thereof;
1.2 A method for preventing or treating autoimmune diseases, such as rheumatoid arthritis, systemic lupus erythematosus, psoriasis, or multiple sclerosis in a subject in need of such treatment, which method comprises administering to said subject an effective amount of a compound of formula I or a pharmaceutically acceptable salt thereof;
1.3 A method for preventing or treating multiple sclerosis in a subject in need of such treatment, which method comprises administering to said subject an effective amount of a compound of formula I or a pharmaceutically acceptable salt thereof;
2. A compound of formula 1, in free form or in a pharmaceutically acceptable salt form for use as a pharmaceutical, e.g. in any of the methods as indicated under 1.1, 1.2 or 1.3 above.
3. A pharmaceutical composition, e.g. for use in any of the methods as in 1.1, 1.2 or 1.3 above comprising a compound of formula I in free form or pharmaceutically acceptable salt form in association with a pharmaceutically acceptable diluent or carrier therefor.
4. A compound of formula I or a pharmaceutically acceptable salt thereof for use in the preparation of a pharmaceutical composition for use in any of the method as in 1.1, 1.2 or 1.3 above.
5. A method as defined above comprising co-administration, e.g. concomitantly or in sequence, of a therapeutically effective non-toxic amount of a compound of formula I and at least a second drug substance, e.g. an immunosuppressant, immunomodulatory, anti-inflammatory or chemotherapeutic drug, e.g. as indicated below.
6. A pharmaceutical combination, e.g. a kit, comprising a) a first agent which is a compound of formula I as disclosed herein, in free form or in pharmaceutically acceptable salt form, and b) at least one co-agent, e.g. an immunosuppressant, immunomodulatory, anti-inflammatory, chemotherapeutic or anti-infectious agent. The kit may comprise instructions for its administration.

For the above uses the required dosage will of course vary depending on the mode of administration, the particular condition to be treated and the effect desired.

In general, satisfactory results are indicated to be obtained systemically at daily dosages of from about 0.03 to 5.0 mg/kg per body weight. An indicated daily dosage in the larger mammal, e.g. humans, is in the range from about 0.5 mg to about 500 mg, conveniently administered, for example, in divided doses up to four times a day or in retard form. Suitable unit dosage forms for oral administration comprise from ca. 0.1 to 50 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form e.g. as indicated above. Such salts may be prepared in conventional manner and exhibit the same order of activity as the free compounds. A preferredroute of administration for these comounds is parenterally, using a salt, for example a N-methyl-D-glucamine salt or D-glucamine salt.

The compounds of formula I may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g a malignant cell anti-proliferative agent. For example, the compounds of formula I may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578, AP23573, AP23464, AP23675, AP23841, TAFA-93, biolimus-7 or biolimus-9; an ascomycin having immunosuppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a co-agent, are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

1. A compound of formula I or a pharmaceutically acceptable salt thereof, 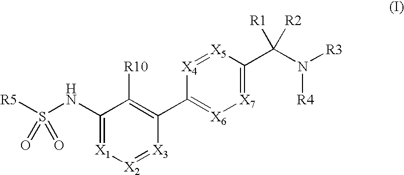
wherein
X₁, X₂, X₃, X₄, X₅, and X₇ are each independently N or CR6, each R6 is independently selected from the group consisting of H, halo, cyano, OH and; (C₁-C₆ alkyl, C₁-C₆ alkoxy, aryl C₁-C₆ alkoxy, heteroaryl C₁-C₆ alkoxy and C₁-C₆ alkylamine, which are optionally substituted with C₁-C₆ alkoxy, OH, halo, cyano, sulfonyl, C₁-C₆ alkyl, amino, mercapto or COOH;
R1 and R2 are each independently H or C₁-C₆ alkyl, or taken together are ═O;
R3 is C₁-C₆ alkyl optionally substituted in any position with one or more substituents R3′, each R3′ being independently selected from the group consisting of COOR11, CON(R12)₂, hydroxyl, amino, aryl, heteroaryl, cycloalkyl, heterocycloalkyl, aryl C₁-C₆ alkyl, heteroaryl C₁-C₆ alkyl, C₁-C₆ alkyl, C₁-C₆ alkoxy, halo, cyano, mercapto, and sulfonyl and;
R3′ are optionally substituted once or more with COOR11, CON(R12)₂, hydroxyl, amino, aryl, heteroaryl, cycloalkyl, heterocycloalkyl, aryl C₁-C₆ alkyl, heteroaryl C₁-C₆ alkyl, C₁-C₆ alkyl, C₁-C₆ alkoxy, halo, cyano, mercapto and sulfonyl; or
two R3′ may form together with the carbon atoms to which they are attached a 3-8 membered saturated or unsaturated carbocyclic ring optionally containing up to 2 ring members selected from CO, CHCOOR11, NR12, O, S, SO or SO₂;
wherein R11 is independently H, C₁-C₆ alkyl or benzyl; and R12 is independently H, OH, C₁-C₆ alkyl, benzyl or acyl;
R4 is H, acyl or C₁-C₆ alkyl;
or R3 and R4 are linked together to form a 4, 5, 6 or 7 membered carbocyclic or heterocyclic ring which is optionally substituted by one or more groups R340 ;
R5 is aryl or heteroaryl, in which the aryl or heteroaryl is optionally substituted with one or more groups independently selected from the group consisting of halo, C₁-C₆ alkyl, NO₂, C₁-C₆ alkoxy, cyano, amino, sulfonyl, aryl, heteroaryl and mercapto,
wherein the substituents on R5 are themselves optionally substituted by with halo, NO₂, C₁-C₆ alkoxy, cyano, amino, sulfonyl, aryl or heteroaryl;
R10 is H or (C₁-C₆ alkyl, C₁-C₆ alkoxy, aryl C₁-C₆ alkoxy, heteroaryl C₁-C₆ alkoxy, C₁-C₆ alkylamine; which are oDtionallv substituted with C₁-C₆ alkoxy, OH, halo, cyano, sulfonyl, C₁-C₆ alkyl, amino, mercapto or COOH.

2. The compound according claim 1 having the structure of formula II or a pharmaceutically acceptable salt thereof, 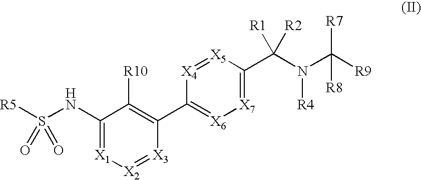
wherein R7 is H or C₁-C₆ alkyl, aryl, aryl C₁-C₈ alkyl heteroaryl, heteroaryl C₁-C₆ alkyl, which are optionally substituted with OH, C₁-C₆ alkoxy or N(R12)₂;
R8 is H or C₁-C₆ alkyl;
or R7 and R8 form together with the carbon atoms to which they are attached a 3-8 membered saturated or unsaturated ring optionally containing up to 2 ring members selected from CO, CHCOOH, CHCOOR11, NR12, O, S, SO or SO₂; and
R9 is COOR11, CON(R12)₂ or tetrazole;
wherein R11 is independently H, C₁-C₆ alkyl or benzyl; and R12 is independently H, OH, C₁-C₆ alkyl, benzyl or acyl.

3. The compound according to claim 1 having the structure of formula IlIl or a pharmaceutically acceptable salt thereof,

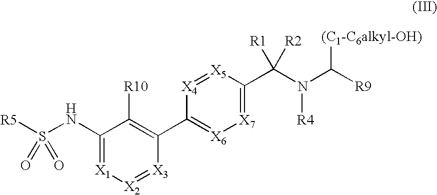

4. The compound according to claim 1 having the structure of formula (IIIa) or a pharmaceutically acceptable salt thereof;

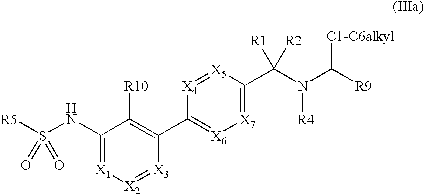

5. The compound according to claim 1 having the structure of formula (IIIb) or a pharmaceutically acceptable salt thereof; 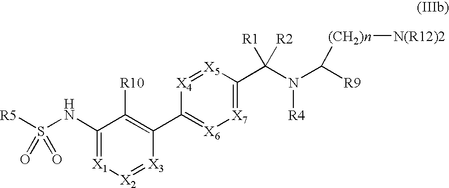
wherein n is 1 or 2.

6. The compound according to claim 1 having the structure of formula (IIIc) or a pharmaceutically acceptable salt thereof; 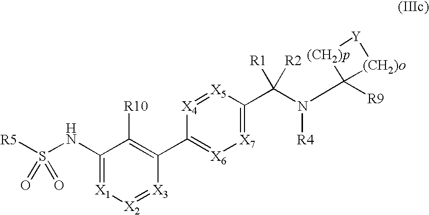
wherein o and p are integers and are independently 0, 1, 2, 3, 4 or 5 with the proviso that the sum of o+p is from 1 to 5; and Y is CH₂, CO, CHCOOH, CHCOOR11, NR12, O, S, SO or SO₂.

7. (canceled)

8. A combination comprising the compound according to claim 1 and an active agent selected from: an immunosuppressive or immunomodulating agent, anti-inflammatory agent, chemotherapeutic agent, calcineurin inhibitor, mTOR inhibitor, corticosteroid; PKC inhibitor, JAK3 kinase inhibitor, immunosuppressive monoclonal antibody, adhesion molecule inhibitor, or an anti-infectious agent for simultaneous, separate or sequential use.

9. A compound according to claim 1 or a pharmaceutically-acceptable and -cleavable ester thereof for use as a pharmaceutical.

10. (canceled)

11. A method of treatment of a disease or disorder mediated by lymphocytes interactions comprising: administering to a patient in need thereof, an effective amount of the compound according to claim 1, or a salt thereof.

12. The method of claim 11, wherein said disease or said disorder is transplantation, acute or chronic rejection of cell, tissue or organ allo- or xenografts or delayed graft function, graft versus host disease, autoimmune diseases, rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves ophthalmopathy, alopecia areata and others, allergic diseases, allergic asthma, atopic dermatitis, allergic rhinitis/conjunctivitis, allergic contact dermatitis, inflammatory diseases optionally with underlying aberrant reactions, inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, myocardial infarction, stroke, gut ischemia, renal failure or hemorrhage shock, traumatic shock, cancer, e.g. breast cancer, T cell lymphomas or T cell leukemias, infectious diseases, toxic shock, septic shock, adult respiratory distress syndrome or viral infections, AIDS, viral hepatitis, chronic bacterial infection, or senile dementia.

13. A pharmaceutical composition, comprising: the compound according to claim 1 or a pharmaceutically-acceptable salt thereof and a pharmaceutically acceptable excipient, diluent or carrier.

14. The method of claim 11 wherein the said disease or said disorder is acute or chronic transplant rejection.

15. The method of claim 11 wherein the said disease or said disorder is an autoimmune disease selected from the group consisting of rheumatoid arthritis, systemic lupus erythematosus, psoriasis and multiple sclerosis.

16. The method of claim 15 wherein the said disease or said disorder is multiple sclerosis.
